(19) United States
(12) Patent Application Publication
(10) Pub. No.: US 2008/0299137 A1
(43) Pub. Date: Dec. 4, 2008

(54) Fusion Proteins That Bind Effector Lymphocytes And Target Cells

(75) Inventors: Ivan Svendsen, Vaerlose (DK); Vibeke Westphal Stennicke, Kokkedal (DK); Peter Andreas Nicolai Reumert Wagtmann, Rungsted Kyst (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/091,820

(22) PCT Filed: Oct. 27, 2006
(86) PCT No.: PCT/EP2006/067892

§ 371 Date: Jul. 18, 2008

Related U.S. Application Data
(60) Provisional application No. 60/732,176, filed on Nov. 1, 2005.

(30) Foreign Application Priority Data
Oct. 28, 2005 (EP) 05110146.7

(51) Int. Cl. A61K 39/44; C07K 16/46; A61K 31/711; C12P 21/00; A61P 35/00

(52) U.S. Cl. 424/178.1; 530/391.7; 514/44; 435/69.7

(57) ABSTRACT

Novel fusion proteins that comprise a first portion that corresponds to an antibody-like protein that is specific for an activating receptor on an effector lymphocyte or a variant thereof and a second portion that corresponds to a portion of a cell membrane protein and that binds to a cell-associated target are provided, as are methods of producing such fusion proteins, uses and methods involving such fusion proteins, and compounds and compositions related to such fusion proteins.

### FIELD OF THE INVENTION

This invention relates to new pharmaceutical compositions, proteins, and methods of producing and using such compositions and proteins, as well as additional related compositions and methods.

### BACKGROUND OF THE INVENTION

Effector lymphocytes include T cells, such as cytotoxic T lymphocytes (CTLs), natural killer (NK cells), and natural killer T (NKT) cells. These cells play critical roles in the immune system's capacity to protect the body against disease-causing agents such as cancer cells and viruses.

Bispecific antibodies (antibodies comprising antibody sequences specific for two distinct targets) are well known in the art. Bispecific antibodies having one part directed to activating receptors expressed on effector lymphocytes and another part specific for antigens on tumor cells can prove efficacious in the treatment of various disorders. For example, an anti-CD16/anti-CD30 bispecific antibody has been demonstrated to be very effective in treatment of patients with refractory Hodgkin's lymphoma. Exemplary other bispecific antibodies are, e.g., those that bind both malignant B-cell lymphomas and T cells (U.S. Pat. No. 6,129,914). Unfortunately, the generation of bispecific antibodies composed entirely of human antibody sequences can be laborious.

A number of fusion proteins comprising Fc antibody domains bound to non-antibody portions (often referred to as “immunoadhesins”) also have been described. U.S. Pat. No. 5,225,538, for example, described fusion proteins of LHR and Ig Fc regions; and Kurschner et al. (J. Immunol., 1992; 149(12):4096-4100) described fusion proteins of the IFN-gamma receptor with constant Ig domains. Recently, Regunathan et al. described fusion proteins comprising the ectodomain of the natural killer (NK) cell activating receptor NGK2D and the Fc portion of human immunoglobulin G (IgG) (Blood, 105(1):233-240 (Jan. 1, 2005)). US Patent Publication Nos. 20040072256 and 20020142445 similarly disclose fusion proteins of NK cell activating receptors NKp30, NKp44, and NKp46 and Ig Fc portions. In a somewhat related context, US Patent Publication No. 20040138417 describes heteromultimer adhesins, wherein sequences that correspond to different portions of a heteromultimeric receptor are associated by a multimerization domain derived from an antibody constant region, and US Patent Publication No. 20030195338 somewhat similarly describes Fc-linked receptor sequence proteins. Still other types of Fc-based fusion proteins are described in Japanese Patent Application JP2005206478 and at world-wide web address scancell.co.u k/pages/products/immunobody_vaccines.htm#.

Multispecific molecules produced from the fusion of antigen-binding portions of anti-bodies to other non-antibody proteins also are known. Dreier et al. (Bioconjug. Chem. 9(4): 482-489 (1998)), for example, describe the construction of recombinant mouse cytokines IL2 and GM-CSF as fusion proteins with the carboxyl terminus of a chimeric rat/mouse antibody, ch17217, directed against the transferrin receptor. U.S. Pat. No. 6,046,310 describes fusion proteins comprising a FAS ligand portion and a portion derived from variable regions of an antibody. US Patent Publication No. 20030103984 purportedly describes antibody-peptide fusion proteins, including antigen-binding portions of an antibody and a portion of a peptide involved with “immunostimulatory, membrane transport, and homophilic activities” (though only fusion proteins comprising a small amino acid sequence from complement peptide C3d appear to be described in any detail). European Patent Application 1 413 316 and US Patent Publication No. 20040038339 suggest fusion proteins comprising antibodies against a tumor cell antigen and MHC I-related protein portions derived from an NKG2D-ligand such as MIC-A, MIC-B, or ULBP; von Strandmann et al., Blood (2005-05-2177; pre-published online on Oct. 6, 2005) describes fusion proteins comprising an anti-CD138 antibody linked to ULBP; and International Patent Application WO 2004056873 suggests fusion proteins comprising one portion binding to an NK cell antigen and one portion binding to CD2, CD4, CD44, CD69 or the T-cell receptor.

Non-antibody multispecific fusion proteins comprising portions of an effector lymphocyte activating receptor have also been described. For example, US Patent Publication No. 20040115198 describes, e.g., NKG2D-DAP10 fusion proteins and fusions of the trans-membrane or cytoplasmic domain of NKG2D with distinct extracellular ligand binding domains.

Other types of antibody fusion proteins or multispecific molecules are exemplified in U.S. Pat. Nos. 6,407,221 and 5,359,046 (membrane-bound antibody fusion proteins); US Patent Application 2002187151 (e.g., multivalent NKG2D-ligands);, and 6881828 (e.g., fusion protein comprising an antigenic peptide linked to a B2M peptide).

Despite the foregoing, there remains a need for alternative and improved multispecific molecules capable of facilitating activation of effector lymphocytes against selected populations of target cells. As described above, literature has suggested, e.g., fusion proteins that bind the NKG2D-receptor on effector lymphocytes via an NKG2D-ligand such as ULBP, and tumor cells via e.g. a CD138-specific antibody. The flexibility of such constructs can be limited, however, since the NKG2D-receptor is often downregulated in cancer (Groh et al., Nature 2002 419:734-8). Further, the antibody portion is typically specific for a single antigen whose expression might not be tumor-specific, and antigen expression on tumors may be reduced or lost in, e.g., so-called “escape variants” of the tumor. To address these and other problem in the art, the invention provides new and useful molecules, methods of producing such molecules, and methods of using such molecules. These and other inventive features, aspects, and further benefits thereof will be apparent to the ordinarily skilled artisan upon thorough review of the disclosure provided herein.

### SUMMARY OF THE INVENTION

In one exemplary aspect of the invention, novel fusion proteins that comprise a portion that corresponds to at least a portion of an antibody-like protein (“antibody portion”) and is capable of specifically binding an effector lymphocyte activating receptor and a second portion (“target-binding portion”) that corresponds to at least a functional and/or ligand-binding segment of the extracellular domain of a cell-membrane-associated protein or a functional variant thereof are provided. Exemplary cell-membrane-associated proteins are those that are Type II receptors (i.e. receptors having their amino terminus presented on the cytoplasmic side of the cell and the carboxy terminus on the exterior), and/or those whose ligands are expressed on tumor cells, virally infected cells, or other target cells.

The invention further relates to new methods of producing such fusion proteins, using such fusion proteins in various applications (e.g., the treatment of diseases such as viral infections, cancers, etc.), and various additional compounds, compositions, and methods related to such fusion proteins (e.g., nucleic acids coding for the production of inventive fusion proteins, related vectors, host cells, etc.).

These and additional exemplary inventive aspects and features of the invention are further illuminated by the following list of illustrative aspects of the invention and extensively described in other parts of this document.

### DESCRIPTION OF THE DRAWINGS

FIG. 1 is a schematic representation of an exemplary fusion protein comprising a portion of the human NKG2D receptor (target-binding portion) and an anti-human CD3 binding antibody portion (effector lymphocyte-specific antibody portion), the construction of which is described in the Experimental Data and Methods section.

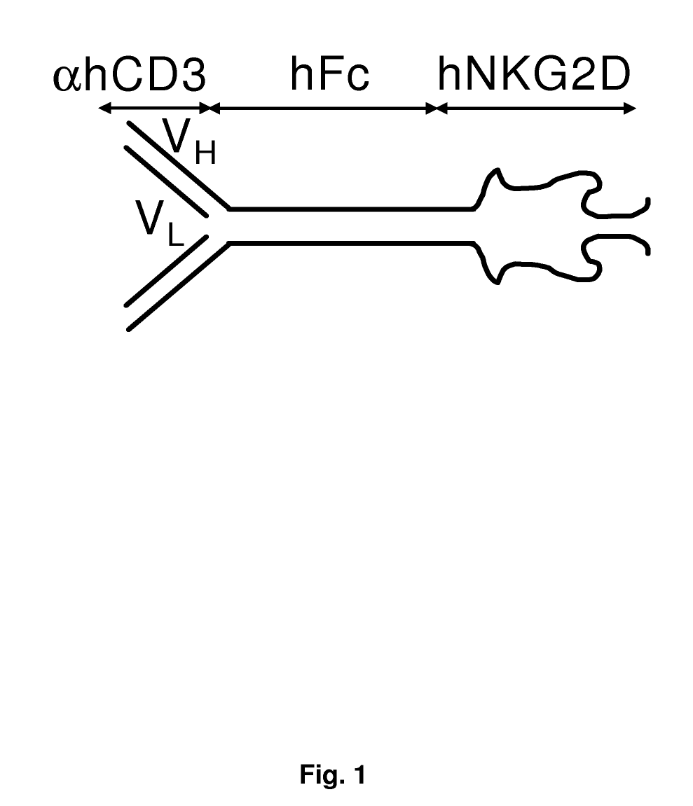

FIG. 2 is a schematic presentation of the expression plasmid aCD3Vk in pTT5-LC encoding the Vk chain of an anti-mouseCD3 antibody.

FIG. 3 is a schematic presentation of the expression plasmid amCD3-mFcm-NKG2D HC in pTT5-HC encoding the exemplary fusion protein referenced above with respect to FIG. 1.

FIG. 4 shows the growth and killing of KLN205 (target cells) incubated with murine T cells. After 18 hours of growth, freshly purified murine T cells (effector cells) were added at different effector-to-target ratios.

FIG. 5 shows normalized growth and killing of KLN205 (target cells) by murine T cells after addition of an fusion protein comprising an anti-murine CD3 antibody portion and a murine NKG2D target-binding portion. After 18 hours of growth, freshly purified murine T cells (effector cells) were added (E:T 10:1), and 4 hours after addition of effector cells the fusion protein or control was added.

FIG. 6 shows normalized growth and killing of HEPA1-6 (target cells) by murine T cells after addition of fusion protein. After 18 hours of growth, freshly purified murine T cells (effector cells) were added (E:T 1:1), and 4 hours after addition of effector cells the fusion protein or control was added.

### DESCRIPTION OF THE INVENTION

The invention described herein has many facets, including the provision of new protein and protein-related molecules (e.g., various protein derivatives), pharmaceutical compositions and other compositions, new methods and uses of such compositions, related compositions to the new proteins (e.g., nucleic acid molecules encoding the same, cells comprising such nucleic acids, etc.), and the like. Although various exemplary aspects of the invention are described separately herein, the reader will appreciate that the various teachings provided herein may be combined with one another as suitable unless otherwise stated or clearly indicated.

The invention is based, in part, on fusion proteins that are designed to specifically “label” a target cell such as, e.g., a tumor cell or virally infected cell, for destruction by the immune system. This is accomplished by fusing a portion of an antibody (or antibody-like protein) that can specifically bind to an effector lymphocyte to a ligand-binding portion of a receptor whose ligand is expressed on target cells.

Thus, in one exemplary aspect, the invention provides novel proteins that comprise an antibody-like “first portion” that specifically binds to an effector lymphocyte activating receptor and a different “second portion” that corresponds to at least a functional portion of the extracellular domain of a cell membrane protein or a functional variant thereof. The second portion, herein referred to as “target-binding portion”, is capable of binding at least one cell-associated target (“secondary target”) that is different from the effector lymphocyte activating receptor. The second portion may also be referred to as the “membrane protein extracellular domain portion”. When both the first and the second portions of the fusion proteins are bound to the activating receptor on effector cells and to the antigen on target cells, the former will cross-link the activating receptor, triggering the effector cells to kill the specific antigen presenting cells.

The “first portion” of the inventive fusion proteins is an activating receptor-binding antibody like protein portion. While not limited to any particular type of antibody or antibody-like protein; for simplicity, this portion is herein referred to as the “antibody portion.” An antibody-portion does not specifically bind a target-binding protein contained in the fusion protein.

In general, the antibody and target-binding portions may be contained within a fusion protein of the invention in any suitable manner and have any suitable type of conformation, structure, etc. “Suitability” with respect to the positioning and physical properties of the antibody and target-binding portions generally is determined by the ability of the fusion protein in question to (a) at least bind an effector lymphocyte activating receptor and (b) bind a receptor or ligand for the target-binding portion (desirably with sufficient affinity under physiological conditions so as to promote a desired physiological effect). An advantage of at least some fusion proteins of the invention is the ability to bind a target presented on cells associated with a particular disease, condition, or disorder that is regulated by effector lymphocytes under normal conditions (e.g., a cancer, a viral infection, etc.) with a target-binding portion while also binding to and desirably activate an activating receptor on an effector lymphocyte (such as an NK cell), so as to promote the immune system of an individual to act upon such disease-associated cells, thereby inducing or promoting a therapeutic effect. Suitability also may take into considerations factors such as immunogenicity and other undesirable properties (e.g., toxicity), stability, shelf-life, etc.

In one aspect, antibody and target-binding portions comprise individual amino acid sequences, associated amino acid sequences (e.g., by multimerization, cysteine-cysteine bonding, or other mechanism), or (respectively) a combination thereof, wherein the sequences comprised in the portions are directly associated with one another. In another aspect, the antibody and target-binding portions are separated by a linker residue, sequence, or suitable non-amino acid moiety linker. In one aspect, the invention provides fusion proteins comprising a single antibody portion bound to a single target-binding portion, across one or more associated protein chains, wherein the antibody portion and target-binding portion are oriented N-terminal to C-terminal, respectively. In a particular exemplary facet, the invention provides fusion proteins wherein a single antibody portion (whether on one or more protein chains) is bound directly to the N-terminus of an target-binding portion, wherein the antibody portion and/or target-binding portion may be optionally located at the termini of the fusion protein.

Antibody portion(s) and target-binding portions can each comprise any suitable number of target-binding (i.e., effector-lymphocyte activating receptor-binding and secondary target-binding) amino acid sequences distributed on any number of protein chains. Each portion or both portions (or multiple instances of one or more of such portions where applicable) can, for example, comprise one, two, or more amino acid sequences distributed on one or two associated protein chains. In one exemplary aspect, the invention provides fusion proteins that include antibody portions comprising more than two effector lymphocyte activating receptor-binding sequences (e.g., 3, 4, 6, 8, 10, 12 sequences, etc.) distributed in one or more associated protein chains (e.g., 2, 4, 6, 10, or more chains).

In one aspect, fusion proteins of the invention can be characterized in comprising an antibody portion that does not bind to one or more of the effector-lympohocyte receptors bound by any fusion proteins described in the references cited in the Background of the Invention and/or an target-binding protein that does not bind to one or more of the targets bound by the fusion proteins described in the references cited in the Background of the Invention. In other and separate aspect, some fusion proteins of the invention may be characterized by having an antibody portion not binding NKG2D, a target-binding portion not binding NKG2D, an antibody portion not binding CD138, a target-binding portion not binding CD138, or any combination thereof.

For sake of convenience, these two types of fusion protein “portions” are described separately in detail in the following sections.

### Antibody Portion

An antibody portion of a fusion protein of the invention refers to a part of such a fusion protein that comprises one or more amino acid sequences, which sequence(s) is/are capable of binding to an effector lymphocyte activating receptor, and which sequence(s) correspond(s) to at least a portion of an antibody or antibody-like protein.

Terms such as “antibody-like protein” herein refer to proteins that are able to specifically bind to an effector lymphocyte activating receptor, but that are not a naturally occurring endogenous ligand for the receptor or a protein that is highly similar to a naturally occurring endogenous ligand for the receptor (e.g., an antibody-like typically exhibits about 75% or less, about 70% or less, about 65% or less, about 60% or less, about 55% or less, about 50% or less, about 45% or less, or even about 40% or less amino acid sequence identity to a naturally occurring ligand for the effector lymphocyte activating receptor). Typical antibody like proteins include antibodies and other protein molecules that act similarly to antibodies in terms of specific binding of proteins, such as affibodies, anticalins, or trinectins. Particular examples of such antibody-like proteins are described elsewhere herein.

Typically, an antibody portion comprises one or more amino acid sequences that corresponds to a functional portion of an antibody. An antibody is an immunoglobulin molecule that is produced by a cell in response to an antigen or an essentially equivalent molecule (e.g., a synthetically produced immunoglobulin molecule that essentially corresponds to an immunoglobulin molecule produced by such a cell). Immunoglobulins are a class of structurally related proteins comprising heavy chains (e.g., α, Δ, ε, γ, and μ chains) and light chains (e.g., κ and λ chains). In humans, immunoglobulins may be divided into five major classes (IgA, IgD, IgE, IgG, and IgM) according to which heavy chains are contained in the Ig molecule. The structure of immunoglobulins is well characterized. See, e.g., FUNDAMENTAL IMMUNOLOGY (Paul, W., ed., 2nd ed. Raven Press, N.Y. (1989)). The numbering of amino acid residues in the variable region of a naturally occurring antibody (which comprises the complementarity determining regions (CDRs) interspersed with the conserved framework regions (FR)) can be conveniently performed using the method described in Kabat et al., SEQUENCES OF PROTEINS OF IMMUNOLOGICAL INTEREST, 5th Ed. Public Health Service, National Institutes of Health, Bethesda, Md. (1991) (phrases such as “variable domain residue numbering as in Kabat,” “according to Kabat,” and the like herein refer to this numbering system for heavy chain variable domains or light chain variable domains). Using this numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of identity of the sequence of the antibody with a “standard” Kabat numbered sequence.

Unless otherwise stated or indicated, the term “antibody” herein includes polyclonal antibodies and monoclonal antibodies (mAbs). The term “monoclonal antibody” refers to a composition comprising a homogeneous antibody population having a uniform structure and specificity. Polyclonal antibodies have mixed specificity. Polyclonal antibodies typically are derived from the serum of an animal that has been immunogenically challenged. Monoclonal antibodies can be produced by various known means, such as through hybridoma technology, phage display technology, or synthesis methods, examples of which are described elsewhere herein and/or are known in the art.

An antibody in the context of this invention can possess any isotype and an antibody of interest of a particular isotype can be “isotype switched” with respect to an original anti-body from which it is derived using conventional techniques. Such techniques include the use of direct recombinant techniques (see e.g., U.S. Pat. No. 4,816,397), cell-cell fusion techniques (see e.g., U.S. Pat. No. 5,916,771), and other suitable techniques known in the art. Typically, an antibody-like protein corresponds to at least a portion of a human IgG isotype antibody.

An antibody-like protein may advantageously correspond to a human antibody produced in a transgenic animal designed to produce human antibodies, an example of such animal system being the XenoMouse™ (Abgenix—Fremont, Calif., USA) (see, e.g., Green et al. Nature Genetics 7:13-21 (1994); Mendez et al. Nature Genetics 15:146-156 (1997); Green and Jakobovits J. Exp. Med. 188:483-495 (1998); European Patent No., EP 0 463 151 B1; International Patent Application Nos. WO 94/02602, WO 96/34096; WO 98/24893, WO 99/45031, WO 99/53049, and WO 00/037504; and U.S. Pat. Nos. 5,916,771, 5,939,598, 5,985,615, 5,998,209, 5,994,619, 6,075,181, 6,091,001, 6,114,598 and 6,130,364)). An antibody-like protein also may correspond to a portion of a humanized antibody or chimeric antibody.

The antigen-binding function of antibodies can be performed by any number of suitable “fragments” thereof. Accordingly, a antibody portion can comprise, consist, or consist essentially of a functional “fragment” of an antibody. An antibody “fragment” can be characterized as a protein that comprises a functional portion of a “full length” antibody molecule. The term “fragment” is not intended to define how such a molecule is made.

Antibody “fragments” can be obtained by actual fragmentation of an antibody molecule, by recombinant production of a portion of an antibody molecule, or by another suitable technique. Antibodies can be fragmented using conventional techniques, for example, and the fragments screened for utility in the same manner as described elsewhere herein with respect to “whole” or “full length” antibodies with respect to the ability to appropriate bind a desired target. For example, F(ab′)2 fragments can be generated by treating antibody with pepsin. The resulting F(ab′)2 fragment can be treated to reduce disulfide bridges to produce Fab′ fragments. Fab fragments can be obtained by treating an IgG antibody with papain; F(ab′) fragments can be obtained with pepsin digestion of IgG antibody. A F(ab′) fragment also can be produced by binding Fab′ via a thioether bond or a disulfide bond. A Fab′ fragment is an antibody fragment obtained by cutting a disulfide bond of the hinge region of ment obtained by cutting a disulfide bond of the hinge region of the F(ab′)2. A Fab′ fragment can be obtained by treating a F(ab′)2 fragment with a reducing agent, such as dithiothreitol.

Antibody fragments can also be generated by expression of nucleic acids encoding such peptides in recombinant cells (see, e.g., Evans et al., J. Immunol. Meth. 184: 123-38 (1995)). For example, a chimeric gene encoding a portion of a F(ab′)2 fragment can include DNA sequences encoding the CH1 domain and hinge region of the H chain, followed by a translational stop codon to yield such a truncated antibody fragment molecule.

Examples of known antibody fragments include (i) a Fab fragment, a monovalent fragment consisting essentially of the VL, VH, CL and CH I domains; (ii) F(ab)₂ and F(ab′)2 fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., (1989) Nature 341:544-546), which consists essentially of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain antibodies or single chain Fv (scFv); see e.g., Bird et al. (1988) Science 242:423-426: and Huston et al. (1988) Proc. Natl. Acad. Sci. USA 85:5879-5883). Such single chain antibodies also are encompassed within terms such as antibody fragment and antibody-like peptide/molecule, unless otherwise noted or clearly indicated by context. Other forms of single chain antibodies, such as diabodies also are intended to be generally encompassed by these terms. Diabodies are bivalent, bispecific antibodies in which VH and VL domains are expressed on a single polypeptide chain, but using a linker that typically is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123; and Cao et al. (1998), Bioconjugate Chem. 9, 635-644). Although having similar target molecule binding properties as full-length antibodies, fusion proteins comprising antibody fragment first portions collectively and each independently can be considered unique features of the invention, typically exhibiting different biological and/or physiochemical properties and utilities than fusion proteins comprising “complete” or near complete antibody first portions.

Each effector lymphocyte activating receptor-binding amino acid sequence comprised by an antibody portion can be of any suitable length and composition. Typically, a single receptor-binding portion is about 50-500 amino acids in length, such as about 100-450 amino acids, in length. A receptor-binding antibody portion also can comprise, however, a number of subsequences (e.g., 2, 3, 4, 5, 6, or more subsequences) that collectively contribute to binding the first cellular target. Such first target-binding subsequences may be distributed on one or more (e.g., 2) chains in the first portion of a antibody portion. In one exemplary aspect, the invention provides a fusion protein comprising two associated peptide chains that each comprise a first effector lymphocyte activating receptor-binding subsequence of about 75-150 amino acids in length, wherein the chain-separated receptor-binding portions interact to specifically bind one or more targets (e.g., a single target or multiple targets in the case where the first portion exhibits cross-reactivity for a desired subset of targets). In another illustrative aspect, the invention provides fusion proteins that comprise a single protein chain comprising two first effector lymphocyte activating receptor-binding sub-sequences separated by a spacer/linker, wherein the separated first target-binding portions interact to specifically bind one or more targets.

In one embodiment, the antibody portion does not itself activate the effector lymphocyte activating receptor upon binding. Instead, only when both the first and the second portions of the fusion proteins are bound to the activating receptor on effector cells and to the antigen on target cells, the former will cross-link the activating receptor, triggering the effector cells to kill the specific antigen presenting cells. In an alternative embodiment, the antibody activates the receptor upon binding. Standard functional assays to evaluate the target cell-killing capability by lymphocytes in the presence and absence of antibody or fusion protein can be set up to assess and/or screen for the ability of the antibody portion to activate the receptor to which it binds (see, e.g., Examples 2 and 4).

The antibody portion can correspond to or be derived from (i.e., be a variant and/or derivative of) any suitable type of effector lymphocyte activating receptor-binding antibody-like protein. In one aspect, the invention provides fusion proteins comprising an antibody portion that corresponds to or is derived from an antibody against an activating receptor expressed on an NK cell, a T cell, and/or a NKT cell.

In one aspect, the invention provides fusion proteins comprising a antibody portion comprising amino acid sequences that correspond to at least a portion of an antibody against a peptide presented (i.e., displayed) on a T cell of a mammal (e.g., a human) or a functional fragment thereof. In a particular aspect, the invention provides fusion proteins comprising an antibody portion that corresponds to at least a portion of an antibody specific for a portion of a T cell receptor (TCR) or a functional variant thereof. In one advantageous exemplary aspect, the invention provides fusion proteins comprising an antibody portion that comprises or corresponds to at least a portion of an antibody that is specific for an invariable portion of a TCR, such as CD3 or an invariable gamma-delta TCR chain or that comprises or corresponds to a functional variant of such an antibody portion.

The sequence and composition of various TCRs and TCR subunits have been described or are known (see, e.g., GenBank Accession Nos. AAW31109, AAW31108, AAW31107, AAW31106, AAW31105, AAW31104, and AAW31103; and U.S. Pat. No. 5,169,938) and various methods for producing antibodies against TCRs have been previously developed (including, recently, the production of antibodies against soluble TCRs and, even more recently, against so-called monoclonal TCRs). Such proteins can readily be used to produce antibodies, from which TCR-specific first portions can be derived for inclusion into a fusion protein according to the invention. Exemplary anti-TCR antibody production methods, antibodies, and related principles are described in, e.g., Necker et al., Eur J. Immunol. 1991 December; 21 (12):3035-40; Brodnicki et al., Mol. Immunol. 1996 February; 33(3):253-63 and Mol Immunol 1996 May-June; 33(7-8):735 (erratum); Tsang et al., Vet Immunol Immunopathol. 2005 Jan. 10; 103(1-2):113-127; Pavlistova et al., Immunol Lett. 2003 Aug. 5; 88(2):105-8; Kubo et al., J. Immunol. 1989 Apr. 15; 142(8):2736-42; and U.S. Pat. Nos. 5,616,472; 5,766,947; 5,980,892; and 6,392,020. Antibodies against TCRs also are currently commercially available. Examples of commercially available anti-TCR Abs include Serotec catalog numbers (MCA987; MCA987T; MCA990; MCA990T; MCA990F; MCA990FT (Serotec, Varilhes, France).

As also indicated elsewhere herein, one advantageous aspect of the invention is embodied in fusion proteins comprising an antibody portion that is specific for CD3. Anti-CD3 antibodies, anti-CD3 antibody fragments, derivatives of such proteins, and principles related to the production and use of such antibodies are known (see, e.g., Dunstone et al., Acta Crystallogr D Biol Crystallogr. 2004 August; 60(Pt 8):1425-8; Le Gall et al., J Immunol Methods. 2004 Feb. 1; 285(1):111-27; Renders et al., Clin Exp Immunol. 2003 September; 133(3):307-9; Norman et al., Transplantation. 2000 Dec. 27; 70(12):1707-12; Cole et al., J. Immunol. 1997 Oct. 1; 159(7):3613-21; Arakawa et al., J Biochem (Tokyo). 1996 September; 120(3):657-62; Adair et al., Hum Antibodies Hybridomas. 1994; 5(1-2):41-7; US Patent Publication Nos. 20040202657, 20040175786, 20040058445, and 20030216551, International Patent Application WO 91/09968, and U.S. Pat. Nos. 6,890,753; 6,750,325; 6,706,265; 6,406,696; 6,143,297; 6,113,901; 5,968,509; 5,929,212; 5,834,597; 5,658,741; 5,585,097; and 5,527,713. An example of a commercially available anti-CD3 antibody is the murine OKT3 antibody. Light chain and heavy chain variable sequences from OKT3 are Ala Ser Gly Val Pro Ala His Phe Arg Gly Ser Gly Ser Gly Thr Ser Tyr Ser Leu Thr Ile Ser Gly Met Glu Ala Glu Asp Ala Ala Thr Tyr Tyr Cys Gln Gln Trp Ser Ser Asn Pro Phe Thr Phe Gly Ser Gly Thr Lys Ile Ile Asn Arg Ala (SEQ ID NO:1) and Gln Val Gln Val Gln Gln Ser Gly Ala Glu Leu Ala Arg Pro Gly Ala Ser Val Lys Met Ser Cys Lys Ala Ser Gly Tyr Thr Phe Thr Arg Tyr Thr Met Leu Gly Met His Trp Val Lys Gln Arg Pro Gly Gln Gly Leu Glu Trp Ile Gly Tyr Ile Asn Pro Ser Arg (SEQ ID NO:2), respectively (see also GenBank Accession No. BAA11539). Such sequences, or highly similar sequences that retain specificity for a target CD3, can form, in whole or in part, an antibody portion in a fusion protein according to one aspect of the invention.

In another exemplary aspect, a fusion protein comprising an antibody portion that specifically binds a CD3 is provided, which antibody portion comprises an anti-CD3 antibody heavy chain region that comprises, consists, or consists essentially of the sequence Met Ala Gln Val Gln Leu Gln Gln Ser Gly Pro Glu Leu Val Lys Pro Gly Thr Ser Val Lys Met Ser Cys Lys Ala Ser Gly Tyr Lys Phe Ile Ser Tyr Val Ile His Trp Val Lys Gln Lys Pro Gly Gln Gly Leu Glu Trp Ile Gly Tyr Ile Asn Pro Tyr Asn Ala Val Thr Lys Tyr Asn Glu Lys Phe Lys Gly Lys Ala Thr Leu Thr Ser Asp Lys Ser Ser Ser Thr Ala Ser Met Glu Leu Ile Ser Leu Thr Ser Glu d Ser Thr Val Tyr Tyr Cys Thr Arg Ser Asp Tyr Tyr Asp Tyr Asp Gly Phe Ala Tyr Trp Gly Gln Gly Thr Leu Ile Thr Val Ser Ala Ala Lys Thr Thr Pro Pro Ser Val Tyr Pro Leu Ser Arg Gly Ser Arg—SEQ ID NO:3) or a functional portion thereof and an anti-CD3 light chain region comprising, consisting, or consisting essentially of the sequence Gln Ile Val Leu Thr Gln Ser Pro Ala Ile Met Ser Ala Ser Pro Gly Glu Lys Val Thr Met Thr Cys Ser Ala Ser Ser Ser Val Ser Tyr Ile His Trp Tyr Gln Gln Lys Ser Gly Thr Ser Pro Lys Arg Trp Ile Tyr Asp Ile Ser Lys Leu Ala Ser Gly Val Pro Val Arg Phe Ser Gly Ser Gly Ser Gly Thr Ser Tyr Ser Leu Thr Ile Ser Ser Val Glu Ala Glu Asp Ala Ala Thr Tyr Tyr Cys Gln Gln Trp Ser Thr Asn Pro Pro Thr Phe Gly Ala Gly Thr Lys Leu Val Leu Lys Arg Ala Asp Ala Ala Pro Thr Val Ser—SEQ ID NO:4) or a functional portion thereof.

In yet another aspect, the invention provides fusion proteins comprising a CD3-specific antibody portion that comprises the “heavy chain” CDRs comprising, consisting, or consisting essentially of the sequences (a) Ser-Phe-Pro-Met-Ala (SEQ ID NO:5), (b) Thr-Ile-Ser-Thr-Ser-Gly-Gly-Arg-Thr-Tyr-Tyr-Arg-Asp-Ser-Val-Lys-Gly (SEQ ID NO:6), (c) Phe Arg Gln Tyr Ser Gly Gly Phe Asp Tyr (SEQ ID NO:7) and three “light chain” CDRs, typically on a different protein chain from the heavy chain CDRs (except in the case of certain antibody variants/fragments as described elsewhere herein), comprising, consisting, or consisting essentially of the sequences (d) Thr Leu Ser Ser Gly Asn Ile Glu Asn Asn Tyr Val His (SEQ ID NO:8), (e) Asp Asp Asp Lys Arg Pro Asp (SEQ ID NO:9), and (f) His Ser Tyr Val Ser Ser Phe Asn Val (SEQ ID NO:10).

In another particular exemplar aspect, the invention provides fusion proteins comprising an anti-CD3 antibody portion comprising a heavy chain portion comprising, consisting, or consisting essentially of the sequence Met Gly Leu Ser Gln Val Gln Leu Gln Glu Ser Gly Pro Gly Leu Val Lys Leu Pro Gly Thr Leu Ser Leu Thr Cys Ala Val Ser Gly Gly Ser Ile Ser Ser Ser Asn Trp Trp Ser Trp Val Arg Gln Pro P Gly Lys Gly Leu Glu Trp Ile Gly Gln Ile Ser His Ser Gly Ser Thr Asn Tyr Asn Pro Ser Leu Lys Ser Arg Val Thr Ile Ser Ala Asp Thr Ser Lys Asn Gln Phe Ser Leu Lys Val Asn Ser Val Thr Ala Ala Asp Thr Ala Val Tyr Tyr Cys Ala Arg Arg Asn Tyr Asp Ile Trp Ser Gly Gly Asp Gly Pro Phe Asp Tyr Trp Gly Gln Gly Thr Leu Val Thr Val Ser (SEQ ID NO:11) and a light chain portion comprising, consisting, or consisting essentially of a sequence Met Glu Phe Gly Leu Ser Trp Val Phe Leu Val Ala Ile Leu Glu Gly Val His Cys Glu Val Gln Leu Val Glu Ser Gly Gly Gly Leu Val Gln Pro Gly G Ser Leu Arg Leu Ser Cys Ala Ala Ser Gly Phe Thr Phe Asp Asp Tyr Tyr Leu Asn Trp Ile Arg Gln Ala Pro Gly Lys Gly Leu Glu Trp Val Ala Asn Ile Lys Arg Asp Gly Arg Glu Lys Tyr Tyr Val Asp Ser Val Glu Gly Arg Phe Thr Ile Ser Arg Asp Asn Ala Gln Asn Ser Leu Phe Leu Asn Leu Asn Ser Leu Arg Ala Glu Asp Thr Ala Val Tyr Tyr Cys Ala Arg Arg Ser Gly Gly Thr Thr Gly Tyr Phe Asp Leu Trp Gly Arg Gly Thr Leu Val Thr Val Ser (SEQ ID NO:12).

The basic properties of a functional antibody sequence typically include the specific binding properties thereof. Such basic properties may be shared by several sequences, which, in the context of an inventive fusion protein, may form novel combinations with other amino acid sequences.

Additional anti-CD3 antibody sequences, portions of which may be directly used as an effector lymphocyte activating receptor-binding portion, or that may be modified to produce functional variants for inclusion in an antibody portion, are recorded under GenBank Accession Nos. AAC28461 and AAC28462 (related light chain and heavy chain precursors, respectively); AAA39159 and AAA39272 (related light chain and heavy chain variable sequences, respectively); AAB81028 and AAB81027 (related heavy chain and light chain variable sequences); CAB63951; CAC10847; AAC62751; AAC28464; AAB81026; AAB81025; and CAB65246; and Leo et al., Proc. Natl. Acad. Sci. U.S.A. 84 (5), 1374-1378 (1987) and Bruenke et al., Br J. Haematol. 2004 April; 125(2):167-79.

In another exemplary aspect, the invention provides fusion proteins comprising anti-body portions that are specific for CD16. In one aspect, the invention provides fusion proteins comprising a single antibody portion, which single antibody portion is specific for CD16. In another aspect, the invention provides fusion proteins with multiple antibody portions, each of which being specific for CD16. In yet another aspect, the invention provides fusion proteins comprising multiple antibody portions, only one of which is specific for CD16, the other being specific for another protein or the fusion protein being characterized by comprising at least one bispecific or higher-order multispecific antibody portion wherein one of the specificities of the antibody portion is for CD16.

In one exemplary aspect the invention provides fusion proteins comprising a CD16-specific antibody portion that comprises an anti-CD16 heavy chain variable region sequence that comprises, consists, or consists essentially of the sequence Met Asp Arg Leu Thr Ser Ser Phe Leu Leu Leu Ile Val Pro Ala Tyr Val Leu Ser Gln Val Thr Leu Lys Glu Ser Gly Pro Gly Ile Leu Gln Pro Ser Gln Thr Leu Ser Leu Thr Cys Ser Phe Ser Gly Phe Ser Leu Arg Thr Ser Gly Met Gly Val Gly Trp Ile Arg Gln Pro Ser Gly Lys Gly Leu Glu Trp Leu Ala His Ile Trp Trp Asp Asp Asp Lys Arg Tyr Asn Pro Ala Leu Lys Ser Arg Leu Thr Ile Ser Lys Asp Thr Ser Ser Asn Gln Val Phe Leu Lys Ile Ala Ser Val Asp Thr Ala Asp Thr Ala Thr Tyr Tyr Cys Ala Gln Ile Asn Pro Ala Trp Phe Ala Tyr Trp Gly Gln Gly Thr Leu Val Thr Val Ser Ala (SEQ ID NO:13) and a anti-CD16 light chain variable region sequence that comprises, consists, or consists essentially of the sequence Met Glu Thr Asp Thr Ile Leu Leu Trp Val Leu Leu Leu Trp Val Pro Gly Ser Thr Gly Asp Thr Val Leu Thr Gln Ser Pro Ala Ser Leu Ala Val Ser Leu Gly Gln Arg Ala Thr Ile Ser Cys Lys Ala Ser Gln Ser Val Asp Phe Asp Gly Asp Ser Phe Met Asn Trp Tyr Gln Gln Lys Pro Gly Gln Pro Pro Lys Leu Leu Ile Tyr Thr Thr Ser Asn Leu Glu Ser Gly Ile Pro Ala Arg Phe Ser Ala Ser Gly Ser Gly Thr Asp Phe Thr Leu Asn Ile His Pro Val Glu Glu Glu Asp Thr Ala Thr Tyr Tyr Cys Gln Gln Ser Asn Glu Asp Pro Tyr Thr Phe Gly Gly Gly Thr Lys Leu Glu Ile Lys (SEQ ID NO:14).

In another illustrative aspect, the invention provides a fusion protein comprising a CD16-specific antibody portion that comprises a heavy chain variable sequence comprising, consisting, or consisting essentially of the sequence Gln Val Thr Leu Lys Glu Ser Gly Pro Gly Ile Leu Gln Pro Ser Gln Thr Leu Ser Leu Thr Cys Ser Phe Ser Gly Phe Ser Leu Arg Thr Ser Gly Met Gly Val Gly Trp Ile Arg Gln Pro Ser Gly Lys Gly Leu Glu Trp Leu Ala His Ile Trp Trp Asp Asp Asp Lys Arg Tyr Asn Pro Ala Leu Lys Ser Arg Leu Thr Ile Ser Lys Asp Thr Ser Ser Asn Gln Val Phe Leu Lys Ile Ala Ser Val Asp Thr Ala Asp Thr Ala Thr Tyr Tyr Cys Ala Gln Ile Asn Pro Ala Trp Phe Ala Tyr Trp Gly Gln Gly Thr Leu Val Thr Val Ser Ala (SEQ ID NO:15) and a light chain variable sequence comprising, consisting, or consisting essentially of the sequence Ile Val Leu Thr Gln Ser Pro Ala Ser Leu Ala Val Ser Leu Gly Gln Arg Ala Thr Ile Ser Cys Lys Ala Ser Gln Ser Val Asp Phe Asp Gly Asp Ser Phe Met Asn Trp Tyr Gln Gln Lys Pro Gly Gln Pro Pro Lys Leu Leu Ile Tyr Thr Thr Ser Asn Leu Glu Ser Gly Ile Pro Ala Arg Phe Ser Ala Ser Gly Ser Gly Thr Asp Phe Thr Leu Asn Ile His Pro Val Glu Glu Glu Asp Thr Ala Thr Tyr Tyr Cys Gln Gln Ser Asn Glu Asp Pro Tyr Thr Phe Gly Gly Gly Thr Lys Leu Glu Leu Lys Arg (SEQ ID NO:16).

As with other specific and exemplary sequences provided herein, variants and functional fragments of the particular CD3-specific and CD16-specific sequences also or alternatively may be used as antibody portion components in fusion proteins of the invention.

In another particular aspect, the invention provides a fusion protein comprising an antibody portion that corresponds to at least a portion of an antibody against a T cell coreceptor. An example of a suitable T cell coreceptor is the CD28 coreceptor. Antibodies against the CD28 coreceptor, methods of producing the same, and related methods and compositions are known in the art (see, e.g., US Patent Publication Nos. 20040092718, 20030219446, and 20030170232).

In another aspect, the invention provides fusion proteins that comprise an antibody portion that corresponds to at least a portion of an antibody against a natural killer T (NKT) cell surface protein or a functional variant of such an antibody portion. Natural Killer T cells (NKT cells) are a unique subset of lymphocytes that express natural killer (NK) and T cell receptors (TCR). NKT cells generally display αβ TCRs and commonly one or more NK cell receptors. NKT cells can be characterized by the presence of various cell surface molecules (various proposals for subsets of NKT cells have been made—see, e.g., Kronenberg et al., Nat. Rev. Immunol., 2:557-568 (2002) and Godfrey et al., Nat. Rev. Immunol., 4:231-237 (2004)), such as NK1.1 or NKR-P1A (CD161) and a TCR. Many NKT cells can be characterized as comprising a limited repertoire of TCRs (Vα14/Jα18 paired with Vβ8.2, Vβ7 or Vβ2). Thus, fusion proteins targeting a large set of NKTs can be obtained by inclusion of an anti-body portion that corresponds to an antibody that binds to such TCRs. The sequences of several NKT receptors are known (see, e.g., Lanier et al., J. Immunol. 153 (6), 2417-2428 (1994) and GenBank Accession No. 138700), such that antibodies against NKT cell receptors can readily be obtained using known methods exemplified by techniques described elsewhere herein. Examples of NKT cell receptor-specific antibodies are known in the art (see, e.g., Maruoka et al., Biochem Biophys Res Commun. 1998 Jan. 14; 242(2):413-8).

In another aspect, the invention provides fusion proteins that comprise an antibody portion that corresponds to at least an antigen-binding portion of an antibody against CD3, CD4, CD8, CD16, CD28, CD16, NKp30, NKp44, or NKp46.

In yet another aspect, the invention provides fusion proteins comprising an antibody portion that corresponds to an antibody that is specific for an NK cell receptor. In a more particular aspect, the first portion is derived from an antibody that is specific for a NK cell activity-modulating receptor—i.e., a receptor that modulates the activity of an associated NK cell).

Most NK cell activity-modulating receptors belong to one of two classes of proteins: the immunoglobulin (Lg)-like receptor superfamily (IgSF) or the C-type lectin-like receptor (CTLR) super family (see, e.g., Radaev and Sun, Annu. Rev. Biomol. Struct. 2003 32:93-114). However, other forms of such receptors are known. The structures of a number of NK cell activity-modulating receptor have been elucidated (Id.). To better illustrate the invention, types of well understood NK cell activity-modulating receptors with reference to particular examples thereof, are described here. However, several additional NK cell activity-modulating receptor s are known besides those receptors explicitly described here (see, e.g., Farag et al., Expert Opin. Biol. Ther. 3(2):237-250).

Activity-modulating receptors can be divided into activating and inhibitory receptors. Many NK cell activating receptors belong to the Ig superfamily (IgSF) (such receptors also may be referred to as Ig-like receptors herein). Activating Ig-like NK receptors include, e.g., CD2, CD16, CD69, DNAX accessory molecule-1 (DNAM-1), 2B4, NK1.1; activating killer immunoglobulin (Ig)-like receptors (KIRs); ILTs/LIRs; and natural cytotoxicity receptors (NCRs) such as NKp44, NKp46, and NKp30, Several other NK cell activating receptors belong to the CLTR superfamily (e.g., NKRP-1, CD69; CD94/NKG2C and CD94/NKG2E heterodimers, NKG2D homodimer, and in mice, activating isoforms of Ly49 (such as Ly49A-D)). Still other NK cell activating receptors (e.g., LFA-1 and VLA-4) belong to the integrin protein superfamily and other activating receptors may have even other distinguishable structures. Many NK cell activating receptors possess extracellular domains that bind to MHC-I molecules, and cytoplasmic domains that are relatively short and lack the inhibitory (ITIM) signaling motifs characteristic of inhibitory NK receptors. The transmembrane domains of these receptors typically include a charged amino acid residue that facilitates their association with signal transduction-associated molecules such as CD3ζ, FcεRIγ, DAP12, and DAP10 (2B4, for example, appears to be an exception to this general rule), which contain short amino acid sequences termed an ‘immunoreceptor tyrosine-based activating motif’ (ITAMs) that propagate NK cell-activating signals. Receptor 2B4 contains 4 so-called ITSM motifs (Immunoreceptor Tyrosine-based Switch Motifs) in its cytoplasmic tail; ITSM motifs can also be found in the NK cell activating receptors CS1/CRACC and NTB-A.

Specific examples of activating NK cell receptors that an antibody portion may specifically bind and activate include 2B4; NKR-P1A; NKR-P1B; NKR-P1C; NKG2C; NKG2D; NKG2E; CD16, CD244, CD69; FcεRIII; activating KIRs such as p50.1 (KIR2DS1), p50.2, and p50.3; natural cytotoxicity receptors (NCRs) such as NKp46, NKp30, and NKp44; activating Ly49 molecules (e.g., Ly49D, Ly49H); and ILTs/LIRs (a number of additional NK receptors are known in the art and various additional examples may be provided elsewhere herein). In one aspect, the invention provides fusion proteins that comprise an antibody portion that is specific for an activating NCR. In another aspect, the invention provides fusion proteins that comprise an antibody portion that is specific for at least one NK CTLR or a portion thereof (e.g., CD94/NKG2C, CD94/NKG2E, NKG2D, or an activating isoform of Ly49). In a specific embodiment, the antibody portion does not bind NKG2D.

Activating isoforms of human KIRs (e.g., KIR2DS and KIR3DS) and murine Ly-49 proteins (e.g., Ly-49D and Ly-49H) are expressed by some NK cells and may be advantageous targets for antibody portions. These activating KIR receptors differ from their inhibitory counterparts by lacking inhibitory motifs (ITIMs) in their relatively shorter cytoplasmic domains and possessing a charged transmembrane region that associates with signal-transducing polypeptides, such as disulfide-linked dimers of DAP12. The most common Caucasian human haplotype, the “A” haplotype (frequency of ˜47-59%), contains only one activating KIR gene (KIR2DS4). Thus, in one aspect, the invention provides fusion proteins that comprise an antibody portion that is specific for KIR2DS4. The remaining “B” haplotypes are very diverse and contain 2-5 activating KIR loci (including KIR2DS1,-2DS2, -2DS3, and 2DS5). Fusion proteins comprising an antibody portion that binds and activates one or more of each of these types of KIRs (and/or one or more of these types of KIRs in combination with KIR2DS4) are further features of the invention. In a particular aspect, the invention provides fusion proteins comprising one or more antibody portions that bind and activate KIR2DS4 and/or KR2DS3.

Activating KIRs have been characterized (see, e.g., GenBank Accession Nos. NP_{—}036446, NP_{—}839942, P43632, AAR16203, AAR16204, AAR26325, CAD10378, CAD10379, CAF05810, and CAF05811, with respect to KIR2DS4 proteins; Q14954, NP_{—}055327, AAP33625, and AAB95319, with respect to KIR2DS1 proteins; NP_{—}055034, NP_{—}036444, NP_{—}937758, NP_{—}003323, CAC40718, CAC40717, P43631, AAR16202, AAR16201, with respect to KIR2DS2 proteins; NP_{—}036445 and AAB95320, with respect to KIR2DS3 proteins; and NP_{—}055328 and Q14953, with respect to KIR2DS5 proteins (other examples also are known)). Accordingly, antibodies can be generated against these receptors, screened for receptor specificity; activating KIR-specific sequences therefrom can be identified therefrom; and functional portions of such antibody sequences can incorporated (or functional variants thereof generated and incorporated) in a fusion protein of the invention using standard techniques. Antibodies against such proteins also are known and sequences therefrom may be used in the construction of fusion proteins of the invention. See, e.g., Vitale et al., Int Immunol. 2004 October; 16(10):1459-66; Shin et al., Hybridoma. 1999 December; 18(6):521-7; and US Patent Publication No. 20030232051).

In another aspect, the invention provides fusion proteins comprising an antibody portion that is specific for a activating non-KIR NK cell receptor (NKCR), such as a natural cytotoxicity receptor (NCR) or, for example, NKG2D. Other examples of such targets include NKG2C/CD94, and NKRP1. These and related proteins, methods, and principles have been characterized, such that antibodies can be readily generated against these and similar proteins; functional antibodies can be selected for specificity, and other desired properties; portions of such antibodies can be sequenced; and functional sequences thereof (or variant sequences of such antibody sequences) may be inserted into a fusion protein of the invention using standard techniques. Examples of such antibodies also have been described. Reference can be made, in this respect, to, e.g., GenBank Accession Nos. NP_{—}031386 and NP_{—}031386 (with respect to NKG2D proteins); CAA04922, AAG26338, and Q9GME8 (with respect to NKG2C proteins); BAB91332, CAA74663, Q9MZK9, Q9MZ41, AAC50291, CAA03845, BAA24451, Q8 MHY9, and Q13241 (with respect to CD94 proteins); see also US Patent Publication Nos. 20040115198, 20040038339, 20040072256, 20050130130, 20040038894, and 20030095965 in connection with such proteins, methods, principles, and antibodies.

In another aspect, the invention provides fusion proteins comprising an antibody portion that is specific for an NCR (e.g., NKp30, NKp46, or NKp44). A number of NCR proteins have been described, such that antibodies can be produced against such targets; functional antibodies with desired characteristics (e.g., receptor activation) can be selected; functional portions thereof sequenced; and sequences thereby obtained inserted into an a fusion protein of the invention. Reference may be made, in this respect, to, e.g., GenBank Accession Nos. BAB78472, CAD56759, AAP13457, and CAC41081 (with respect to NKp30 proteins); CAA04714, AAK63120, AAP33623, CAC41080, and Q8C567 (with respect to NKp46 proteins); and 095944 and CAB39168 (with respect to NKp44 proteins). Antibodies against NCRs also have been described or are known, as are related methods and principles, and such may be directly used in production of fusion proteins (see, e.g., US Patent Publication No. 20040072256 and International Patent Applications WO 2005051973, WO 2005000086, and WO 0136630.

In a more particular aspect, the invention provides fusion proteins comprising an antibody portion comprising sequences that correspond to antibody sequences or functional variants thereof, wherein the antibody portion of the fusion protein lacks a significant portion (e.g., contains less about 50% or less, about 40% or less, about 30% or less, about 20% or less, about 15% or less, about 10% or less, etc.) of the Fc portion of the antibody that it corresponds to or from which it is derived (i.e., that it is most related to in terms of sequence identity in the case of a variant antibody portion).

In another aspect, the invention provides fusion proteins comprising or having only antibody portions that are from non-antibody antibody-like proteins, such as an affibody, an anticalin, etc.

In one aspect, the invention provides fusion proteins comprising an antibody portion that corresponds to a functional portion of an affibody or a variant thereof. Affibodies are a class of small, highly specific, and robust affinity proteins, designed to bind desired target proteins (see, e.g., U.S. Pat. No. 5,831,012). Affibodies typically can be characterized as simple proteins composed of a three-helix bundle based on the scaffold of one of the IgG-binding domains of Protein A. Protein A is a well known surface protein from the bacterium Staphylococcus aureus. This scaffold has excellent features as an affinity ligand and can be designed to bind with high affinity to any given target protein. The domain consists of 58 amino acids, 13 of which are randomized to generate affibody libraries with a large number of ligand variants. Such libraries can consist of a multitude of protein ligands with an identical backbone and variable surface-binding properties. Current libraries (e.g., available through Affibody®, Teknikringen 30, floor 6, Box 700 04, Stockholm SE-10044, Sweden) contain billions of variants. Affibodies are characterized as “robust” in that they typically are able to withstand a broad range of physical conditions, including pH and elevated temperature, as compared to e.g., antibodies. Affibodies typically have a molecular weight of about 6 kDa, compared to the molecular weight of antibodies, which typically is about 150 kDa. In function, affibody molecules mimic antibodies. In spite of the small size of these molecules, the binding site of affibody molecules has been demonstrated to be very similar to that of an antibody. Affibodies advantageously can be produced in bacteria and by chemical synthesis (e.g., combinatorial protein engineering). They also can be effectively coupled to form multimeric constructs. Affibodies can further be conjugated to other molecules to form derivatives and fused to form fusion proteins. Affibodies can be “engineered” to have desired properties (e.g., high specificity and affinity—typically nanomolar level affinity). A specific affibody will thus typically bind only to its target in a wide context of molecules. The small size (only about 60 amino acids), high solubility, ease of further engineering into multifunctional constructs, excellent folding, absence of cysteines, and stable scaffold that can be produced in large quantities using low cost bacterial expression systems, make affibodies powerful capture molecules. Methods and principles relevant to the design (e.g., generation), production, and use of affibodies (including exemplary additional modifications to such molecules), can be found in, e.g., Graslund et al., J. Biotechnol. 99, 41; Nygren et al., Curr Opin Struct Biol 7, 463-469 (1997); Nord et al., Nature Biotechnol 15, 772-777 (1997); Nord et al., Protein Eng 8, 601-608 (1995); Högbom et al., Proc. Natl. Acad. Sci. U.S.A. 100, 3191-3196; Wahlberg et al., Proc. Natl. Acad. Sci. U.S.A. 100, 3185-3190; Ronnmark et al., J. Immunol. Meth. 261, 199-211; Ronnmark et al., J. Immunol. Meth. 281, 149-160; Karlström, et al., J. Anal. Biochem. 295, 22-30; Nord et al., J. Biotechnol. 80, 45-54; Eklund et al., Proteins 48, 454-462 (2002); Gunneriusson et al., Protein Eng 12, 873-878 (1999); Wikman et al., Protein Engineering, Design & Selection (advance access published Jun. 18, 2004); Sandstrom et al., Protein Engineering vol. 16 no. 9 pp. 691-697, 2003; Högbom et al., Curr. Opin. Biotechnol., 15(4):364-373 (2004); and U.S. Pat. No. 6,740,734.

In another exemplary aspect, the invention provides fusion proteins comprising or being limited to having non-antibody derived antibody portion(s) that correspond to a functional portion of a trinectin, monobody, or other binding protein based on a fibronectin scaffold. Trinectins comprise a protein binding scaffold that is based on a domain of fibronectin (the 10th fibronectin type III domain). Because these proteins are derived from naturally occurring, circulating human proteins, immune reactions that would otherwise interfere with therapeutic utility are expected to be minimized. In addition, the low molecular weight and compact structure of the molecule (as compared to antibodies) results in a highly stable structure that may enhance target antigen binding. Trinectins are described in, e.g., Xu et al., Chem. Biol. 9:933, 2002 and International Patent Application WO 02/32925. Such molecules are commercially available from Phylos, Inc. (USA) now owned by Compound Therapeutics (Waltham, Mass., USA). Fibronectin type III domain (Fn3) monobodies are similar molecules as are monobodies. These proteins are usually characterized as a polypeptide comprising least two Fn3 β-strand domain sequences with a loop region sequence linked between each Fn3 β-strand domain sequence, wherein the loop region comprises target binding sequences. Examples of such monobodies and related principles, compositions, and methods are described further in, e.g., U.S. Pat. Nos. 6,673,901; 6,703,199; and 6,462,189; Koide et al., (1998), J. Mol. Biol. 284, 1141-1151; Batori et al., Protein Eng. 2002 December; 15(12):1015-20; Karatan et al., Chem. Biol. 2004 June; 11 (6):835-44; and Koide et al., (2001) Biochemistry 40, 10326-10333.

Another type of non-antibody antibody-like protein is an anticalin. Anticalins, like trinectins, are relatively small proteins (as compared to antibodies) that can be engineered to bind specific targets. Anticalins are based on a lipocalin protein scaffold. Anticalins typically are obtained by modifying a protein of the lipocalin family by amino acid replacement in their natural ligand binding pocket, e.g., using genetic engineering methods. Anticalins typically are small monomeric proteins consisting of only 150 to 190 amino acids. Anticalins can exhibit highly specific binding of small molecules and can penetrate tissues such as solid tumors more efficiently. Anticalins are produced by an entirely in vitro process, making it possible to access targets that are either toxic or non-immunogenic. The pharmacokinetic properties of anticalins can be easily controlled by chemical modifications. Compared to monoclonal antibody therapeutics on the market, anticalins might offer better delivery options, such as enhanced topical, pulmonary, or nasal delivery. Anticalins have two potential fusion termini that can be modified without impacting the binding site, thus multispecific anticalins and/or other conjugates or fusion proteins such as, for example, immunotoxins can readily be generated. The central element of the anticalin protein architecture is a beta-barrel structure of eight antiparallel strands, which supports four loops at its open end. These loops form the natural binding site of the lipocalins and can be reshaped in vitro by amino acid replacements and other modifications, thus creating novel binding specificities. Using bacterial phagemid display and colony screening techniques, anticalins can be selected from libraries of randomly generated molecules (typically molecules with affinities in the KD values in the low nanomolar range). Anticalins possess high affinity (e.g., low nanomolar or even in the range of about 100 picomolar) and specificity for their prescribed ligands as well as fast binding kinetics, so that their functional properties are similar to those of antibodies. However, anticalins comprise a simple set of four hypervariable loops that can be easily manipulated at the genetic level. Anticalins, related principles, methods, and the like are described further in, e.g., Skerra, A. (2000) Biochim. Biophys. Acta 1482, 337-350; Beste et al. (1999) Proc. Natl. Acad. Sci. USA 96, 1898-1903; Schlehuber et al. (2000) J. Mol. Biol. 297, 1105-1120; Schlehuber et al. (2001) Biol. Chem. 382, 1335-1342; Skerra, A. (2001) Rev. Mol. Biotechnol. 74, 257-275; Skerra, J. Biotechnol. 2001 June; 74(4):257-75; Weiss et al., Chem. Biol. 2000 August; 7(8):R177-84; WO 99016873; and EP1017814.

Various other types of antibody mimetic antibody-like proteins may also be used as antibody portions (either exclusively or non-exclusively). A variety of such antibody-like proteins are known or have been proposed. For example, peptides comprising a synthetic betaloop structure that mimics the second complementarity-determining region (CDR) of MAbs have been proposed and generated. See, e.g., Saragovi et al., Science. 1991 Aug. 16; 253(5021):792-5. Peptide Ab mimetics also have been generated by use of peptide mapping to determine ‘active’ antigen recognition residues, molecular modeling, and a molecular dynamics trajectory analysis, so as to design a peptide mimic containing antigen contact residues from multiple CDRs. See, e.g., Cassett et al., Biochem Biophys Res Commun. 2003 Jul. 18; 307(1):198-205. Additional discussion of related principles, methods, etc., that may be applicable in the context of antibody portions are provided in, e.g., Fassina, Immunomethods. 1994 October; 5(2):121-9.

### Target-Binding Portion

In general, a target-binding portion or membrane protein extracellular domain portion of a fusion protein provided by this invention refers to any one or more amino acid sequences that comprise, consist, or consist essentially of the functional portion of the extracellular domain of a cell membrane protein, or a functional variant thereof, that is capable of binding at least one “second” or “secondary” cell-associated molecule or target (typically a cell-associated protein that serves as a receptor or ligand for the membrane protein that the target-binding protein corresponds or is most related to in terms of sequence identity and other relevant properties). In one aspect, the secondary target is not bound by the antibody portion(s). In other aspects, the secondary target is also or alternatively not bound by the effector-lymphocyte-activating receptor.

The functional portion of an extracellular domain is the portion that is able to impart receptor binding. The receptor binding portion of an extracellular domain may be known or determined by standard techniques. An target-binding protein need not be limited to the extracellular domain of the membrane protein. Thus, transmembrane and/or intracellular sequences of such a protein may be included in a fusion protein of the invention where the presence of such sequences does not deter from the functionality of the fusion protein.

In one aspect, the invention provides fusion proteins comprising an target-binding portion that corresponds to the extracellular domain of a Type II membrane protein or a functional variant of such a membrane protein. As is generally known, “Type II membrane proteins” are generally characterized as proteins that span the membrane of a cell, typically one time, but have (in contrast to Type I proteins) their amino terminus presented on the cytoplasmic side of the cell and the carboxy terminus on the exterior. Type II membrane proteins typically are also characterized by the lack of a cleavable signal sequence in originally expressed form and/or inclusion of relatively long hydrophobic regions that are anchored in the membrane. In addition to Type I membrane proteins, Type II membrane proteins may be distinguished from Type III and Type IV membrane proteins. The use of Type II membrane proteins is advantageous, particularly in ease of construction of the protein. Such proteins may be fused in large part (including sequences outside of the extracellular domain) to anti-body sequences (e.g., a heavy chain sequence of an antibody). Due to the orientation of domains in this type of membrane protein, the target-binding portion may be fused at its N-terminus to the C-terminus of an antibody sequence (directly or indirectly) that forms an antibody portion (e.g., the heavy chain of an anti-effector lymphocyte activating receptor antibody), such that very little modification of the membrane protein sequence is required in generating a functional fusion protein (given that the functional C-terminus of the membrane protein and N-terminus of the antibody heavy chain are readily available to bind their respective targets).

In a more particular aspect, the invention provides fusion proteins comprising a target-binding portion that can be characterized as corresponding to (or comprising a portion corresponding to) at least a functional portion of a disulfide-linked C-type lectin receptor domain protein.

In another particular aspect, the invention provides fusion proteins comprising a target-binding portion that corresponds to (or that comprises a portion that corresponds to) at least a functional portion of a C-type lectin receptor (CTLR), which may be, for example, a disulfide-linked CTLR.

In a particular and advantageous aspects, the invention provides fusion proteins comprising a target-binding protein that, in addition to any of the foregoing, may also or alternatively be characterized as a receptor or a ligand for a cell-associated molecule that is presented on or expressed by cells associated with a disease state normally regulated by effector lymphocytes, such as cancer, viral infection, or the like. Thus, for example, a typical target-binding protein may correspond to a functional portion of a receptor for cell stress-associated molecules, such as a MIC molecule (e.g., MIC-A or MIC-B) or a ULBP (e.g. Rae-1, H-60, ULBP2, ULBP3, HCMV UL18, or Rae-1β) or a pathogen-associated molecule such as a viral hemagglutinin. In one such aspect, the invention provides fusion proteins comprising a target-binding portion that corresponds to a functional portion of a NK cell receptor or a functional variant thereof. Such an NK cell receptor may be, e.g., an immunoglobulin super family (IgSF) receptor. An NK cell receptor may be a natural cytotoxicity receptor (NCR). A NK cell receptor alternatively also may be an activating KIR. In one exemplary aspect, the invention provides fusion proteins comprising a target-binding portion that corresponds to a functional portion of an NK cell receptor selected from NKG2D, NKG2A/CD94, CD69, NKG2C/CD94, NKG23/CD94, NKG2F/CD94, LLT1, AICL, CD26, and NKRP1 (CD161), or a functional variant thereof.

In one aspect, a target-binding portion is not and/or does not bind a MHC molecule.

In another aspect, the invention provides fusion proteins wherein a target-binding protein corresponds to a functional portion of an inhibitory NK cell receptor or a functional variant thereof. For example, in one aspect, the invention provides fusion proteins comprising a target-binding portion that corresponds to a functional portion of NKG2A/CD94, an inhibitory KIR, an LIR (leukocyte inhibitory receptor), or FcγRIIB.

In one exemplary aspect, the invention provides fusion proteins that comprise a target-binding portion that corresponds to at least a functional portion of a NKG2D extracellular domain having or consisting essentially of the sequence Leu Phe Asn Gln Glu Val Gln Ile Pro Leu Thr Glu Ser Tyr Cys Gly Pro Cys Pro Lys Asn Trp Ile Cys Tyr Lys Asn Asn Cys Tyr Gln Phe Phe Asp Glu Ser Lys Asn Trp Tyr Glu Ser Gln Ala Ser Cys Met Ser Gln Asn Ala Ser Leu Leu Lys Val Tyr Ser Lys Glu Asp Gln Asp Leu Leu Lys Leu Val Lys Ser Tyr His Trp Met Gly Leu Val His Ile Pro Thr Asn Gly Ser Trp Gln Trp Glu Asp Gly Ser Ile Leu Ser Pro Asn Leu Leu Thr Ile Ile Glu Met Gln Lys Gly Asp Cys Ala Leu Tyr Ala Ser Ser Phe Lys Gly Tyr Ile Glu Asn Cys Ser Thr Pro Asn Thr Tyr Ile Cys Met Gln Arg Thr Val (SEQ ID NO:17) (see Ho et al., Proc. Natl. Acad. Sci. USA, Vol. 95, Issue 11, 6320-6325, 1998) or a functional variant thereof. See also, Pende et al., J. Exp. Med. 190 (10), 1505-1516 (1999). An exemplary functional portion comprises the sequence

In another exemplary aspect, the invention provides fusion proteins that comprise a target-binding portion that corresponds to at least a functional portion of an NKp30 extracellular domain having or consisting essentially of the sequence Ile Trp Val Ser Gln Pro Pro Glu Ile Arg Ala Gln Glu Gly Thr Thr Ala Ser Leu Pro Cys Ser Phe Asn Ala Ser Arg Gly Lys Ala Ala Ile Gly Ser Ala Thr Trp Tyr Gln Asp Lys Val Ala Pro Gly Met Glu Leu Ser Asn Val Thr Pro Gly Phe Arg Gly Arg Val Ala Ser Phe Ser Ala Ser Gln Phe Ile Arg Gly His Lys Ala Gly Leu Leu Ile Gln Asp Ile Gln Ser His Asp Ala Arg Ile Tyr Val Cys Arg Val Glu Val Leu Gly Leu Gly Val Gly Thr Gly Asn Gly Thr Arg Leu Val v Glu Lys Glu Pro p Gln Gln Ala Ser Asn Ala Glu Pro Glu Arg Ala Ala Tyr Thr Ser (SEQ ID NO:18) or a functional variant thereof.

In still another illustrative aspect, the invention provides fusion proteins that comprise a target-binding portion that corresponds to at least a functional portion of a NKp46 extracellular domain having or consisting essentially of the sequence Thr Leu Pro Lys Pro Phe Ile Trp Ala Glu Pro His Phe Met Val Pro Lys Glu Lys Gln Val Thr Ile Cys Cys Gln Gly Asn Tyr Gly Ala Val Glu Tyr Gln Leu His Phe Glu Gly Ser Leu Phe Ala Val Asp Arg Pro Lys Pro Pro Glu Arg Ile Asn Lys Val Lys Phe Tyr Ile Pro Asp Met Asn Ser Arg Met Ala Gly Gln Tyr Ser Cys Ile Tyr Arg Val Gly Glu Leu Trp Ser Glu Pro Ser Asn Leu Leu Asp Leu Val Val Thr Glu (SEQ ID NO:19) or a functional variant thereof.

In a further exemplary variation, the invention provides fusion proteins that comprises a target-binding portion that corresponds to at least a functional portion of a NKp44 extracellular domain having or consisting essentially of the sequence Gln Ser Lys Ala Gln Val Leu Gln Ser Val Ala Gly Gln Thr Leu Thr Val Arg Cys Gln Tyr Pro Pro Thr Gly Ser Leu Tyr Glu Lys Lys Gly Trp Cys Lys Glu Ala Ser Ala Leu Val Cys Ile Arg Leu Val Thr Ser Ser Lys Pro Arg Thr Met Ala Trp Thr Ser Arg Phe Thr Ile Trp Asp Asp Pro Asp Ala Gly Phe Phe Thr Val Thr Met Thr Asp Leu Arg Glu Glu Asp Ser Gly His Tyr Trp Cys Arg Ile Tyr Arg Pro Ser Asp Asn Ser Val Ser Lys Ser Val Arg Phe Tyr Leu Val Val Ser Pro Ala Ser Ala Ser Thr Gln Thr Pro Trp Thr Pro Arg Asp Leu Val Ser Ser Gln Thr Gln Thr Gln Ser Cys Val Pro Pro Thr Ala Gly Ala Arg Gln Ala Pro Glu Ser Pro Ser Thr Ile Pro Val Pro Ser Gln Pro Gln Asn Ser Thr Leu Arg Pro Gly Pro Ala Ala Pro Ile Ala (SEQ ID NO:20) or a functional variant thereof.

In an additional illustrative aspect, the invention provides fusion proteins comprising a target-binding portion that corresponds to at least a functional portion of a CD94 extracellular domain having or consisting essentially of the sequence Lys Asn Ser Phe Thr Lys Leu Ser Ile Glu Pro Ala Phe Thr Pro Gly Pro Asn Ile Glu Leu Gln Lys Asp Ser Asp Cys Cys Ser Cys Gln Glu Lys Trp Val Gly Tyr Arg Cys Asn Cys Tyr Phe Ile Ser Ser Glu Gln Lys Thr Trp Asn Glu Ser Arg His Leu Cys Ala Ser Gln Lys Ser Ser Leu Leu Gln Leu Gln Asn Thr Asp Glu Leu Asp Phe Met Ser Ser Ser Gln Gln Phe Tyr Trp Ile Gly Leu Ser Tyr Ser Glu Glu His Thr Ala Trp Leu Trp Glu Asn Gly Ser Ala Leu Ser Gln Tyr Leu Phe Pro Ser Phe Glu Thr Phe Asn Thr Lys Asn Cys Ile Ala Tyr Asn Pro Asn Gly Asn Ala Leu Asp Glu Ser Cys Glu Asp Lys Asn Arg Tyr Ile Cys Lys Gln Gln Leu Ile (SEQ ID NO:21) or a functional portion thereof.

### Variant or Derivatized Fusion Protein Portions

As discussed elsewhere herein, functional variants of sequences obtainable from a given source, such as a known antibody or receptor, can be used in or as antibody portion and target-binding portion components of the inventive fusion proteins. A “functional variant” of a protein, such as an antibody, or an amino acid sequence, domain, or other portion thereof, such as an antigen-binding portion of an antibody, refers to a protein, sequence, or portion that differs from a reference protein, sequence, or portion by one or more amino acid residue substitutions, additions, insertions, and/or deletions, but which at least substantially retains some (and desirably most or even all) of the functional attributes of the protein (in the case of antibody sequences the relevant functional attribute typically is binding to the same target with an affinity that is sufficient for the desired purpose). A variant is significantly similar in terms of sequence identity with (e.g., exhibits at least about 40%, typically at least about 50%, more typically at least about 60%, even more typically at least about 70%, commonly at least about 80%, frequently as at least about 85%, such as at least about 90%, 95%, or more identity) and usually in possession of other similar physiochemical properties to at least one (referenced) protein or amino acid sequence (which may be referred to as the “parent,” which typically is a naturally occurring (“wild-type”) molecule or molecule component).

Advantageous sequence changes with respect to a parent sequence that frequently are sought in the production of variants are those that (1) reduce susceptibility to proteolysis, (2) reduce susceptibility to oxidation, (3) alter binding affinity of the variant sequence (typically desirably increasing affinity), and/or (4) confer or modify other physicochemical or functional properties on the associated variant/analog peptide. The skilled artisan will be aware of these and other factors in the design, production, and selection of variants In the context of antibody CDR variants, for example, it is typically desired that residues required to support and/or orientate the CDR structural loop structure(s) are retained; that residues which fall within about 10 angstroms of a CDR structural loop (but optionally only residues in this area that also possess a water solvent accessible surface of about 5 angstroms2 or greater) are unmodified or modified only by conservative amino acid residue substitutions; and/or that the sequence is subject to only a limited number of insertions and/or deletions (if any), such that CDR structural loop-like structures are retained in the variant (a description of related techniques and relevant principles is provided in, e.g., Schiweck et al., J Mol. Biol. 1997 May 23; 268(5):934-51; Morea, Biophys Chem. 1997 October; 68(1-3):9-16; Shirai et al., FEBS Lett. 1996 Dec. 9; 399(1-2):1-8; Shirai et al., FEBS Lett. 1999 Jul. 16; 455(1-2):188-97; Reckzo et al., Protein Eng. 1995 April; 8(4):389-95; and Eigenbrot et al., J Mol. Biol. 1993 Feb. 20; 229(4):969-95).

For antibody fragments and portions, the sites of greatest interest for substitution variations typically are the hypervariable regions (or particular CDRs), but variants also or alternatively characterized by one or more framework (FR) alterations may similarly be generated. For example, a substitution or other modification (insertion, deletion, or combination of any thereof) in a framework region or constant domain can be associated with an increase in the half-life of the variant antibody with respect to the parent antibody. A variation in a framework region or constant domain may also be made to alter the immunogenicity of the variant antibody with respect to the parent antibody, to provide a site for covalent or non-covalent binding to another molecule, or to alter such properties as complement fixation. Variations in an antibody variant may be made in each of the framework regions, the constant domain, and/or the variable regions (or any one or more CDRs thereof) in a single variant antibody. Alternatively, variations may be made in only one of the framework regions, the variable regions (or single CDR thereof), or the constant domain in an antibody.

In the design, construction, and/or evaluation of CDR variants, attention typically is paid to the fact that CDR regions can vary to enable a better binding to the epitope. Antibody CDRs typically operate by building a “pocket,” or other paratope structure, into which the epitope fits. If the epitope is not fitting tightly, the antibody may not offer the best affinity. However, as with epitopes, there often are a few key residues in a paratope structure that account for most of this binding. Thus, CDR sequences can vary in length and composition significantly between antibodies for the same peptide. The skilled artisan will recognize that certain residues, such as tyrosine residues (e.g., in the context of CDR-H3 sequences), that are often significant contributors to such epitope binding, are typically desirably retained in a CDR variant.

The phrase “potential amino acid interactions” can be used to refer to contacts or energetically favorable interactions between one or more amino acid residues present in an antigen and one or more amino acid residues which do not exist in a parent antibody but can be introduced therein so as to increase the amino acid contacts between the antigen and an antibody variant comprising those introduced amino acid residue(s). Desirably, antibody variants and antibody fragment variants are associated with increased potential amino acid interactions with a target molecule as compared to their parents. Amino acid interactions of interest can be selected from hydrogen bonding interactions, van der Waals interactions, and/or ionic interactions.

Typically, in a variant of an antibody portion, less than about 10, such as less than about 5, such as 3 or less amino acid variations (differences by way of the above-described methods, e.g., substitution) are present in either the VH or VL regions of the variant antibody or antibody fragment with respect to a parent antibody or antibody fragment.

Identity in the context of amino acid sequences can be determined by any suitable technique, typically by a Needleman-Wunsch alignment analysis (see Needleman and Wunsch, J. Mol. Biol. (1970) 48:443-453), such as is provided via analysis with ALIGN 2.0 using the BLOSUM50 scoring matrix with an initial gap penalty of −12 and an extension penalty of −2 (see Myers and Miller, CABIOS (1989) 4:11-17 for discussion of the global alignment techniques incorporated in the ALIGN program). A copy of the ALIGN 2.0 program is available, e.g., through the San Diego Supercomputer (SDSC) Biology Workbench. Because Needleman-Wunsch alignment provides an overall or global identity measurement between two sequences, it should be recognized that target sequences which may be portions or sub-sequences of larger peptide sequences may be used in a manner analogous to complete sequences or, alternatively, local alignment values can be used to assess relationships between subsequences, as determined by, e.g., a Smith-Waterman alignment (J. Mol. Biol. (1981) 147:195-197), which can be obtained through available programs (other local alignment methods that may be suitable for analyzing identity include programs that apply heuristic local alignment algorithms such as FastA and BLAST programs). Further related methods for assessing identity are described in, e.g., International Patent Application WO 03/048185. The Gotoh algorithm, which seeks to improve upon the Needleman-Wunsch algorithm, alternatively can be used for global sequence alignments. See, e.g., Gotoh, J. Mol. Biol. 162:705-708 (1982).

Typically, variants differ from “parent” sequences mostly through conservative substitutions; e.g., at least about 35%, about 50% or more, about 60% or more, about 70% or more, about 75% or more, about 80% or more, about 85% or more, about 90% or more, about 95% or more (e.g., about 65-99%) of the substitutions in the variant are conservative amino acid residue replacements. In the context of this invention, conservative substitutions can be defined by substitutions within the classes of amino acids reflected in one or more of the following three tables:

TABLE 1
Amino Acid Residue Classes for Conservative Substitutions

| Amino Acid Class | Amino Acid Residues |
|---|---|
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

TABLE 2
Alternative Conservative Amino Acid Residue Substitution Groups

| | | | |
|---|---|---|---|
| 1 | Alanine (A) | Serine (S) | Threonine (T) |
| 2 | Aspartic acid (D) | Glutamic acid (E) | |
| 3 | Asparagine (N) | Glutamine (Q) | |
| 4 | Arginine (R) | Lysine (K) | |
| 5 | Isoleucine (I) | Leucine (L) | Methionine (M) |
| 6 | Phenylalanine (F) | Tyrosine (Y) | Tryptophan (W) |

TABLE 3
Alternative Physical and Functional Classifications
of Amino Acid Residues

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, |
| | R, T, V, W, and Y |
| Negatively charged residues | D and E |
| Polar residues | C, D, E, H, K, N, |
| | Q, R, S, and T |
| Small residues | A, C, D, G, N, P, |
| | S, T, and V |
| Very small residues | A, G, and S |
| Residues involved in | A, C, D, E, G, H, K, |
| turn formation | N, Q, R, S, P, and T |
| Flexible residues | E, Q, T, K, S, G, |
| | P, D, E, and R |

More conservative substitutions groupings include: valine-leucine-isoleucine, phenylalanine-tyrosine, lysine-arginine, alanine-valine, and asparagine-glutamine. Additional groups of amino acids can also be formulated using the principles described in, e.g., Creighton (1984) PROTEINS: STRUCTURE AND MOLECULAR PROPERTIES (2d Ed. 1993), W.H. Freeman and Company. In some instances it can be useful to further characterize substitutions based on two or more of such features (e.g., substitution with a “small polar” residue, such as a Thr residue, can represent a highly conservative substitution in an appropriate context).

Substantial changes in function can be made by selecting substitutions that are less conservative than those shown in the defined groups, above. For example, non-conservative substitutions can be made which more significantly affect the structure of the peptide in the area of the alteration, for example, the alpha-helical, or beta-sheet structure; the charge or hydrophobicity of the molecule at the target site; or the bulk of the side chain. The substitutions which generally are expected to produce the greatest changes in the peptide's properties are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine. Accordingly, these and other nonconservative substitutions can be introduced into peptide variants where significant changes in function/structure is desired and such changes avoided where conservation of structure/function is desired.

Those skilled in the art will be aware of additional principles useful in the design and selection of peptide variants. For example, residues in surface positions of a peptide typically a strong preference for hydrophilic amino acids. Steric properties of amino acids can greatly affect the local structures that a protein adopts or favors. Proline, for example, exhibits reduced torsional freedom that can lead to the conformation of the peptide backbone being locked in a turn and with the loss of hydrogen bonding, often further resulting in the residue appearing on a surface loop of a protein. In contrast to Pro, Gly has complete torsional freedom about a main peptide chain, such that it is often associated with tight turns and regions buried in the interior of the protein (e.g., hydrophobic pockets). The features of such residues often limit their involvement in secondary structures. However, residues typically involved in the formation of secondary structures are known. For example, residues such as Ala, Leu, and Glu (amino acids without much bulk and/or polar residues) typically are associated with alpha-helix formation, whereas residues such as Val, Ile, Ser, Asp, and Asn can disrupt alpha helix formation. Residues with propensity for beta-sheet structure formation/inclusion include Val and Ile and residues associated with turn structures include Pro, Asp, and Gly. The skilled artisan can consider these and similar known amino acid properties in the design and selection of suitable peptide variants, such that suitable variants can be prepared with only routine experimentation.

Desirably, conservation in terms of hydropathic/hydrophilic properties also is substantially retained in a variant peptide as compared to a parent peptide (e.g., the hydropathic score of the parent, in terms of individual residues and/or overall, is at least about 50%, at least about 60%, at least about 70%, at least about 75%, at least about 80%, at least about 85%, at least about 90%, at least about 95%, or more (e.g., about 65-99%) retained in the variant sequence). Methods for assessing the conservation of the hydropathic character of residues/sequences are known in the art and incorporated in available software packages, such as the GREASE program available through the SDSC Biology Workbench (see also, e.g., Kyte and Doolittle et al., J. Mol. Biol. 157:105-132 (1982); Pearson and Lipman, PNAS (1988) 85:2444-2448, and Pearson (1990) Methods in Enzymology 183:63-98 for a discussion of the principles incorporated in GREASE and similar programs).

The retention of similar amino acid residues in a variant as compared to a parent sequence or protein also or alternatively can be measured by a similarity score, as determined by use of a BLAST program (e.g., BLAST 2.2.8 available through the NCBI). Suitable variants typically exhibit at least about 45%, such as at least about 55%, at least about 65%, at least about 75%, at least about 85%, at least about 90%, at least about 95%, or more (e.g., about 70-99%) similarity to the parent peptide.

Variants of antibody sequences can be generated by any one or combination of techniques known in the art. For example, to improve the quality and/or diversity of antibodies against a target, the VL and VH segments of VL/VH pair(s) (or portions thereof) can be randomly mutated, typically at least within the CDR3 region of VH and/or VL, in a process analogous to the in vivo somatic mutation process responsible for affinity maturation of anti-bodies during a natural immune response. Such in vitro affinity maturation can be accomplished by, e.g., amplifying VH and VL regions using PCR primers complimentary to VH CDR3 or VL CDR3 encoding sequences, respectively, which primers typically are “spiked” with a random mixture of the four nucleotide bases at certain positions, such that the resultant PCR products encode VH and VL segments into which random mutations have been introduced thereby resulting (at least in some cases) in the introduction of sequence variations in the VH and/or VL CDR3 regions. Such randomly mutated VH and VL segments can thereafter be re-screened by phage display or other suitable technique for binding to target molecule(s) and advantageous variants analyzed and used to prepare functional variant sequences. Following screening, a nucleic acid encoding a selected antibody, where appropriate, can be recovered from a display package (e.g., from a phage genome) and subcloned into an appropriate vector by standard recombinant techniques. If desired, such an antibody-encoding nucleic acid can be further manipulated to create other antibody forms. To express a recombinant human antibody isolated by screening of a combinatorial library, typically a nucleic acid comprising a sequence encoding the antibody is cloned into a recombinant expression vector and introduced into appropriate host cells (mammalian cells, yeast cells, etc.) under conditions suitable for expression of the nucleic acid and production of the antibody.

A convenient way for generating substitution variants is affinity maturation using phage according to methods known in the art. In order to identify candidate hypervariable region sites for modification, alanine scanning mutagenesis also can be performed to identify hypervariable region residues contributing significantly to antigen binding. Alternatively or additionally, it may be beneficial to analyze a crystal structure of the antigen-antibody complex to identify contact points between the antibody and antigen. Such contact residues and neighboring residues are likely suitable candidates for substitution.

Useful methods for rational design of CDR sequence variants are described in, e.g., International Patent Applications WO 91/09967 and WO 93/16184.

Other methods for generating CDR variants include the removal of nonessential residues as described in, e.g., Studnicka et al., Protein Engineering, Vol 7, 805-814 (1994) (see also Soderlind et al., Immunotechnology. 1999 March; 4(3-4):279-85), CDR walking mutagenesis and other artificial affinity maturation techniques (see, e.g., Journal of Molecular Biology, December 1995; 254(3):392-403), and CDR shuffling techniques wherein typically CDRs are amplified from a diverse set of gene templates optionally comprising synthetic oligonucleotides, the constant regions of the VL, VH, and/or CDRs are amplified, and the various fragments mixed (in single-stranded or double-stranded format) and assembled by polymerase chain reaction (PCR) to produce a set of antibody-fragment encoding gene products carrying shuffled CDR introduced into the master framework, which is amplified using external primers annealing to sites beyond inserted restriction sites to ensure production of full-length products, which are inserted into a vector of choice and used to expressed variant CDR-containing proteins.

Alanine scanning mutagenesis techniques, such as described by, e.g., Cunningham and Wells (1989), Science 244:1081-1085, can be used to identify suitable residues for substitution or deletion in generating antibodies comprising variant VL, VH, or particular CDR sequences, although other suitable mutagenesis techniques also can be applied. Multiple amino acid substitutions also can be made and tested using known methods of mutagenesis and screening, such as those disclosed by Reidhaar Olson and Sauer, Science 241:53 57 (1988) or Bowie and Sauer Proc. Natl. Acad. Sci. USA 86:2152 2156 (1989). Additional techniques that can be used to generate variant antibody sequences include the directed evolution and other variant generation techniques described in, e.g., US 20040009498; Marks et al., Methods Mol. Biol. 2004; 248:327-43 (2004); Azriel-Rosenfeld et al., J Mol. Biol. 2004 Jan. 2; 335(1):177-92; Park et al., Biochem Biophys Res Commun. 2000 Aug. 28; 275(2):553-7; Kang et al., Proc Natl Acad Sci USA. 1991 Dec. 15; 88(24):11120-3; Zahnd et al., J Biol. Chem. 2004 Apr. 30; 279(18):18870-7; Xu et al., Chem. Biol. 2002 August; 9(8):933-42; Border et al., Proc Natl Acad Sci USA. 2000 Sep. 26; 97(20):10701-5; Crameri et al., Nat. Med. 1996 January; 2(1):100-2; and as more generally described in, e.g., International Patent Application WO 03/048185.

Other potentially suitable techniques for preparing novel antibody variant sequences include CDR walking mutagenesis, antibody chain shuffling, “parsimonious mutagenesis” (Balint and Larrick Gene 137:109-118 (1993)), and other affinity maturation techniques (see, e.g., Wu et al. PNAS (USA) 95: 6037-6-42 (1998)). Repertoire cloning procedures also can be useful in the production of variant antibodies (see, e.g., International Patent Application WO 96/33279).

Amino acid sequence variants of an antibody also can be obtained by, for example, introducing appropriate nucleotide changes into an antibody-encoding nucleic acid (e.g., by site directed mutagenesis), by chemical peptide synthesis, or any other suitable technique. Such variants include, for example, variants differing by deletions from, and/or insertions into and/or substitutions of, residues within the amino acid sequences of known antibodies of interest. Any combination of deletions, insertions, and substitutions can be made to arrive at a desired variant, provided that the variant possesses suitable characteristics for practice in the methods of the invention (e.g., a retention of at least a substantial proportion of the parent antibodies affinity, specificity, and/or selectivity with respect to one or more desired epitopes or antigenic determinant regions). Amino acid sequence changes, with respect to a parent antibody, also may alter post-translational processes of the variant antibody with respect to a parent antibody, such as by changing the number or position of glycosylation sites.

Where hypervariable region insertions are made to generate a variant antibody, typical range of lengths of the hypervariable region in question in known antibodies. For example, for the first hypervariable region of a light chain variable domain, insertions can be introduced into the CDR L1 sequence of a parent antibody while retaining a substantially similar and thereby expected appropriate size, which according to Kabat et al., supra, e.g., typically has an overall of about 9-20 (e.g., about 10-17) residues. Similarly, CDR L2 typically has an overall length from about 5-10 residues; CDR L3 typically has a length of about 7-20 residues; CDR H1 typically has a length of about 10-15 residues; CDR H2 typically has a length of about 15-20 residues; and CDR H3 typically has a length of about 6-30 residues (e.g., 3-25 residues). Insertions in the VH region typically are made in CDR H3 and typically near the C-terminal of the domain, such as about residues 97-102 of the parent CDR H3 (e.g., adjacent to, and preferably C-terminal in sequence to, residue number 100 of the parent CDR H3 sequence) using the alignment and numbering as described in Kabat.

Amino acid sequence variations can result in an altered glycosylation pattern in the variant antibody with respect to a parent antibody. By “altered glycosylation” it is meant that one or more carbohydrate moieties found in the parent antibody are not present in the variant, and/or one or more glycosylation sites that are not present in the parent antibody are present in the variant. Also or alternatively, a particular glycosylation site may differ in position in a variant with respect to a parent. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are common recognition sequences for enzymatic attachment of the carbohydrate moiety to the asparagine side chain. Thus, the presence of either of these tripeptide sequences in a polypeptide typically can create a potential glycosylation site. O-linked glycosylation refers to the attachment of sugars such as N-aceylgalactosamine, galactose, or xylose to a hydroxyamino acid, most commonly serine or threonine, although 5-hydroxyproline or 5-hydroxylysine may also be used. Addition of glycosylation sites to the antibody can be conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Typically, amino acid sequence variations, such as conservative substitution variations, desirably do not substantially change the structural characteristics of the parent sequence (e.g., a replacement amino acid should not tend to disrupt secondary structure that characterizes the function of the parent sequence). Examples of art-recognized polypeptide secondary and tertiary structures are described in, e.g., PROTEINS, STRUCTURES AND MOLECULAR PRINCIPLES (Creighton, Ed., W.H. Freeman and Company, New York (1984)); INTRODUCTION TO PROTEIN STRUCTURE (C. Branden and J. Tooze, eds., Garland Publishing, New York, N.Y. (1991)); and Thornton et at. Nature 354:105 (1991). Additional principles relevant to the design and construction of peptide variants is discussed in, e.g., Collinet et al., J Biol Chem 2000 Jun. 9; 275(23):17428-33. Protein structure can be assessed by any number of suitable techniques, such as nuclear magnetic resonance (NMR) spectroscopic structure determination techniques, which are well-known in the art (See, e.g., Wuthrich, NMR of Proteins and Nucleic Acids, Wiley, New York, 1986; Wuthrich, K. Science 243:45-50 (1989); Clore et al., Crit. Rev. Bioch. Molec. Biol. 24:479-564 (1989); Cooke et al. Bioassays 8:52-56 (1988)), typically in combination with computer modeling methods (e.g., by use of programs such as MACROMODEL™, INSIGHT™, and DISCOVER™), to obtain spatial and orientation requirements for structural analogs. Using information obtained by these and other suitable known techniques, structural analogs can be designed and produced through rationally-based amino acid substitutions, insertions, and/or deletions. It also is possible and often desirable that such structural information be used in concert with parent antibody sequence information to design useful antibody variants. Secondary structure comparisons can be made using the EBI SSM program (currently available at http://www.ebi.ac.uk/msd-srv/ssm/). Where coordinates of the variant are known they can be compared by way of alignment/comparison programs such as DALI pair alignment (currently available at http://www.ebi.ac.uk/dali/Interactive.html), TOPSCAN (currently available at http://www.bioinf.org.uk/topscan), COMPARER (currently available at http://wwwcryst.bioc.cam.ac.uk/COMPARER/) PRIDE pair (currently available at http://hydra.icgeb.trieste.it/pride/pride.php?method=pair), PINTS (currently available at http://www.russell.embl.de/pints/), SARF2 (currently available at http://123d.ncifcrf.gov/run2.html), the Structural Alignment Server (currently available at http://www.molmovdb.org/align/), and the CE Calculate Two Chains Server (currently available at http://cl.sdsc.edu/ce/ce_align.html). Ab initio protein structure prediction methods can be applied, if needed, to the variant sequence, such as through the HMM-ROSETTA or MODELLER programs, to predict the structure for comparison with the parent sequence(s) molecule. Where appropriate other structure prediction methods, such as threading methods, also or alternatively can be used, to predict the structure of the variant and/or parent sequence proteins. Additional methods for assessing similarity of peptides in terms of conservative substitutions, hydropathic properties, and similar considerations are described in e.g., International Patent Applications WO 03/048185, WO 03/070747, and WO 03/027246.

The basic properties of “parent” sequences that desirably are retained in variant sequences are similar specificity and suitable affinity for target molecules bound by the parent (retention of at least a substantial proportion of the affinity of the parent sequence for its target, e.g., CD3 in the case of an anti-CD3 antibody). Typically, a suitable affinity for a target falls in the range of about 10⁴ to about 10¹⁰ M⁻¹ (e.g., about 10⁷ to about 10⁹ M⁻¹). A variant antibody portion, for example, may have an average disassociation constant (KD) of about 7×10⁻⁹ M or more with respect to a target (e.g., an activating NK cell receptor), as determined by, e.g., surface plasmon resonance (SPR) screening (such as by analysis with a BIAcore™ SPR analytical device). Typically, variant sequence antibody portions also or alternatively can be characterized by exhibiting target binding with a disassociation constant of less than about 100 nM, less than about 50 nM, less than about 10 nM, about 5 nM or less, about 1 nM or less, about 0.5 nM or less, about 0.1 nM or less, about 0.01 nM or less, or even about 0.001 nM or less. Affinities for target-binding portions desirably are on the order of at least about 50% of that exhibited by the parent membrane protein extracellular domains for the target molecules.

In another aspect, the invention provides derivatized versions of fusion proteins comprising the basic structural features already described herein (in terms of amino acid sequence composition). A “derivative” refers to a protein or amino acid sequence in which one or more of the amino acid residues of the protein have been artificially chemically modified (e.g., by alkylation, acylation, ester formation, amide formation, or other similar type of modification), such as through covalent association with one or more heterologous substituents (e.g., a lipophilic substituent, a PEG moiety, a peptide side chain linked by a suitable organic moiety linker, etc.). A derivative wherein a heterologous substituent of significant size, such as a PEG moiety, peptide side chain, or the like, is attached to the “backbone” amino acid sequence, the derivative can be described as a “conjugate.” Thus, a derivatized fusion protein refers to a fusion protein comprising one or more of such amino acid modifications. Because fusion protein derivatives can vary significantly from their “naked” protein counterparts, they may be considered unique aspects of the invention.

In general, fusion proteins of the invention can be modified by inclusion of any suitable number of such modified amino acids. Suitability in this context generally is determined by the ability to at least substantially retain the specificity and affinity of the antibody portion(s) and target-binding portion(s) of the fusion protein if such modifications were not present.

Derivatives may be formed by producing a fusion protein comprising an antibody portion and/or target-binding portion that corresponds to a derivatized protein or by derivatizing a fusion protein comprising non-derivatized parent antibody portion and/or target-binding portion sequences.

The inclusion of one or more modified amino acids in a fusion protein of the invention may be advantageous in, for example, (a) increasing polypeptide serum half-life, (b) reducing polypeptide antigenicity, or (c) increasing polypeptide storage stability.

Amino acid (s) can be modified, for example, co-translationally or post-translationally during recombinant production (e.g., N-linked glycosylation at introduced N—X—S/T motifs during expression in mammalian cells) or modified by synthetic means. Non-limiting examples of a modified amino acid include a glycosylated amino acid, a sulfated amino acid, a prenlyated (e.g., farnesylated, geranylgeranylated) amino acid, an acetylated amino acid, an acylated amino acid, a PEGylated amino acid, a biotinylated amino acid, a carboxylated amino acid, a phosphorylated amino acid, and the like. References adequate to guide one of skill in the modification of amino acids are replete throughout the literature. Exemplary protocols are found in, e.g., Walker (1998) PROTEIN PROTOCOLS ON CD-ROM Humana Press, Towata, N.J. Typically, the modified amino acid is selected from a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent. Fusion proteins also can be chemically modified by covalent conjugation to a polymer to increase their circulating half-life, for example. Exemplary polymers and methods to attach such polymers to peptides are illustrated in, e.g., U.S. Pat. Nos. 4,766,106; 4,179,337; 4,495,285; and 4,609,546. Additional illustrative polymers include polyoxyethylated polyols and polyethylene glycol (PEG) moieties (e.g., a fusion protein can be conjugated to a PEG with a molecular weight of between about 1,000 and about 40,000, such as between about 2000 and about 20,000, e.g., about 3,000-12,000). A fusion protein also or alternatively may be conjugated to a second molecule that able to impart novel biological/pharmacological properties to the fusion protein derivative, such as a radionuclide, an enzyme, an enzyme substrate, a cofactor, a fluorescent marker, a chemiluminescent marker, a peptide tag, a magnetic particle, a toxin, or other drug. Another exemplary feature of the invention is embodied in a fusion protein that is conjugated to one or more antibody fragments, nucleic acids (oligonucleotides), nucleases, hormones, immunomodulators, chelators, boron compounds, photoactive agents, dyes, and the like.

These and other suitable agents can be coupled either directly or indirectly to fusion protein sequences of the invention. One example of indirect coupling of a second agent is coupling by a spacer moiety. These spacers, in turn, can be either insoluble or soluble (see, e.g., Diener, et al., Science, 231:148, 1986) and can be selected to enable drug release from the fusion protein at a target site and/or under particular conditions. Additional examples of therapeutic agents that can be coupled to fusion proteins include lectins and fluorescent peptides.

Methods for producing derivatives are known in the art. Methods for coupling and site-specifically conjugating PEG to a Fab′ fragment, for example, are described in Leong et al, Cytokine 16(3):106-119 (2001) and Delgado et al, Br. J. Cancer 73(2):175-182 (1996). PEG spacers typically have a MW of about 2000-4000. Such spacers can be used to conjugate derivatizing moieties or also to form fusion proteins by joining of different binding protein (typically antibody or antibody-derived, such as antibody fragment) portions. Relatively shorter spacers, for example short amino acid sequence spacers, such as a DSSP spacer, also similarly can be used to join antibody portions and/or derivatizing agents to antibody portions. Other linkers which also may be suitable are described herein and/or are known in the art (see, e.g., Kortt et al., Biomol Eng. 2001 Oct. 15; 18(3):95-108, regarding principles relevant to selection of linkers for single chain Fv antibody fragments). Antibody portions, such as Fab fragments or Fab-comprising antibody molecules also can be joined by Cys-Cys linkages, which can be facilitated by various known techniques. Joining of amino acid chains to linked moieties typically is accomplished by chemical crosslinking (such as by the affinity cross-linking methods described in U.S. Pat. No. 6,238,667). Pharmaceutical small molecules, radioactive compounds, and the like can be associated with fusion proteins in the form of chelates that attach to a molecule (e.g. biotin, avidin, streptavidin, etc.) that specifically binds an epitope tag in or attached to a fusion protein. Chelating groups are well known and include groups derived from ethylene diamine tetra-acetic acid (EDTA), diethylene triamine penta-acetic acid (DTPA), cyclohexyl 1,2-diamine tetra-acetic acid (CDTA), ethyleneglycol-O,O′-bis(−2-aminoethyl)-N,N,N′,N′-tetra-acetic acid (EGTA), N,N-bis(hydroxybenzyl)-e-thylenediamine-N,N′-diacetic acid (HBED), triethylene tetramine hexa-acetic acid (TTHA), 1,4,7,10-tetraazacyclododecane-N,N′-, N″,N′″-tetra-acetic acid (DOTA) (see, e.g., U.S. Pat. No. 5,428,156 and Lewis et al. (1994) Bioconjugate Chem. 5: 565-576), hydroxyethyldiamine triacetic acid (HEDTA),1,4,8,11-tetra-azacyclotetradecane-N,N′,N″,N′″-tetra-acetic acid (TETA), substituted DTPA, substituted EDTA, and the like.

### Pharmaceutical Compositions

In another aspect, the invention relates to compositions that comprise fusion proteins of the invention, such as pharmaceutical compositions comprising an effective amount of a fusion protein of the invention (such as a therapeutically effective amount (therapeutic dose) of such a fusion protein).

Compositions comprising a fusion protein of the invention that are intended for pharmaceutical use typically contain at least a physiologically effective amount of the fusion protein, and commonly desirably contain a therapeutically effective amount of a fusion protein, or a combination of a fusion protein and additional active/therapeutic agents (combination therapies and compositions are discussed elsewhere herein).

A “therapeutically effective amount” refers to an amount of a biologically active compound or composition that, when delivered in appropriate dosages and for appropriate periods of time to a host that typically is responsive for the compound or composition, is sufficient to achieve a desired therapeutic result in a host and/or typically able to achieve such a therapeutic result in substantially similar hosts (e.g., patients having similar characteristics as a patient to be treated). A therapeutically effective amount of a fusion protein may vary according to factors such as the disease state, age, sex, and weight of the individual, and the ability of the fusion protein to elicit a desired response in the individual. A therapeutically effective amount is also one in which any toxic or detrimental effects of the antibody or anti-body portion are outweighed by the therapeutically beneficial effects. Exemplary therapeutic effects include, e.g., (a) a reduction in the severity of a disease, disorder, or related condition in a particular subject or a population of substantial similar subject; (b) a reduction in one or more symptoms or physiological conditions associated with a disease, disorder, or condition; or (c) a prophylactic effect. A reduction of the severity of a disease can include, for example, (a) a measurable reduction in the spread of a disorder (e.g., the spread of a cancer in a patient); (b) an increase in the chance of a positive outcome in a subject (e.g., an increase of at least about 5%, 10%, 15%, 20%, 25%, or more); (c) an increased chance of survival or lifespan; and/or (d) a measurable reduction in one or more biomarkers associated with the presence of the disease state (e.g., a reduction in the amount and/or size of tumors in the context of cancer treatment; a reduction in viral load in the context of virus infection treatment; etc.). A therapeutically effective amount can be measured in the context of an individual subject or, more commonly, in the context of a population of substantial similar subjects (e.g., a number of human patients with a similar disorder enrolled in a clinical trial involving a fusion protein composition or a number of non-human mammals having a similar set of characteristics being used to test a fusion protein in the context of preclinical experiments).

A “prophylactically effective amount” refers to an amount of an active compound or composition that is effective, at dosages and for periods of time necessary, in a host typically responsive to such compound or composition, to achieve a desired prophylactic result in a host or typically able to achieve such results in substantially similar hosts. Exemplary prophylactic effects include a reduction in the likelihood of developing a disorder, a reduction in the intensity or spread of a disorder, an increase in the likelihood of survival during an imminent disorder, a delay in the onset of a disease condition, a decrease in the spread of an imminent condition as compared to in similar patients not receiving the prophylactic regimen, etc. Typically, because a prophylactic dose is used in subjects prior to or at an earlier stage of disease, the prophylactically effective amount will be less than the therapeutically effective amount for a particular fusion protein. A prophylactic effect also can include, e.g., a prevention of the onset, a delay in the time to onset, a reduction in the consequent severity of the disease as compared to a substantially similar subject not receiving fusion protein composition, etc.

A “physiologically effective” amount is an amount of an active agent that upon administration to a host that is normally responsive to such an agent results in the induction, promotion, and/or enhancement of at least one physiological effect associated with modulation of effector lymphocyte activity (e.g., increase in NK cell-associated apoptosis; increase in NK cell-associated IFNγ secretion; etc.). A therapeutically effective amount typically also is prophylactically effective and physiologically effective, but the reverse is typically not true (i.e., a physiologically effective amount may be too low of an amount or too high of an amount to be therapeutically effective).

Terms such as “treat”, “treating”, and “treatment” herein refer to the delivery of an effective amount of a therapeutically active compound or composition, such as a fusion protein composition of the invention, with the purpose of preventing any symptoms or disease state to develop or with the purpose of easing, ameliorating, or eradicating (curing) such symptoms or disease states already developed. The term “treatment” is thus meant to include prophylactic treatment. However, it will be understood that therapeutic regimens and prophylactic regimens of the invention also can be considered separate and independent aspects of this invention.

A fusion protein can be combined with one or more pharmaceutically acceptable carriers (diluents, excipients, and the like) and/or adjuvants appropriate for one or more intended routes of administration to provide compositions that are pharmaceutically acceptable. Pharmaceutically acceptable compositions comprising a therapeutic does of a fusion protein of the invention may be referred to as “pharmaceutical compositions”. Acceptability of a composition and its components is generally made in terms of toxicity, adverse side effects, undesirable immunogenicity, etc., as will be readily determinable by standard methods.

Pharmaceutically acceptable carriers generally include any and all suitable solvents, dispersion media, coatings, antibacterial and antifungal agents, isotonic and absorption delaying agents, and the like that are physiologically compatible with a fusion protein. Examples of pharmaceutically acceptable carriers include water, saline, phosphate buffered saline, dextrose, glycerol, ethanol, and the like, as well as combinations of any thereof. In many cases, it can be desirable to include isotonic agents, for example, sugars, polyalcohols such as mannitol, sorbitol, or sodium chloride in such a composition. Pharmaceutically acceptable substances such as wetting agents or minor amounts of auxiliary substances such as wetting agents or emulsifying agents, preservatives or buffers, which desirably can enhance the shelf life or effectiveness of the fusion protein, related composition, or combination. Suitability for carriers and other components of pharmaceutical compositions is determined based on the lack of significant negative impact on the desired biological properties of the fusion protein, related composition, or combination (e.g., less than a substantial impact (10% or less relative inhibition, 5% or less relative inhibition, etc.) on effector lymphocyte activating receptor and secondary target binding)

Fusion proteins of the invention may be, for example, admixed with lactose, sucrose, powders (e.g., starch powder), cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and optionally further tabletted or encapsulated for conventional administration. Alternatively, a fusion protein may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other carriers, adjuvants, and modes of administration are well known in the pharmaceutical arts. A carrier or diluent may include time delay material, such as glyceryl monostearate or glyceryl distearate alone or with a wax, or other functionally similar materials.

Fusion protein compositions, related compositions (discussed elsewhere herein e.g., compositions comprising nucleic acids encoding one of the inventive fusion proteins), and combinations according to the invention may be in a variety of suitable forms. Such forms include, for example, liquid, semi-solid and solid dosage forms, such as liquid solutions (e.g., injectable and infusible solutions), dispersions or suspensions, emulsions, microemulsions, tablets, pills, powders, liposomes, dendrimers and other nanoparticles (see, e.g., Baek et al., Methods Enzymol. 2003; 362:240-9; Nigavekar et al., Pharm Res. 2004 March; 21(3):476-83), microparticles, and suppositories. The optimal form for any fusion protein-associated composition depends on the intended mode of administration, the nature of the composition or combination, and therapeutic application or other intended use. Formulations also can include, for example, powders, pastes, ointments, jellies, waxes, oils, lipids, lipid (cationic or anionic) containing vesicles, DNA conjugates, anhydrous absorption pastes, oil-in-water and water-in-oil emulsions, emulsions, carbowax (polyethylene glycols of various molecular weights), semi-solid gels, and semi-solid mixtures containing carbowax. Any of the foregoing mixtures may be appropriate in treatments and therapies in accordance with the present invention, provided that the binding of the fusion protein to its targets is not significantly inhibited by the formulation and the formulation is physiologically compatible and tolerable with the route of administration. See also, e.g., Powell et al. “Compendium of excipients for parenteral formulations” PDA J Pharm Sci Technol. 52:238-311 (1998) and the citations therein for additional information related to excipients and carriers well known to pharmaceutical chemists.

In a particular aspect, fusion proteins are administered in liposomes (immunoliposomes). In another aspect, fusion proteins are administered in liposomes and a secondary agent, such as an antisense RNA, RNAi or siRNA for suppressing a gene in an NK cell, or toxins or drugs for the targeted killing of NK cells (additional secondary agents for combination therapies are described elsewhere herein). The production of liposomes is well known in the art. Immunoliposomes also can be targeted to particular cells by standard techniques.

Fusion protein compositions also include compositions comprising any suitable combination of a fusion protein peptide and a suitable salt therefor. Any suitable salt, such as an alkaline earth metal salt in any suitable form (e.g., a buffer salt), can be used in the stabilization of fusion proteins (preferably the amount of salt is such that oxidation and/or precipitation of the fusion protein is avoided). Suitable salts typically include sodium chloride, sodium succinate, sodium sulfate, potassium chloride, magnesium chloride, magnesium sulfate, and calcium chloride. In one aspect, an aluminum salt is used to stabilize a fusion protein in a composition of the invention, which aluminum salt also may serve as an adjuvant when such a composition is administered to a patient. Compositions comprising a base and fusion proteins also are provided. In other aspects, the invention provides a fusion protein composition that essentially lacks a tonicifying amount of any salt.

Typically, compositions in the form of injectable or infusible solutions, such as compositions similar to those used for passive immunization of humans with other antibodies, are used for delivery of fusion proteins of the invention. A typical mode for delivery of fusion protein compositions is by parenteral administration (e.g., intravenous, subcutaneous, intraperitoneal, and/or intramuscular administration). In one aspect, a fusion protein antibody is administered to a human patient by intravenous infusion or injection. In another aspect, a fusion protein antibody is administered by intramuscular or subcutaneous injection. As indicated above, intratumor administration also may be useful in certain therapeutic regimens.

Thus, fusion proteins of the invention may be formulated in, for example, solid formulations (including, e.g., granules, powders, projectile particles, or suppositories), semisolid forms (gels, creams, etc.), or in liquid forms (e.g., solutions, suspension, or emulsions).

Fusion proteins may, for example, be applied in a variety of solutions. Suitable solutions for use in accordance with the invention typically are sterile, dissolve sufficient amounts of the antibody and other components of the composition (e.g., an immunomodulatory cytokine such as GM-CSF, IL-2, and/or KGF), stable under conditions for manufacture and storage, and not harmful to the subject for the proposed application. A fusion protein may be subjected to conventional pharmaceutical operations such as sterilization and/or may contain conventional adjuvants, such as preservatives, stabilizers, wetting agents, emulsifiers, buffers etc. A composition also can be formulated as a solution, microemulsion, dispersion, powder, macroemulsion, liposome, or other ordered structure suitable to high drug concentration. Desirable fluidity properties of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin. These and other components of a pharmaceutically acceptable composition of the invention can impart advantageous properties such as improved transfer, delivery, tolerance, and the like.

A composition for pharmaceutical use also or alternatively can include various diluents, fillers, salts, buffers, detergents (e.g., a nonionic detergent, such as Tween-80), stabilizers (e.g., sugars or protein-free amino acids), preservatives, tissue fixatives, solubilizers, and/or other materials suitable for inclusion in a composition for pharmaceutical use. Examples of suitable components also are described in, e.g., Berge et al., J. Pharm. Sci., 6661),1-19 (1977); Wang and Hanson, J. Parenteral. Sci. Tech: 42, S4-S6 (1988);U.S. Pat. Nos. 6,165,779 and 6,225,289; and other documents cited herein. Such a pharmaceutical composition also can include preservatives, antioxidants, or other additives known to those of skill in the art. Additional pharmaceutically acceptable carriers are known in the art and described in, e.g., Urquhart et al., Lancet, 16, 367 (1980), Lieberman et al., Pharmaceutical Dosage Forms-Disperse Systems (2nd ed., vol. 3, 1998); Ansel et al., Pharmaceutical Dosage Forms & Drug Delivery Systems (7th ed. 2000); Martindale, The Extra Pharmacopeia (31 st edition), Remington's Pharmaceutical Sciences (16th-20th editions); The Pharmacological Basis Of Therapeutics, Goodman and Gilman, Eds. (9th ed.-1996); Wilson and Gisvolds' TEXTBOOK OF ORGANIC MEDICINAL AND PHARMACEUTICAL CHEMISTRY, Delgado and Remers, Eds. (10th ed.-1998), and U.S. Pat. Nos. 5,708,025 and 5,994,106. Principles of formulating pharmaceutically acceptable compositions also are described in, e.g., Platt, Clin. Lab Med., 7:289-99 (1987), Aulton, Pharmaceutics: The Science Of Dosage Form Design, Churchill Livingstone (New York) (1988), EXTEMPORANEOUS ORAL LIQUID DOSAGE PREPARATIONS, CSHP (1998), and “Drug Dosage,” J. Kans. Med. Soc., 70 (I), 30-32 (1969). Further additional pharmaceutically acceptable carriers particularly suitable for administration of fusion protein compositions and related compositions (e.g., compositions comprising fusion protein-encoding nucleic acids or fusion protein-encoding nucleic acid comprising vectors) are described in, for example, International Patent Application WO 98/32859.

Fusion protein compositions can be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid, and combinations of any thereof, so as to provide such a composition. Methods for the preparation of such compositions are known. See, e.g., Sustained and Controlled Release Drug Delivery Systems, J. R. Robinson, ed., Marcel Dekker, Inc., New York, 1978.

In another aspect, compositions of the invention are formulated for oral administration, for example, with an inert diluent or an assimilable edible carrier. The fusion protein (and other ingredients, if desired to be included) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

In the case of combination compositions (discussed further herein), fusion proteins can be coformulated with and/or coadministered with one or more additional therapeutic agents (e.g., an antigenic peptide and/or an immunostimulatory cytokine). Such combination therapies may require lower dosages of the fusion protein and/or the co-administered agents, thus avoiding possible toxicities or complications associated with the various monotherapies.

In another aspect, the invention provides combination compositions comprising a fusion protein of the invention and at least one second active agent, wherein the fusion protein and second active agent are present in dosages and conditions such that they produce a desired physiological, and typically a desired therapeutic, effect. The invention also provides therapeutic methods and uses comprising delivery of such a combination of agents to a subject, such as a human patient. Unless otherwise stated, all aspects described herein with respect to a particular combination composition or method may be applied to the other. Nonetheless, it should be recognized that combination methods and compositions will vary with respect to one another.

There are a number of agents that may be advantageously combined with fusion proteins of the invention and the selection of such agents will depend on the intended use of the fusion protein, the components of the fusion protein, etc.

In one context, the invention provides combination compositions and combination therapies that comprise a fusion protein of the invention that is capable of inducing or promoting a response against a cancerous or pre-cancerous condition and at least one second anti-cancer agent.

In another aspect, the invention provides combination compositions and combination therapies that comprises a fusion protein of the invention that is capable of inducing or promoting a therapeutic response against a viral infection and at least one second anti-viral agent.

In one aspect, the invention provides combination compositions and therapies wherein one or more effector lymphocyte activating compounds is/are combined with a fusion protein of the invention. For example, a fusion protein may be combined with interferon alpha (IFNα), IFNβ, interleukin (IL) 12 (IL-12), IL-18, and/or IL-2. In another aspect, a fusion protein may be combined with an agent that activates T cells, such as CTLs, e.g., IL-2 or T helper cells. The fusion protein may act on the same type of cells as the activating compounds, share some overlap in the case of multiple activating compounds, or act on a different cell type than the effector lymphocyte activating compounds with which it is combined in the method or composition.

In the case of compositions and methods used to treat cancer or as prophylaxis against cancer in the case of a patient at risk of developing a cancer (e.g., a patient in a period of remission, a patient having a detected precancerous condition, etc.), fusion proteins of the invention may be combined with one or more anti-cancer second agents. Such secondary agents can be any suitable antineoplastic therapeutic agent, such as an antineoplastic immunogenic peptide, antibody, or small molecule drug.

Drugs employed in cancer therapy may have a cytotoxic or cytostatic effect on cancer cells, or may reduce proliferation of the malignant cells. Among the texts providing guidance for cancer therapy is Cancer, PRINCIPLES AND PRACTICE OF ONCOLOGY, 4th Edition, DeVita et al., Eds. J. B. Lippincott Co., Philadelphia, Pa. (1993). An appropriate therapeutic approach is chosen according to such factors as the particular type of cancer and the general condition of the patient, as is recognized in the pertinent field.

In a particular facet, the invention provides a composition comprising a fusion protein and a suitable second anti-cancer monoclonal antibody (“mAb”) (which may include a full length mAb, a mAb fragment, or a mAb derivative). Any mAb that does not significantly interfere with the specificity, selectivity, and/or affinity of the fusion protein for its targets may be suitable, although the mAb typically also is selected for the combined effect of the mAb and the fusion protein.

In another aspect, the invention provides combination compositions and combination therapy methods involving a chemotherapeutic agent delivered to a host in association with an anti-cancer fusion protein. In a particular aspect, the fusion protein or related composition is delivered in association with a chemotherapeutic that acts on the DNA level of cancer progression. In another particular aspect, the fusion protein or related composition is delivered to a subject or comprised in a composition with an “RNA level” chemotherapeutic agent (or combination thereof), nonlimiting examples of which include Vinca alkaloid, taxanes, and topoisomerase inhibitors. A general discussion of cytotoxic agents used in chemotherapy which can provide further compositions, methods, and related principles useful in the context of chemotherapy combination compositions and administration methods is provided in, e.g., Sathe, M. et al., CANCER CHEMOTHERAPEUTIC AGENTS: HANDBOOK OF CLINICAL DATA (1978) and the second edition thereof (Preston—1982), and CANCER CHEMOTHERAPEUTIC AGENTS (ACS Professional Reference Book) (William Foye, Ed. 1995). A number of additional agents that can be useful in such contexts are set forth in Table C of U.S. Pat. No. 6,524,583.

In another exemplary aspect, the invention provides a combination composition or combination administration method, wherein a fusion protein is combined or associated with an anti-cancer nucleic acid. For example, a fusion protein can be combined with or administered in association with an anti-cancer antisense nucleic acid (e.g., augmerosen/G3139, LY900003 (ISIS 3521), ISIS 2503, OGX-011 (ISIS 112989), LE-AON/LEraf-AON (liposome encapsulated c-raf antisense oligonucleotide/ISIS-5132), MG98, and other antisense nucleic acids that target PKCα, clusterin, IGFBPs, protein kinase A, cyclin D1, or Bcl-2-see, e.g., Benimetskaya et al., Clin Prostate Cancer. 2002 June; 1 (1):20-30; Tortora et al., Ann N Y Acad Sci. 2003 December; 1002:236-43; Gleave et al., Ann N Y Acad. Sci. 2003 December; 1002:95-104.; Lahn et al., Ann N Y Acad. Sci. 2003 December; 1002:263-70; Kim et al., Int J. Oncol. 2004 January; 24(1):5-17; Stahel et al., Lung Cancer. 2003 August; 41 Suppl 1:S81-8; Stephens et al., Curr Opin Mol. Ther. 2003 April; 5(2):118-22; Cho-Chung, Arch Pharm Res. 2003 March; 26(3):183-91; and Chen, Methods Mol. Med. 2003; 75:621-36)).

In another aspect, a fusion protein is administered in association with or combined in a composition with an anti-cancer inhibitory RNA molecule (see, e.g., Lin et al., Curr Cancer Drug Targets. 2001 November; 1 (3):241-7, Erratum in: Curr Cancer Drug Targets. 2003 June; 3(3):237; Lima et al., Cancer Gene Ther. 2004 May; 11 (5):309-16; Grzmil et al., Int J Oncol. 2004 January; 24(1):97-105; Collis et al., Int J Radiat Oncol Biol Phys. 2003 Oct. 1; 57(2 Suppl):S144; Yang et al., Oncogene. 2003 Aug. 28; 22(36):5694-701; and Zhang et al., Biochem Biophys Res Commun. 2003 Apr. 18; 303(4):1169-78)).

In another facet, the invention provides combination compositions and combination administration methods where a fusion protein is combined with an anti-cancer nucleozyme, such as a ribozyme, an example of which is angiozyme (Ribozyme Pharmaceuticals) (see e.g., Pennati et al., Oncogene. 2004 Jan. 15; 23(2):386-94; Tong et al., Clin Lung Cancer. 2001 February; 2(3):220-6; Kijima et al., Int J. Oncol. 2004 March; 24(3):559-64; Tong et al., Chin Med J (Engl). 2003 October; 116(10):1515-8; and Orlandi et al., Prostate. 2003 Feb. 1; 54(2):133-43). In yet another aspect, a fusion protein is combined with an immunostimulatory nucleic acid (see, e.g., Krieg, Trends in Microbiol 7: 64-65 (1999); Wooldridge et al., Curr Opin Oncol. 2003 November; 15(6):440-5; Jahrsdorfer et al., Semin Oncol. 2003 August; 30(4):476-82; Jahrsdorfer et al., Curr Opin Investig Drugs. 2003 June; 4(6):686-90; and Carpentier et al., Front Biosci. 2003 Jan. 1; 8:e115-27).

In another aspect, the invention provides combination compositions and methods, wherein a fusion protein is combined with or administered in association with a tumor suppressor-encoding nucleic acid. In one exemplary aspect, the tumor suppressor is a p53 tumor suppressor gene (see, e.g., Roth et al., Oncology (Huntingt). 1999 October; 13(10 Suppl 5):148-54) and Nielsen et al., Cancer Gene Ther. 1998 January-February; 5(1):52-63). Additional tumor suppressor targets include, for example, BRCA1, RB1, BRCA2, DPC4 (Smad4), MSH2, MLH1, and DCC.

In another aspect, the invention provides combination compositions and combination administration methods wherein a fusion protein is combined or coadministered with an oncolytic virus. Examples of such viruses include oncolytic adenoviruses and herpes viruses, which may or may not be modified herpes viruses (examples of such viruses and related principles thereto are described in, e.g., Teshigahara et al., J Surg Oncol. 2004 January; 85(1):42-7; Stiles et al., Surgery. 2003 August; 134(2):357-64; Zwiebel et al., Semin Oncol. 2001 August; 28(4):336-43; Varghese et al., Cancer Gene Ther. 2002 December; 9(12):967-78; and Wildner et al., Cancer Res. 1999 Jan. 15; 59(2):410-3).

Viruses, viral proteins, and the like also can be used in combination compositions and combination administration methods. Replication-deficient viruses, that generally are capable of one or only a few rounds of replication in vivo, and that are targeted to tumor cells, can, for example, be useful components of such compositions and methods. Such viral agents can comprise or be associated with nucleic acids encoding immunostimulants, such as GM-CSF and/or IL-2. Both naturally oncolytic and such recombinant oncolytic viruses (e.g., HSV-1 viruses; reoviruses; replication-deficient and replication-sensitive adenovirus; etc.) can be useful components of such methods and compositions (see, e.g., Varghese et al., Cancer Gene Ther. 2002 December; 9(12):967-78; Zwiebel et al., Semin Oncol. 2001 August; 28(4):336-43; Sunarmura et al., Pancreas. 2004 April; 28(3):326-9; Shah et al., J Neurooncol. 2003 December; 65(3):203-26; and Yamanaka, Int J Oncol. 2004 April; 24(4):919-23).

As an additional feature, the invention provides combination administration methods and combination compositions wherein a fusion protein is delivered in association with an anti-cancer immunogen, such as a cancer antigen/tumor-associated antigen (e.g., an epithelial cell adhesion molecule (Ep-CAM/TACSTD1), mucin 1 (MUC1), carcinoembryonic antigen (CEA), tumor-associated glycoprotein 72 (TAG-72), gp100, Melan-A, MART-1, KDR, RCAS1, MDA7, cancer-associated viral vaccines (e.g., human papillomavirus vaccines), tumor-derived heat shock proteins, and the like) (see also, e.g., Acres et al., Curr Opin Mol Ther 2004 Feb., 6:40-7; Taylor-Papadimitriou et al., Biochim Biophys Acta. 1999 Oct. 8; 1455(2-3):301-13; Emens et al., Cancer Biol Ther. 2003 July-August; 2(4 Suppl 1):S161-8; and Ohshima et al., Int J Cancer. 2001 Jul. 1; 93(1):91-6).

Compositions and combination administration methods of the invention also include the inclusion or coadministration of nucleic acid vaccines, such as naked DNA vaccines encoding such cancer antigens/tumor-associated antigens (see, e.g., U.S. Pat. Nos. 5,589,466, 5,593,972, 5,703,057, 5,879,687, 6,235,523, and 6,387,888). In another aspect, the combination administration method and/or combination composition comprises an autologous vaccine composition. In a further aspect, the combination composition and/or combination administration method comprises a whole cell vaccine or cytokine-expressing cell (e.g., a recombinant IL-2 expressing fibroblast, recombinant cytokine-expressing dendritic cell, and the like) (see, e.g., Kowalczyk et al., Acta Biochim Pol. 2003; 50(3):613-24; Reilly et al., Methods Mol. Med. 2002; 69:233-57; Ferlazzo et al., J. Exp. Med., 153):343-351 (2002); and Tirapu et al., Curr Gene Ther. 2002 February; 2(1):79-89). Another example of a therapeutic autologous cell method that can be useful in combination methods of this invention is the MyVax® Personalized Immunotherapy method (previously referred to as GTOP-99) (available through Genitope Corporation—Redwood City, Calif., USA) (see U.S. Pat. Nos. 5,972,334 and 5,776,746). In a different aspect, the inventive methods can be practiced by methods that also or alternatively comprise co-delivery of one or more types of NK cells (e.g., a population of CD56dimCD16+ NK cells), which can be genetically modified and/or modified by various contact with substances (e.g., one or more activating factors) prior to delivery.

In another aspect, the invention provides a combination composition or combination administration method comprising a fusion protein and an anti-cancer cytokine, chemokine, or combination thereof. Any suitable anti-cancer cytokine and/or chemokine can be used with and/or combined with fusion proteins in the methods and compositions of the invention. Suitable chemokines and cytokines result in a detectably greater and/or more comprehensive immune response to cancer cells or related tissues (e.g., tumors) in vivo and do not substantially impede the binding of the fusion protein(s) in the composition/method.

In another aspect, the invention provides combination compositions and combination administration methods comprising a fusion protein and an adjuvant, typically in further combination with an anti-cancer immunogenic peptide. Non-limiting examples of suitable adjuvants are QS21, SRL-172, histamine dihydrochloride, thymocartin, Tio-TEPA, monophosphoryl-lipid A/micobacteria compositions, alum, incomplete Freund's Adjuvant, Montanide ISA, Ribi Adjuvant System, TiterMax adjuvant, syntex adjuvant formulations, immune-stimulating complexes (ISCOMs), GerbuR adjuvant, CpG oligodeoxynucleotides, lipopolysaccharide, and polyinosinic:polycytidylic acid.

In yet another aspect, the invention provides combination compositions and combination administration methods comprising a telomerase inhibitor, telomerase vaccine, or combination thereof in addition to at least one fusion protein or related molecule. Examples of such compositions and related techniques and principles are described in, e.g., U.S. Pat. Nos. 6,440,735 and 6,713,055.

In a further aspect, combination compositions and/or combination administration methods of the invention comprise administration of an immunomodulatory compound or modulator thereof (e.g., an anti-inhibitory immunomodulatory antibody). Examples of such compounds include B7 molecules. Another example of such a molecule is an inhibitor of a negative T cell regulator, such as an antibody against CTLA4 or against another negative immune cell regulator, such as BTLA and PD-1. In another aspect, delivery of such inhibitory molecules may be desired, for example in the treatment of autoimmune diseases or other immune system related disorders. In a further exemplary aspect, an inhibitor of CD4, such as an anti-CD4 antibody can be delivered in association with practice of inventive methods provided here.

In another facet, a combination composition or combination administration method comprises one or more immunosuppressive/immunomodulatory agents, such as a T lymphocyte homing modulator; a calcineurin inhibitor; or a TOR-inhibitor.

In another aspect, the invention provides combination compositions and combination administration methods that involve at least one fusion protein and one or more cell cycle control/apoptosis regulators (or cell cycle/apoptosis “regulating agents”). A cell cycle control/apoptosis regulator can include, for example, one or more molecules that target and modulate cell cycle control/apoptosis regulators.

In yet another aspect, the invention provides combination compositions and combination administration methods that comprise one or more growth factor inhibitors. A number of mAbs against growth factors and growth factor receptors are known that can be useful in promoting the treatment of cancer. For example, antibodies against the extracellular ligand binding domain of epidermal growth factor receptor (EGF-R) proteins that are abnormally activated in epithelial tumors can be useful in the treatment of aggressive epithelial cell-derived tumors. Antibodies against low molecular weight molecules that inhibit the tyrosine kinase domains of such receptors also can be useful in combination compositions or combination administration methods.

Other features of the invention include combination compositions and combination administration methods that comprise an inhibitor of angiogenesis, neovascularization, and/or other vascularization delivered in association with one or more fusion proteins.

In yet another aspect, the invention provides combination compositions and combination administration methods wherein at least one fusion protein is combined with a hormonal regulating agent, such as an anti-androgen and/or anti-estrogen therapy agent or regimen.

Combination compositions and combination administration methods also or alternatively can involve “whole cell” and “adoptive” immunotherapy methods. For example, such methods can comprise infusion or re-infusion of immune system cells. Cell lysates also may be useful in such methods and compositions. Cellular “vaccines” in clinical trials that may be useful in such aspects include Canvaxin™, APC-8015 (Dendreon), HSPPC-96 (Antigenics), and Melacine® cell lysates. Antigens shed from cancer cells, and mixtures thereof (see, e.g., Bystryn et al., Clinical Cancer Research Vol. 7, 1882-1887, July 2001), optionally admixed with adjuvants such as alum, also can be advantageous components in such methods and methods. U.S. Pat. No. 6,699,483 provides another example of a whole cell anti-cancer therapy. Additional examples of such whole cell immunotherapies that can be usefully combined in fusion protein-related compositions and methods are described elsewhere herein.

In another aspect, the invention provides combination compositions and combination administration methods comprising one or more immune system inhibitors Intracellular signaling inhibitors. Examples of such compounds include tyrosine kinase inhibitors, modulators of the ras signaling pathway, and regulators of protein trafficking. Other examples include serine/threonine kinase inhibitors, protein-tyrosine phosphatases inhibitors, dual-specificity phosphatases inhibitors, and serine/threonine phosphatases inhibitors.

Combination compositions and combination administration methods also or alternatively can include anti-anergic agents (e.g., small molecule compounds, proteins, glycoproteins, or antibodies that break tolerance to tumor and cancer antigens).

In yet another aspect, a fusion protein can be delivered to a patient in combination with the application of an internal vaccination method. Internal vaccination refers to induced tumor or cancer cell death, such as drug-induced or radiation-induced cell death of tumor cells, in a patient, that typically leads to elicitation of an immune response directed towards (i) the tumor cells as a whole or (ii) parts of the tumor cells including (a) secreted proteins, glycoproteins or other products, (b) membrane-associated proteins or glycoproteins or other components associated with or inserted in membranes, and/or (c) intracellular proteins or other intracellular components. An internal vaccination-induced immune response may be humoral (i.e. antibody—complement-mediated) or cell-mediated (e.g., the development and/or increase of endogenous cytotoxic T lymphocytes that recognize the internally killed tumor cells or parts thereof). In addition to radiotherapy, non-limiting examples of drugs and agents that can be used to induce said tumor cell-death induction and internal vaccination methods include conventional chemotherapeutic agents, cell-cycle inhibitors, anti-angiogenesis drugs, monoclonal antibodies, apoptosis-inducing agents, and signal transduction inhibitors.

Additional agents that can be comprised in the combination compositions and/or combination administration methods of the invention include fluoropyrimidiner carbamates; non-polyglutamatable thymidylate synthase inhibitors; nucleoside analogs; antifolates; topoisomerase inhibitors; polyamine analogs; mTOR inhibitors; alkylating agents; lectin inhibitors; vitamin D analogs; carbohydrate processing inhibitors; antimetabolism folate antagonists; thumidylate synthase inhibitors; antimetabolites (e.g., raltitrexed); ribonuclease reductase inhibitors; dioxolate nucleoside analogs; thimylate syntase inhibitors; gonadotropin-releasing hormone (GRNH) peptides; human chorionic gonadotropin; and chemically modified tetracyclines.

Useful prophylactic and therapeutic regimens of the invention also or alternatively can be combined with anti-cancer directed photodynamic therapy (e.g., anti-cancer laser therapy—which optionally can be practiced with the use of photosensitizing agent, see, e.g., Zhang et al., J Control Release. 2003 Dec. 5; 93(2):141-50); anti-cancer sound-wave and shock-wave therapies (see, e.g., Kambe et al., Hum Cell. 1997 March; 10(1):87-94); anti-cancer thermotherapy (see, e.g., U.S. Pat. No. 6,690,976), and/or anti-cancer nutraceutical therapy (see, e.g., Roudebush et al., Vet Clin North Am Small Anim Pract. 2004 January; 34(1):249-69, viii and Rafi, Nutrition. 2004 January; 20(1):78-82).

Further teachings relevant to cancer combination therapies and compositions that may be applied in connection with fusion proteins of the invention are provided in, e.g., Berczi et al., “Combination Immunotherapy of Cancer” in NEUROIMMUNE BIOLOGY Volume 1: New foundation of Biology, Berczi I, Gorczynski R, Editors, Elsevier, 2001; pp. 417-432.

The invention also provides kits comprising one or more fusion proteins or related agents (e.g., fusion protein-encoding nucleic acids, vectors comprising the same, and cells comprising the same). A kit may include, in addition to the fusion protein, diagnostic or therapeutic agents. A kit may also include instructions for use in a diagnostic or therapeutic method. Such instructions can be, for example, provided on a device included in the kit. Advantageously, such a kit includes a fusion protein and a diagnostic agent that can be used in the diagnostic methods described below. In another preferred embodiment, the kit includes a fusion protein, related compound, or combination composition in a highly stable form (such as in a lyophilized form) in combination with pharmaceutically acceptable carrier(s) that can be mixed with the highly stable composition to form an injectable composition for near term administration. Such kits also can be provided with one or more other non-active pharmaceutical composition ingredients, such as a stabilizer, a preservative, a solubilizer, a solvent, a solute, a flavorant, a coloring agent, etc. For diagnostic and certain therapeutic applications, the invention provides a composition comprising one or more fusion proteins linked to a solid support, such as of the type commonly used to support antibodies (e.g., an affinity chromatography column bead or other support; a diagnostic protein microarray “chip”; a BIACORE SPR device chip; etc.).

### Therapeutic Methods

In another aspect, the invention provides therapeutic methods involving fusion proteins, fusion protein compositions, and/or related compositions. Fusion proteins of the invention can be useful in a variety of therapeutic and prophylactic regimens including, for example, the treatment of cancer, viral infections, and immune system-related disorders.

In one exemplary aspect, the invention provides a method of reducing cancer progression in a mammalian host, such as a human patient, having a detectable level of cancer cells or pre-cancer cells comprising administering a fusion protein, a fusion protein composition, or a related composition (e.g., a nucleic acid encoding a fusion protein), in an amount sufficient to detectably reduce the progression of the cancer in the host.

In a particular aspect, the target-binding portion of a fusion protein of the invention comprises a ligand-binding segment of the NKG2D-receptor. NKG2D binds to multiple ligands, including members of the MIC-A, MIC-B and ULBP families. These all are stress-inducible ligands whose expression is induced in several types of tumors. For instance, in most normal tissues, MIC-A is not expressed, but MIC-A is upregulated in various types of tumors, including epithelial breast, lung and colorectal cancers, leukemias, and gliomas (Groh et al PNAS1999; 96:6879-84).

Cancer cells are cells that divide and reproduce abnormally with uncontrolled growth (e.g., by exceeding the “Hayflick limit” of normal cell growth (as described in, e.g., Hayflick, Exp. Cell Res., 37, 614 (1965)). “Cancers” generally consist of single or several clones of cells that are capable of partially independent growth in a host (e.g., a benign tumor) or fully independent growth in a host (malignant cancer). Cancer cells arise from host cells via neoplastic transformation (“carcinogenesis”).

Terms such as “preneoplastic,” “premalignant,” and “precancerous” with respect to the description of cells and/or tissues herein refer to cells or tissues having a genetic and/or phenotypic profile that signifies a significant potential of becoming cancerous. Usually such cells can be characterized by one or more differences from their nearest normeoplastic counterparts that signal the onset of cancer progression or significant risk for the start of cancer progression. Such precancerous changes, if detectable, can usually be treated with excellent results. In general, a precancerous state will be associated with the incidence of neoplasm(s) or preneoplastic lesion(s). Examples of known and likely preneoplastic tissues include ductal carcinoma in situ (DCIS) growths in breast cancer, cervical intra-epithelial neoplasia (CIN) in cervical cancer, adenomatous polyps of colon in colorectal cancers, atypical adenomatous hyperplasia in lung cancers, and actinic keratosis (AK) in skin cancers. Pre-neoplastic phenotypes and genotypes for various cancers, and methods for assessing the existence of a preneoplastic state in cells, have been characterized. See, e.g., Medina, J Mammary Gland Biol Neoplasia. 2000 October; 5(4):393-407; Krishnamurthy et al., Adv Anat Pathol. 2002 May; 9(3):185-97; Ponten, Eur J Cancer. 2001 October; 37 Suppl 8:S97-113; Niklinski et al., Eur J Cancer Prev. 2001 June; 10(3):213-26; Walch et al., Pathobiology. 2000 January-February; 68(1):9-17; and Busch, Cancer Surv. 1998; 32:149-79. Gene expression profiles can increasingly be used to differentiate between normal, precancerous, and cancer cells. For example, familial adenomatous polyposis genes prompt close surveillance for colon cancer; mutated p53 tumor-suppressor gene flags cells that are likely to develop into aggressive cancers; osteopontin expression levels are elevated in premalignant cells, and increased telomerase activity also can be a marker of a precancerous condition (e.g., in cancers of the bladder and lung). In one aspect, the invention relates to the treatment of precancerous cells. In another aspect, the invention relates to the preparation of medicaments for treatment of precancerous cells.

“Cancer progression” refers to any event or combination of events that promote, or which are indicative of, the transition of a normal, non-neoplastic cell to a cancerous, neoplastic cell, the migration of such neoplastic cells, and the formation and growth of tumors therefrom (which latter aspect can be referred to as tumor progression). Examples of such events include phenotypic cellular changes associated with the transformation of a normal, non-neoplastic cell to a recognized pre-neoplastic phenotype, and cellular phenotypic changes that indicate transformation of a pre-neoplastic cell to a neoplastic cell. Typical and specific stages of cancer include cell crisis, immortalization and/or normal apoptotic failure, proliferation of immortalized and/or pre-neoplastic cells, transformation (i.e., changes which allow the immortalized cell to exhibit anchorage-independent, serum-independent and/or growth-factor independent, or contact inhibition-independent growth, or that are associated with cancer-indicative shape changes, aneuploidy, and focus formation), proliferation of transformed cells, development of metastatic potential, migration and metastasis (e.g., the disassociation of the cell from a location and relocation to another site), new colony formation, tumor formation, tumor growth, and neotumorogenesis (formation of new tumors at a location distinguishable and not in contact with the source of the transformed cell(s)). Carcinogenesis, the initial stage of cancer progression, is typically associated with the activation of genes that regulate cell growth via bypassing the host cell's regulatory controls (e.g., bypassing or overcoming a host cell's normally active apoptotic signaling pathway(s)) and the reduced expression of tumor-suppressor genes. Neoplastic conversion is the transformation of a preneoplastic cell into one that expresses a neoplastic phenotype. Cancer progression often is also or alternatively (and more generally) described by the general stages of initiation, promotion, and progression. In tumor-forming cancers, for example, cancer progression often is described in terms of tumor initiation, tumor promotion, malignant conversion, and tumor progression (see, e.g., CANCER MEDICINE, 5th Edition (2000) B.C. Decker Inc., Hamilton, Ontario, Canada (Blast et al. eds.)). In another and later stage of cancer progression, immunogenic tumors typically escape immune-surveillance of the host enabling their growth. Additional mid to late stage aspects of cancer progression include evasion of apoptosis by the cancer cell, achieving limitless replication potential, achieving self-sufficiency in growth factor expression, achieving abnormal insensitivity to anti-growth signals; achieving sustained angiogenesis, and metastasis. Metastasis refers to the stage of cancer progression associated with the spread of cancer cells from one site in a medium to another, such as in the tissue(s) of a patient. Metastasis also typically is involved with a number of distinct physiological events, which include the escape of cancer cells from an initial site via lymphatic channels or protease activity; the survival of cancer cells in circulation; arrest in secondary site(s); extravasation into surrounding tissue; initiation and maintenance of growth, and vascularization of metastatic tumor(s).

In general, fusion proteins of the invention can be used to treat patients suffering from any stage of cancer progression (and to prepare medicaments for reduction, delay, or other treatment of cancer progression), however the treatment of patients in the later stages of cancer progression with fusion proteins and compositions of the invention is a particularly advantageous aspect of the invention.

Cancer progression (and thus the reduction thereof) can be detected by any variety of suitable methods. Methods for detecting cancers and cancer progression include (a) clinical examination (symptoms can include swelling, palpable lumps, enlarged lymph nodes, bleeding, visible skin lesions, and weight loss); (b) imaging (X-ray techniques, mammography, colonoscopy, computed tomography (CT and/or CAT) scanning, magnetic resonance imaging (MRI), etc.); (c) immunodiagnostic assays (e.g., detection of CEA, AFP, CA125, etc.); (d) antibody-mediated radioimaging; and (e) analyzing cellular/tissue immunohistochemistry. Other examples of suitable techniques for assessing a cancerous state and cancer progression include PCR and RT-PCR (e.g., of cancer cell associated genes or “markers”), biopsy, electron microscopy, positron emission tomography (PET), computed tomography, immunoscintigraphy and other scintegraphic techniques, magnetic resonance imaging (MRI), karyotyping and other chromosomal analysis, immunoassay/immunocytochemical detection techniques (e.g., differential antibody recognition), histological and/or histopathologic assays (e.g., of cell membrane changes), cell kinetic studies and cell cycle analysis, ultrasound or other sonographic detection techniques, radiological detection techniques, flow cytometry, endoscopic visualization techniques, and physical examination techniques.

In general, delivering fusion proteins of the invention to a subject (either by direct administration or expression from a nucleic acid) and practicing the other methods of the invention can be used to reduce, treat, prevent, or otherwise ameliorate any suitable aspect of cancer progression in a subject.

A reduction of cancer progression can include, e.g., any detectable decrease in (1) the rate of normal cells transforming to neoplastic cells (or any aspect thereof), (2) the rate of proliferation of pre-neoplastic or neoplastic cells, (3) the number of cells exhibiting a pre-neoplastic and/or neoplastic phenotype, (4) the physical area of a cell media (e.g., a cell culture, tissue, or organ (e.g., an organ in a mammalian host)) comprising pre-neoplastic and/or neoplastic cells, (5) the probability that normal cells and/or preneoplastic cells will transform to neoplastic cells, (6) the probability that cancer cells will progress to the next aspect of cancer progression (e.g., a reduction in metastatic potential), or (7) any combination thereof. Such changes can be detected using any of the above-described techniques or suitable counterparts thereof known in the art, which typically are applied at a suitable time prior to the administration of a therapeutic regimen so as to assess its effectiveness. Times and conditions for assaying whether a reduction in cancer potential has occurred will depend on several factors including the type of cancer, type and amount of fusion protein, related composition, or combination composition being delivered to the host. The accomplishment of these goals by delivery of fusion proteins of the invention is another advantageous facet of this invention.

Other methods useful for diagnosing cancer progression include tumor grading and staging methods, such as the American Joint Commission on Cancer grading system, the National Program of Cancer Registries “General Staging” method (also known as Summary Staging, California Staging, and SEER Staging), and/or commonly used specialized grading systems (e.g., a high Gleason tumor grade score is indicative of an aggressive cancer in the context of prostate cancer; a TNM (Tumor, Nodes, Metastasis) Staging System often is useful in the context of colorectal cancer, and the Scarff-Bloom-Richardson system often is used in the context of breast cancer assessments). Further methods for identifying cancer and/or diagnosing cancer progression include cancer gene-related DNA methylation (see, e.g., Carmen et al., J. Natl. Cancer Inst., 93(22) (2001)), DNA cytometry, mitosis assays (as to frequency, normalcy, or both), pleomorphism evaluations, the presence of autocrine stimulatory loop activity, tubule formation measurements, keritinization assays, intercellular bridge formation assays, epithelial pearl detection, aberrant hormone receptor expression or form production assays (e.g., Her2 overexpression assays), and other cancer-associated gene expression assays (e.g., PRL-3 protein tyrosine phosphatase gene expression assays). The reduction of cancer progression, as measured by any of the foregoing assays, by delivery of fusion proteins comprising antibody portions and target-binding portions, is another advantageous facet of the invention.

The methods of the invention can be used to reduce the cancer progression of any suitable type of cancer. Forms of cancer that may be treated by the delivery or administration of fusion proteins, fusion protein compositions, and combination compositions provided by the invention include squamous cell carcinoma, leukemia, acute lymphocytic leukemia, acute lymphoblastic leukemia, B-cell lymphoma, T-cell lymphoma, Hodgkins lymphoma, non-Hodgkins lymphoma, hairy cell lymphoma, Burketts lymphoma, acute or chronic myelogenous leukemias, promyelocytic leukemia, fibrosarcoma, rhabdomyoscarcoma; melanoma, seminoma, teratocarcinoma, neuroblastoma, glioma, astrocytoma, neuroblastoma, glioma, schwannomas; fibrosarcoma, rhabdomyoscaroma, osteosarcoma, melanoma, xeroderma pigmentosum, keratoacanthoma, seminoma, thyroid follicular cancer, and teratocarcinoma. fusion proteins also can be useful in the treatment of other carcinomas of the bladder, breast, colon, kidney, liver, lung, ovary, prostate, pancreas, stomach, cervix, thyroid or skin. Fusion proteins also may be useful in treatment of other hematopoietic tumors of lymphoid lineage, other hematopoietic tumors of myeloid lineage, other tumors of mesenchymal origin, other tumors of the central or peripheral nervous system, and/or other tumors of mesenchymal origin. Advantageously, the methods of the invention also may be useful in reducing cancer progression in prostate cancer cells, melanoma cells (e.g., cutaneous melanoma cells, ocular melanoma cells, and/or lymph node-associated melanoma cells), breast cancer cells, colon cancer cells, and lung cancer cells. The methods of the invention can be used to reduce cancer progression in both tumorigenic and non-tumorigenic cancers (e.g., non-tumor-forming hematopoietic cancers). The methods of the invention are particularly useful in the treatment of epithelial cancers (e.g., carcinomas) and/or colorectal cancers, breast cancers, lung cancers, vaginal cancers, cervical cancers, and/or squamous cell carcinomas (e.g., of the head and neck). Additional potential targets include sarcomas and lymphomas. Additional advantageous targets include solid tumors and/or disseminated tumors (e.g., myeloid and lymphoid tumors, which can be acute or chronic).

In another exemplary aspect, the invention provides a method of increasing the ratio of quiescent to invasive neoplastic cells in a mammalian host comprising administering a therapeutically effective amount of a fusion protein (e.g., a fusion protein antibody), related composition, or combination composition of the invention so as to increase the ratio of quiescent to invasive cells in the host.

In an even further aspect, the invention provides a method for reducing the risk of developing a cancerous condition, reducing the time to onset of a cancerous condition, reducing the severity of a cancer diagnosed in the early stages, and/or reducing the affected area of a cancer upon development thereof in a mammalian host, comprising administering to a host a prophylactically effective amount of a fusion protein, related compound, or combination composition of the invention so as to achieve the desired physiological effect(s).

In another aspect, the invention provides methods for inhibiting tumor growth and/or metastasis in an individual in need thereof, comprising contacting the tumor with an amount of a fusion protein, related composition, or combination composition of the invention, so as to inhibit tumor growth and/or metastasis. Target tumors can include, but are not limited to, carcinomas. Such carcinomas include, but are not limited to squamous cell carcinomas (including but not limited to squamous cell carcinoma of skin, cervix, and vulva), gastric carcinomas, colon adenocarcinomas, colorectal carcinomas, and cervical carcinomas. Other carcinomas that can be treated by inventive methods described herein include ductal mammary carcinomas. Other common cancers that can be treated by inventive methods described herein include malignant melanomas.

Inhibiting tumor growth generally means causing a reduction in the amount of tumor growth that would occur in the absence of treatment and/or substantially complete cessation of detectable tumor growth, and includes decreases in tumor size and/or decrease in the rate of tumor growth. Inhibiting metastases means to reduce the amount of tumor metastasis that would occur in the absence of treatment, and includes a relative decrease in the number and/or size of metastases.

In still a different aspect, the inventive methods can provide means for eliciting, promoting, and/or enhancing an anti-tumor effect by slowing the growth, spread, or growth and spread of the front of a tumor into surrounding tissues, or the expected growth, spread, or growth and spread of a tumor. Tumor cell growth inhibition can be measured by any suitable standard and technique using, e.g., other methods described herein and/or inhibition assays such as are described in WO 89/06692.

An additional aspect of the invention is to provide a method for inhibiting or slowing the growth and/or spreading of a tumor into surrounding tissue by delivering to a patient in need thereof fusion protein antibody or other effective fusion protein, related compound, or combination composition.

In a further aspect, the invention provides a method of increasing the likelihood of survival over a relevant period in a human patient diagnosed with cancer. For example, the invention provides a method of increasing the likelihood of survival about six months, about nine months, about one year, about three years, or longer after treatment with a fusion protein composition of the invention, as compared to not receiving treatment with the fusion protein composition (survival rates can be determined by, e.g., studies on a population of similar patients, such as in the context of a clinical trial).

In another aspect, the invention provides a method for improving the quality of life of a cancer patient comprising administering to the patient a composition of the invention in an amount effective to improve the quality of life thereof. Methods for assessing patient quality of life in cancer treatment are well known in the art (see, e.g., Movass and Scott, Hematol Oncol Clin North Am. 2004 February; 18(1):161-86; Dunn et al., Aust N Z J Public Health. 2003; 27(1):41-53; Morton and Izzard, World J. Surg. 2003 July; 27(7):884-9; Okamato et al., Breast Cancer. 2003; 10(3):204-13; Conroy et al., Expert Rev Anticancer Ther. 2003 August; 3(4):493-504; List et al., Cancer Treat Res. 2003; 114:331-51; and Shimozuma et al., Breast Cancer. 2002; 9(3):196-202).

In a further aspect, inventive methods described herein can be applied to significantly reduce the number of cancer cells in a vertebrate host, such that, for example, the total number and/or size of tumors are reduced. Such methods can be applied to treat any suitable type of tumor including chemoresistant tumors, solid tumors, and/or metastasized tumors. In a related sense, the invention provides a method for killing preneoplastic and/or neoplastic cells in a vertebrate, such as a human cancer patient.

In another aspect, the invention provides a method of treating a viral infection in a patient or host that comprises administering or otherwise delivering a therapeutically effective amount of a fusion protein, a fusion protein composition, or combination composition so as to reduce the severity, spread, symptoms, or duration of such infection. Any virus normally associated with the activity of effector lymphocytes, such as NK cells, can be treated by the method. For example, such a method can be used to treat infection by one or more viruses selected from hepatitis type A, hepatitis type B, hepatitis type C, influenza, varicella, adenovirus, herpes simplex type I (HSV-1), herpes simplex type 2 (HSV-2), rinderpest, rhinovirus, echovirus, rotavirus, respiratory syncytial virus, papilloma virus, papilloma virus, cytomegalovirus (CMV—e.g., HCMV), echinovirus, arbovirus, huntavirus, coxsackie virus, mumps virus, measles virus, rubella virus, polio virus, and/or human immunodeficiency virus type I or type 2 (HIV-1, HIV-2). The practice of such methods may result in a reduction in the titer of virus (viral load), reduction of the number of virally infected cells, etc. In a particular aspect, these methods are practiced in immunocompromised/immunosuppressed individuals. In another aspect, these methods are practiced in patients at relatively higher risk of immunosuppression or having a relatively defective immune system, such as in young children (e.g., children of about 10 years or less, about 8 years or less, about 6 years or less, about 5 years or less, about 4 years or less, about 3 years or less, about 2 years or less in age, about 1-18 months, about 1-12 months, about 1-9 months, about 1-6 months, or less than about 3 months in age) or the elderly (e.g., patients of about 65 years or more, about 70 years or more, about 75 years or more, about 80 years or more, about 85 years or more in age, etc.). In other inventive methods (e.g., the treatment of cancer) the inventive method can be similarly limited to population groups wherein general effectiveness is expected to be improved. As discussed elsewhere herein, in certain contexts, specificity of a fusion protein can lead to definition of significant population groups, such as Caucasians that generally possess a type A haplotype, for example wherein the fusion protein is cross-reactive for KIRs relevant to such a population.

Fusion proteins can be administered with or in association with anti-viral agents, such as protease inhibitor (e.g. acyclovir) in the context of HIV treatment or an anti-viral anti-body (e.g., an anti-gp41 antibody in the context of HIV treatment; an anti-CD4 antibody in the context of the treatment of CMV, etc.). Numerous types of anti-viral agents for the above-described viruses are known with respect to each type of target virus.

In another aspect, the invention provides a method of treating a disease caused by bacteria, protozoa, molds, or fungi, comprising administering or otherwise delivering a therapeutically effective amount of a fusion protein or fusion protein composition to a patient or host in need thereof for reducing the severity, spread, symptoms, or duration of such an infection therein.

In one exemplary aspect, such a method can be used to treat a patient suffering from an infectious disease caused by a bacteria, protozoa, or parasite selected from Staphylococcus, S. pyogenes, Enterococcl, Bacillus anthracis, Lactobacillus, Listeria, Corynebacterium diphtheriae, G. vaginalis; Nocardia; Streptomyces; Thermoactinomyces vulgaris; Treponerna; Camplyobacter, Raeruginosa; Legionella; N. gonorrhoeae; N. meningitides; F. meningosepticum; F. odoraturn; Brucella; B. pertussis; B. bronchiseptica; E. coli; Klebsiella; Enterobacter; S. marcescens; S. liquefaciens; Edwardsiella; P. mirabilis; P. vulgaris; Streptobacillus; R. fickettsfi; C. psittaci; C. trachornatis; M. tuberculosis, M. intracellulare, M. folluiturn, M. laprae, M. avium, M. bovis, M. africanum, M. kansasii, M. intracellulare; M. lepraernurium; Nocardia, other Streptococcus, other Bacillus, other Gardnerella, other Pseudomonas, other Neisseria, other Flavobacterium, other Bordetella, other Escherichia, other Serratia, other Proteus, other Rickettsiaceae, other Chlamydia, other Mycobacterium, leishmania, kokzidioa, trypanosome, chlamydia or rickettsia.

In another aspect, fusion proteins are administered or otherwise delivered to a patient in association with transplantation (e.g., the grafting or insertion of cells, tissue(s) or organ(s)) to reduce undesirable host immune responses to the transplanted tissue. In an additional aspect, fusion proteins can be administered or otherwise delivered to a host to treat one or more disorders associated with transplant tolerance.

Fusion proteins of the invention also can be used to treat immunoproliferative diseases, immunodeficiency diseases, autoimmune diseases, inflammatory responses, and/or allergic responses.

Fusion proteins also can be used to treat proliferative disorders that are non-cancerous or associated with a pre-cancerous condition. For example, fusion proteins can be used to treat one or more proliferative disorders selected from hyperplasias, fibrosis, angiogenesis, psoriasis, atherosclerosis, stenosis or restenosis following angioplasty, and other diseases characterized by smooth muscle proliferation in blood vessels.

The compositions of the invention can be administered in any suitable dosage regimen and by any suitable route and form of administration. Suitability with respect to dosage and administration regimens refers to the administration of any number of doses of a composition, any number of times in a relevant period (typically a day), by any suitable route(s), that result in a desired physiological effect. Dosage regimens may be adjusted to provide the optimum desired response (e.g., a therapeutic or prophylactic response). For example, a single bolus may be administered, several divided doses may be administered over time, or the dose may be proportionally reduced or increased as indicated by the exigencies of the therapeutic situation. It can be especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the fusion protein, related composition, or combination and (b) the particular therapeutic or prophylactic effect to be achieved. The total time of a course of treatment also can be any suitable time and also is likely to vary with a number of similar factors that will be determinable to skilled practitioners with routine experimentation.

As described above, compositions of the invention may include a “therapeutically effective amount” or a “prophylactically effective amount” of a fusion protein (or first and second amounts in the case of a combination composition comprising a fusion protein and a second component; first, second, and third amounts in the case of a combination composition comprising two fusion proteins and a secondary agent or a fusion protein and two secondary agents; etc.).

In practicing the invention, the amount or dosage range of the fusion protein employed typically is one that effectively activates effector lymphocytes, such as NK cells (detectably and desirably significantly promotes, induces, and/or enhances such activation) against target cells.

An exemplary, non-limiting range for a therapeutically or prophylactically effective amount of an antibody or antibody portion of the invention is about 0.1-100 mg/kg, such as about 0.1-50 mg/kg, for example about 0.1-20 mg/kg, and more particularly about 1-10 mg/kg (e.g., at about 0.5 mg/kg (such as 0.3 mg/kg), about 1 mg/kg, or about 3 mg/kg). Generally, such an amount is administered once per day or less (e.g., 2-3 times per week, 1 times per week, or 1 time every two weeks).

For example, an antibody present in a pharmaceutical composition of this invention can be supplied at a concentration of about 10 mg/mL in either about 100 mg (10 mL) or about 500 mg (50 mL) single-use vials. The product can be formulated for IV administration in, e.g., about 9.0 mg/mL sodium chloride, about 7-7.5 mg/mL sodium citrate dihydrate, about 0.7 mg/mL polysorbate 80, and Sterile Water for Injection. The pH typically is adjusted to about 6.5. An exemplary suitable dosage range for a fusion protein antibody in a pharmaceutical composition may between about 10 mg/m² and about 500 mg/m². However, it will be appreciated that these schedules are exemplary and that an optimal schedule and regimen can be adapted taking into account the affinity and tolerability of the particular antibody in the pharmaceutical composition that must be determined in clinical trials. Quantities and schedule of injection of an antibody in a pharmaceutical composition of this invention that saturate NK cells for about 24 hours, about 48 hours, about 72 hours, about a week, or about a month can be determined considering the affinity of the fusion protein antibody and the its pharmacokinetic parameters.

In another aspect, a typical dosage for fusion proteins can range from about 0.01 μg/kg body weight to about 15 mg/kg body weight, such as between about 0.05 μg/kg and about 10 mg/kg body weight, more specifically between about 1 μg/kg and about 10 mg/kg body weight, and even more particularly between about 10 μg/kg and about 5 mg/kg body weight.

In still another aspect, a daily dosage of active ingredient (e.g., fusion protein) of about 0.01 to 100 milligrams per kilogram of body weight is provided to a patient. Ordinarily, about 1 to about 5 or about 1 to about 10 milligrams per kilogram per day given in divided doses of about 1 to about 6 times a day or in sustained release form may be effective to obtain desired results.

As a non-limiting example, treatment of effector lymphocyte-associated pathologies in humans or animals can be provided by administration of a daily dosage of fusion protein(s), such as fusion protein antibodies, in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20, or any combination thereof, using single or divided doses of every about 24, 12, 8, 6, 4, or 2 hours, or any combination thereof.

In general, fusion proteins of the invention can be delivered by any suitable manner, such as by expression from a nucleic acid that codes for production of the fusion protein in target host cells (e.g., by expression from a fusion protein-encoding nucleic acid under the control of an inducible promoter and comprised in a suitable gene transfer vector, such as a targeted and replication-deficient gene transfer vector). Typically, fusion proteins of the invention are delivered by direct administration of the fusion protein or fusion protein composition to a recipient host. In general and where appropriate, the terms “administration” and “delivery” should be construed as providing support for one another herein (e.g., it should generally be recognized that fusion protein-encoding nucleic acids can be used to deliver naked fusion proteins to target host tissues as an alternative to administration of fusion protein proteins), although it also should be recognized that each such method is a unique aspect of the invention with respect to any particular molecule and that some molecules (e.g., fusion protein conjugates) are amenable to only certain forms of administration (as opposed to, e.g., delivery by gene expression). Methods for the administration of proteins, such as antibodies, and related compositions (e.g., vectors), are known and, accordingly, only briefly described here.

Fusion protein compositions, related compositions, and combination compositions can be administered via any suitable route, such as an oral, mucosal, buccal, intranasal, inhalable, intravenous, subcutaneous, intramuscular, parenteral, intertumor, intratumor, or topical route. Such proteins may also be administered continuously via a minipump or other suitable device.

A fusion protein generally will be administered for as long as the disease condition is present, provided that the antibody causes the condition to stop worsening or to improve. A fusion protein will generally be administered as part of a pharmaceutically acceptable composition as described elsewhere herein.

A fusion protein may also be administered or otherwise delivered prophylactically to prevent a disease, disorder, or condition for which such treatment may be effective. For example, fusion proteins can be administered or otherwise delivered to a patient in remission from a cancerous condition in order to reduce the risk of developing cancer, delay the onset of the occurrence of an event in cancer progression, and/or reduce the risk of recurrence of the cancerous condition. This may be especially useful in patients wherein it is difficult to locate a tumor that is known to be present due to other biological factors.

In general, a fusion protein of the invention (or related composition such as a vector comprising a fusion protein-encoding nucleic acid) may be administered by any suitable route, but typically is administered parenterally in dosage unit formulations containing conventional pharmaceutically acceptable carriers, adjuvants, and the like (stabilizers, disintegrating agents, anti-oxidants, etc.). The term “parenteral” as used herein includes, subcutaneous, intravenous, intraarterial, intramuscular, intrasternal, intratendinous, intraspinal, intracranial, intrathoracic, infusion techniques and intraperitoneal delivery. Most commonly, a fusion protein will be administered intravenously or subcutaneously, in practicing therapeutic methods of the invention. Routes of injection also include injection into the muscle (intramuscular IM); injection under the skin (subcutaneous (s.c.)); injection into a vein (intravenous (IV)); injection into the abdominal cavity (intraperitoneal (IP)); and other delivery into/through the skin (intradermal delivery, usually by multiple injections, which may include biolistic injections).

As described above, the invention provides a number of combination compositions and combination methods. The dose and route of delivery of each of the fusion protein and secondary agent can be any suitable dosage and route for achieving the desired therapeutic, prophylactic, and/or physiological effects in the recipient host (e.g., activation of NK cells; neutralization of NK cell inhibition; or reduction in the number of tumors in a patient). In view of the combined effects of the fusion protein and secondary agent in such methods and compositions, the dosage of the fusion protein typically is lowered in such methods and compositions.

In general, combination administration methods of the invention can comprise any suitable administration scheme, including coadministration (as separate compositions or a single composition wherein the ingredients are mixed or separated) or stepwise administration of the various active agents.

The terms “coadministration,” “coadminister,” and the like herein refer to both to simultaneous administration (or concurrent administration) and serial but related administration, unless otherwise indicated. Coadministration of agents can be accomplished in any suitable manner and in any suitable time. In other words, coadministration can refer to administration of a fusion protein before, simultaneously with, or after, the administration of a secondary agent, at any time(s) that result(s) in an enhancement in the therapeutic response over the administration of solely the secondary agent, fusion protein, or both agents independently.

When one or more agents are used in combination with fusion protein of this invention in a therapeutic regimen, there is no requirement for the combined results to be additive of the effects observed when each treatment is conducted separately. Although at least additive effects are generally desirable, any increased anti-cancer effect above one of the single therapies would be of benefit. Also, there is no particular requirement for the combined treatment to exhibit synergistic effects, although this is certainly possible and advantageous.

To practice combined anti-cancer therapy, for example, one can simply administer to a mammal or other suitable animal an antibody composition of this invention in combination with another anti-cancer agent or method in a manner effective to result in their combined anti-cancer actions within the animal. The agents or agent and method would therefore be provided or applied in amounts effective and for periods of time effective to result in a combined effect against the tumor or other cancer-associated tissues. To achieve this goal, a fusion protein of this invention and one or more secondary anti-cancer agents may be administered to the animal simultaneously, either in a single combined composition, or as two distinct compositions using different administration routes. Alternatively, the administration of a fusion protein of this invention may precede, or follow, the anti-cancer agent treatment by, e.g., intervals ranging from minutes to weeks and months. One would ensure that the secondary anti-cancer agent and fusion protein in the composition of this invention exert an advantageously combined effect on the cancer.

Different therapeutic regiments involving fusion proteins and combination compositions can be applied with respect to different aspects of various disease targets, such as cancer treatment and the treatment of viral infections. Thus, for example, in one aspect a fusion protein is delivered to a patient as part of an anti-initiation strategy (in the context of the treatment of cancer). Advantageous secondary antineoplastic agents and techniques for administration, delivery, or application in the context of an anti-initiation therapeutic regimen include, for example, DNA repair enzymes, molecules that scavenge for reactive oxygen species and electrophiles, and compositions that enhance carcinogen detoxification. In another aspect, a fusion protein, related composition, or combination composition is delivered, administered, or applied in the context of an anti-promotion and/or anti-proliferation therapeutic regimen. Advantageous secondary agents and techniques in the context of such a therapeutic regimen include, for example, agents and techniques that induce cancer cell death, agents and techniques that suppress cancer cell proliferation (e.g., chemotherapeutic agents), and agents that alter cancer cell-associated gene expression (e.g., agents that reduce expression of cancer-promoting genes, methods that involve re-introducing functional tumor suppressors, etc.).

In an exemplary combinatorial treatment aspect, the invention provides a method of treating a cancerous or precancerous condition that comprises application of radiation or associated administration of radiopharmaceuticals to a patient (combination compositions comprising radiopharmaceuticals is another feature of the invention). The source of radiation can be either external or internal to the patient being treated (radiation treatment can, for example, be in the form of external beam radiation therapy (EBRT) or brachytherapy (BT)). Radioactive elements that can be used in practicing such methods include, e.g., radium, Cesium-137, Iridium-192, Americium-241, Gold-198, Cobalt-57, Copper-67, Technetium-99, Iodide-123, Iodide-131, and Indium-111. Additionally useful radionuclides that can be incorporated in radiopharmaceuticals and used in such methods are discussed elsewhere herein.

As described above, a fusion protein and/or related compound can be administered in association with one or more suitable anti-cancer and cancer preventative agents. In other aspects, a fusion protein (e.g., a fusion protein antibody) and/or related composition is administered in association with a thrombosis modulating agent such as a low molecular weight heparin, standard heparin, pentasaccharides, thrombin inhibitory agents (melagatran, ximelagatran, etc.), and/or coagulation factors like Factor VII, Factor VIII, etc. In a further aspect, a fusion protein is administered in association with syngeneic and/or allogeneic stem cell transplantation. In a further aspect, fusion proteins are administered in association with an anti-cancer gene therapy protocol, which can include administration of vectors expressing one or more anti-cancer genes, such as DNA vaccines encoding cancer antigens, adenoviral vectors encoding anti-cancer cytokines, or the like. Fusion proteins also can be administered or otherwise delivered in association with inhibitory nucleic acid therapy such as an anti-cancer siRNA.

### Production of Fusion Proteins

In another aspect, the invention provides methods for producing a fusion protein comprising antibody and target-binding portions.

In one aspect, the invention provides such a method wherein the method comprises providing a first nucleic acid comprising a sequence that encodes a antibody portion, attaching it in frame to a second nucleic acid comprising a sequence encoding a target-binding to form a third fused nucleic acid, such that expression of the fused nucleic acid leads to production of the protein, transfecting a cell that is able to express the fused nucleic acid with the fused nucleic acid, and maintaining the cell under conditions suitable for expression of the fusion protein. In a variation on this aspect, a nucleic acid encoding the fusion protein may be designed and synthesized, without the requirement for fusion of separate nucleic acids.

The “cell” in the above-described method may refer to a cell in culture or refer to a cell contained in a vertebrate host.

A nucleic acid encoding a antibody portion of a fusion protein may be obtained by selecting an antibody against an effector lymphocyte activating receptor, sequencing the antibody or a functional portion thereof, and preparing a nucleic acid sequence that encodes the antibody or functional portion. Antibodies may be screened for receptor binding by various known methods such as by ELISA or phage display methods. Receptor activation also can be determined by standard methods, if desired, which will vary with the type of receptor at issue. Activation can be measured by effector lymphocyte activation (e.g., by cell proliferation, cell-associated cytokine production, etc.) or by more receptor-specific methods (if available), such as measurement of the production of a component of a receptor-associated pathway in associated cells. Techniques for making such assessments are known.

A nucleic acid sequence encoding functional antibody and/or target-binding sequences may be subjected to various select or random modifications to produce variants of such sequences as par of a method of producing fusion proteins of the invention. Thus, in one aspect, the invention provides a method of producing fusion proteins comprising anti-body and target-binding portions that comprises obtaining such sequences and subjecting one or both to modification by introducing one or more additions, substitutions, deletions, insertions, or combinations thereof into the nucleic acid sequence(s).

Production of fusion proteins of this invention can also involve the production and expression of multiple nucleic acids in a cell, particularly where one or more portions of the fusion protein is/are in the form of a multimeric protein structure, such as an antibody or anti-body fragment. For example, where the target-binding portion is on a single protein chain and the antibody portion is contained in two chains (e.g., a heavy chain and light chain portion such as in an antibody), the inventive method may comprise generating a nucleic acid encoding a fused peptide that comprises the heavy chain antibody sequence (or variant) (e.g., an anti-CD16 heavy chain sequence) fused (directly or indirectly) to the target-binding portion (e.g., an NKG2D extracellular domain), which is transfected into a cell in combination with a nucleic acid sequence encoding a peptide comprising a light chain portion of the anti-CD16 antibody, so as to form the fusion protein upon expression of both nucleic acids. Of course, a single nucleic acid comprising separated sequences may alternatively be used in such situations.

### EXAMPLES

The following exemplary experimental methods and data are presented to better illustrate various aspects of the invention, but in no event should be viewed as limiting the scope of the invention.

### Example 1

The following example describes the production of a fusion protein comprising a anti-aCD3 portion and an NKG2D portion.

cDNA encoding an anti-mouse CD3 antibody was cloned from a hamster anti-mouse CD3 producing cell line (145-2c11). The total RNA was purified according to manufacturers instructions (RNeasy from Qiagen, VWR, Denmark) and the gene sequences amplified using specific primers in a RT-PCR reaction using SuperScript™ III One-Step RT-PCR System with Platinum® Taq DNA Polymerase from Invitrogen and the reaction cycles: [37° C. 30 min][94° C.] min] 25×[94° C. 30 s; 55° C. 30 s; 72° C. min][72° C. 5 min]. The RT-PCR products were analyzed by electrophoresis on a 1% agarose gel and the DNA purified from the gel using GFX PCR and Gel Band Purification Kit (Amersham Biosciences, Denmark). The primers used to obtain the anti-mouse CD3 light chain were oligonucleotides oVWS109 and oVWS110.

oVWS109 (145.2c11 light forward) (SEQ ID NO:22):
5′ ATGAGGGCCCCTACTGTGTATCC 3′
oVWS110 (145.2c11 light reverse) (SEQ ID NO:23):
5′ GGACCTCTGGCTCTAACACTCATTCC 3′

The cDNA was introduced into a pCR 2.1-TOPO vector using TOPO TA Cloning kit from Invitrogen and transformed into TOP10 competent cells. The DNA sequence was confirmed by sequencing using primer M13 forward and M13reverse resulting in the anti-mouse CD3 light chain.

M13 forward primer (SEQ ID NO:24):
5′ GTAAAACGACGGCCAG 3′
M13 reverse primer (SEQ ID NO:25):
5′ CAGGAAACAGCTATGAC 3′
Anti-mouse CD3 light chain (SEQ ID NO:26):
Signal sequence and variable + constant region
underlined
AGGGCGAATTGGGCCCTCTAGATGCATGCTCGAGCGGCCGCCAGTGTGAT
GGATATCTGCAGAATTCGCCCTTATGAGGGCCCCTACTGTGTATCCTGTG
CTCTTGTTTCTTTGGTTTACAGGTGCCATATGTGACATCCAGATGACCCA
GTCTCCATCATCACTGCCTGCCTCCCTGGGAGACAGAGTCACTATCAATT
GTCAGGCCAGTCAGGACATTAGCAATTATTTAAACTGGTACCAGCAGAAA
CCAGGGAAAGCTCCTAAGCTCCTGATCTATTATACAAATAAATTGGCAGA
TGGAGTCCCATCAAGGTTCAGTGGCAGTGGTTCTGGGAGAGATTCTTCTT
TCACTATCAGCAGCCTGGAATCCGAAGATATTGGATCTTATTACTGTCAA
CAGTATTATAACTATCCGTGGACGTTCGGACCTGGCACCAAGCTGGAAAT
CAAACGGGCTGATGCTAAGCCAACCGTCTCCATCTTCCCACCATCCAGTG
AGCAGTTGGGCACTGGAAGTGCCACACTTGTGTGCTTCGTGAACAACTTC
TACCCCAAAGACATCAATGTCAAGTGGAAAGTAGATGGCAGTGAAAAACG
AGATGGCGTCCTGCAGAGTGTCACTGATCAGGACAGCAAAGACAGCACCT
ACAGCCTGAGCAGCACCCTCTCGCTGACCAAAGCAGATTATGAGAGGCAT
AACCTGTATACCTGTGAGGTTACTCATAAGACATCAACTGCAGCCATTGT
CAAGACCCTGAACAGGAATGAGTGT TAGAGCAGAGGTCCAAGGGCGAAT
TCCAGCACACTGGCGGC-CGTTACT

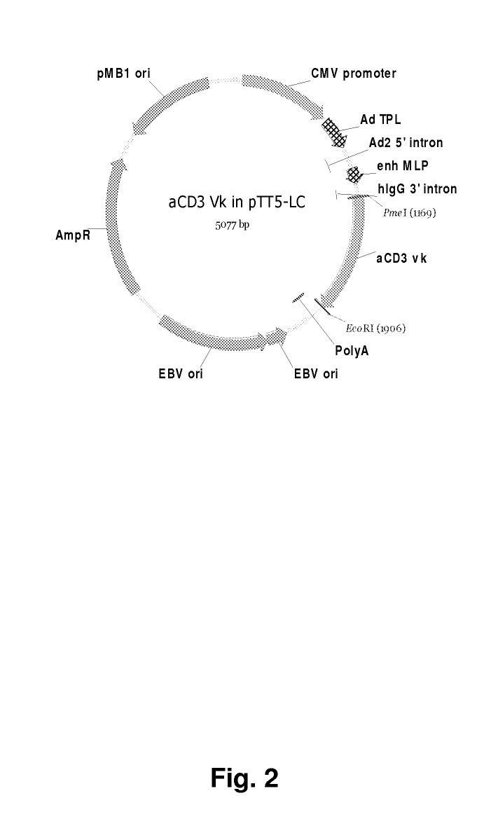

The DNA encoding the anti CD3 light chain was digested with the restriction enzymes PmeI and EcoRI and ligated into the corresponding sites in the mammalian expression vector pTT5-LC (FIG. 2) and the sequence confirmed by sequencing the plasmid using primers oVWS121 and oVWS122.

oVWS 121 (SEQ ID NO:27):
5′ GTACTCCCTCTCAAAAGCGGGC 3′
oVWS 122 (SEQ ID NO:28):
5′ CTGAAGGGATTACATGCACTGCCC 3′

To obtain the variable region of the heavy chain the sequence specific oligonucleotides oVWS106 and oVWS108 were used in a RT-PCR reaction using SuperScript™ III One-Step RT-PCR System with Platinum® Taq DNA Polymerase from Invitrogen and the reaction cycles: [37° C. 30 min][94° C. 1 min] 25×[94° C. 30 s; 55° C. 30 s; 72° C. 1 min][72° C. 5 min]. The RT-PCR products were analyzed by electrophoresis on a 1% agarose gel and the DNA purified from the gel using GFX PCR and Gel Band Purification Kit (Amersham Biosciences, Denmark) and introduced into pCR 2.1-TOPO vector using TOPO TA Cloning kit from Invitrogen and transformed into TOP10 competent cells. The DNA sequence was confirmed by sequencing using primer M13 forward and primer M13reverse resulting in the sequence encoding anti-mouse CD3 heavy chain:

Hamster anti-mouse CD3 heavy chain (SEQ ID NO:29):
Signal sequence and variable region underlined
TATACGACTCACTATAGGGCGAATTGGGCCCTCTAGATGCATGCTCGAGC
GGCCGCCAGTGTGATGGATATCTGCAGAATTCGCCCTTCAGCCCTGGATT
CCCAGGTCCTCCCATTCTGTGATCAGCACTGAACACAGGCCACTTGCCAT
GAACTCAGGACTCCAATTGGTTTTCTTTGTCCTCACTCTAAAAGGTATAC
AGGGTGAGGTGCAGCTGGTGGAGTCTGGGGGAGGCTTGGTGCAGCCTGGA
AAGTCCCTGAAACTCTCCTGTGAGGCCTCTGGATTCACCTTCAGCGGCTA
TGGCATGCACTGGGTCCGCCAGGCTCCAGGGAGGGGGCTGGAGTCGGTCG
CATACATTACTAGTAGTAGTATTAATATCAAATATGCTGACGCTGTGAAA
GGCCGGTTCACCGTCTCCAGAGACAATGCCAAGAACTTACTGTTTCTACA
AATGAACATTCTCAAGTCTGAGGACACAGCCATGTACTACTGTGCAAGAT
TCGACTGGGACAAAAATTACTGGGGCCAAGGAACCATGGTCACCGTCGCC
TCAGCCAAAACAACAGCCCCAAAGGGCGAATTCCAGCACACTGGCGGCCG
TTACTAGTGGATCCGAGCTCGGTACCAAGCTTGGCGTAATCATGGTCATA
GCTGTTTCCTGTGTGAAATTGTTATCCGCTCACAATTCCACACAACATAC
GAGCCGGAAGCATAAAGTGTAAAGCCTGGGGTGCCTAATGAGTGAGCTAA
CTCACATTAATTGCGTTGCGCTCACTGCCCGCTTTCCAGTCGGGAAACCT
GTCGTGCCAGCTGCATTAATGAATCGGCCAACGCGCGGGGAGAGGCGGTT
TGCGTATTGGGCGCTCTTCCGCTTCCTCGCTCACTGACTCGCTGCGCTCG
GTCGTTCGGCTGCGGCGAGCGGTATCAGCTCA
oVWS 106 (145.2c11 heavy forward) (SEQ ID NO:30):
5′ CAGCCCTGGATTCCCAGGTCCTC 3′
oVWS108 (145.2c11 heavy reverse) (SEQ ID NO:31):
5′ TGGGGCTGTTGTTTTGGCTGAGGAG 3′

Because 145.2c11 is a hamster antibody and a murine construct was desired, the hamster Fc portion was shuffled with the corresponding murine Fc sequence (SEQ ID NO:32) by enzymatic digests and ligations.

Mus musculus immunoglobulin heavy chain 4 (serum
IgG1) (SEQ ID NO:32):
The cloned (murine) Fc portion is underlined.
CTGGGGATGCTGGTCGCTTCCGTGCTAGCAGTGCCCAGGGATTGTGGTTG
TAAGCCTTGCATATGTACAGTCCCAGAAGTATCATCTGTCTTCATCTTCC
CCCCAAAGCCCAAGGATGTGCTCACCATTACTCTGACTCCTAAGGTCACG
TGTGTTGTGGTAGACATCAGCAAGGATGATCCCGAGGTCCAGTTCAGCTG
GTTTGTAGATGATGTGGAGGTGCACACAGCTCAGACAAAACCCCGGGAGG
AGCAGTTCAACAGCACTTTCCGTTCAGTCAGTGAACTTCCCATCATGCAC
CAGGACTGGCTCAATGGCAAGGAGTTCAAATGCAGGGTCAACAGTGCAGC
TTTCCCTGCCCCCATCGAGAAAACCATCTCCAAAACCAAAGGCAGACCGA
AGGCTCCACAGGTGTACACCATTCCACCTCCCAAGGAGCAGATGGCCAAG
GATAAAGTCAGTCTGACCTGCATGATAACAGACTTCTTCCCTGAAGACAT
TACTGTGGAGTGGCAGTGGAATGGGCAGCCAGCGGAGAACTACAAGAACA
CTCAGCCCATCATGGACACAGATGGCTCTTACTTCGTCTACAGCAAGCTC
AATGTGCAGAAGAGCAACTGGGAGGCAGGAAATACTTTCACCTGCTCTGT
GTTACATGAGGGCCTGCACAACCACCATACTGAGAAGAGCCTCTCCCACT
CTCCTGGTAAAGGATCCTTATTCA

A Kozak sequence was introduced upstream of the fusion protein-encoding sequence by standard PCR using an oligonucleotide comprising sequence oVWS116:

oVWS 116 (SEQ ID NO:33):
5′ TAACATCCTTGAATTCACTTGCCACCATGAACTCAGGACTCCAATTG
G 3′

The ectodomain of murine NKG2D was amplified by PCR using the clone ID 5328432 from OpenBiosystems (AL, USA) as template and specific primers.

mNKG2D ec forward (SEQ ID NO:34):
5′ GTTGAGAATCAGCTGTGCAACAAGGAAGTCCC 3′
mNKG2D ec reverse (SEQ ID NO:35):
5′ TAACCATGGCGGCCGTTTTTACACCGCCCTTTTCATGC 3′

The resulting cDNA was cloned into pCR 2.1-TOPO vector using TOPO TA Cloning kit from Invitrogen and transformed into TOP10 competent cells resulting in clone mNKG2D_D1. Plasmid DNA was extracted and sequenced using primer M13 forward M13 reverse resulting in the following sequence. The portion of the sequence encoding the ectodomain of mNKG2D is underlined.

Sequence of mNKG2D_D1

(SEQ ID NO:36)

GCCTTTTGCTCACATGTTCTTTCCTGCGTAATCCCCTGATTCTGTGGATA
ACCGTATTACCGCCTTTGAGTGAGCTGATACCGCTCGCCGCAGCCGAACG
ACCGAGCGCAGCGAGTCAGTGAGCGAGGAAGCGGAAGAGCGCCCAATACG
CAAACCGCCTCTCCCCGCGCGTTGGCCGATTCATTAATGCAGCTGGCACG
ACAGGTTTCCCGACTGGAAAGCGGGCAGTGAGCGCAACGCAATTAATGTG
AGTTAGCTCACTCATTAGGCACCCCAGGCTTTACACTTTATGCTTCCGGC
TCGTATGTTGTGTGGAATTGTGAGCGGATAACAATTTCACACAGGAAACA
GCTATGACCATGATTACGCCAAGCTTGGTACCGAGCTCGGATCCACTAGT
AACGGCCGCCAGTGTGCTGGAATTCGCCCTTGTTGAGAATCAGCTGTGCA
ACAAGGAAGTCCCAGTTTCCTCAAGAGAGGGCTACTGTGGCCCATGCCCT
AACAACTGGATATGTCACAGAAACAACTGTTACCAATTTTTTAATGAAGA
GAAAACCTGGAACCAGAGCCAAGCTTCCTGTTTGTCTCAAAATTCCAGCC
TTCTGAAGATATACAGTAAAGAAGAACAGGATTTCTTAAAGCTGGTTAAG
TCCTATCACTGGATGGGACTGGTCCAGATCCCAGCAAATGGCTCCTGGCA
GTGGGAAGATGGCTCCTCTCTCTCATACAATCAGTTAACTCTGGTGGAAA
TACCAAAAGGATCCTGTGCTGTCTATGGCTCAAGCTTTAAGGCTTACACA
GAAGACTGTGCAAATCTAAACACGTACATCTGCATGAAAAGGGCGGTGTA
AAAACGGCCGCCATGGTTAAAGGGCGAATTCTGCAGATATCCATCACACT
GGCGGCCGCTCGAGCATGCATCTAGAGGGCCCAATTCGCCCTATAGTGAG
TCGTA

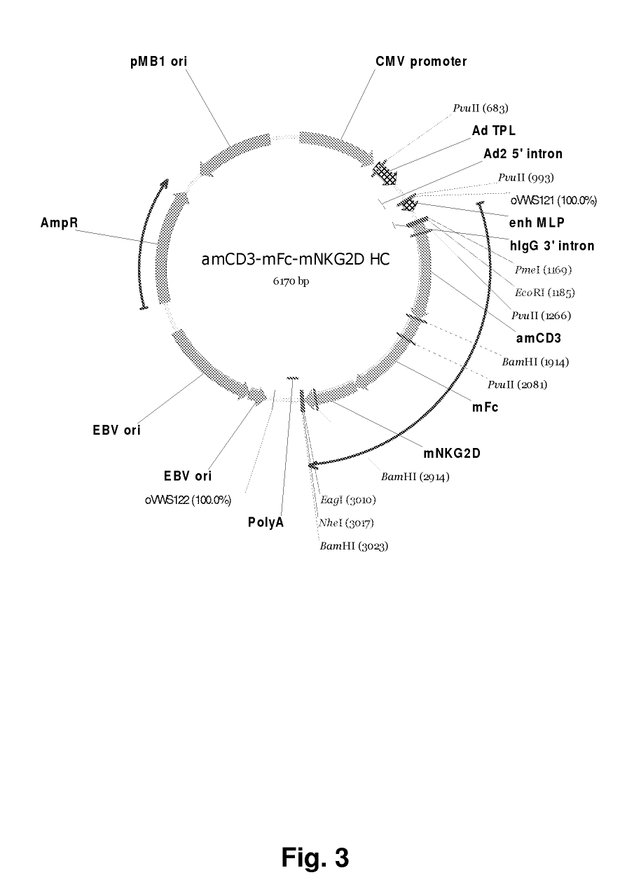

The three parts (antiCD3 heavy chain—murine Fc part—ectodomain of mNKG2D) were ligated and cloned into mammalian expression vector (pTT5-HC) (FIG. 3) and the sequence confirmed by sequencing primers oVWS121 and oVWS122.

The two plasmids (aCD3Vk in pTT5-LC (see FIG. 2) and amCD3-mFc-mNKG2D HC (FIG. 3)) were co-expressed in mammalian cell lines (Cos-7, HEK293 6E) obtain the exemplary fusion protein (other cells, such as CHO cells, may alternatively be advantageously used). The fusion protein was secreted into the media and purified on a Protein A column and used in various assays. The amino acid sequence of the heavy chain and light chains of the aCD3-mFc-mNKG2D construct are shown here:

(heavy chain - SEQ ID NO:37)

MNSGLQLVFFVLTLKGIQGEVQLVESGGGLVQPGKSLKLSCEASGFTFSG
YGMHWVRQAPGRGLESVAYITSSSINIKYADAVKGRFTVSRDNAKNLLFL
QMNILKSEDTAMYYCARFDWDKNYWGQGTMVTVSSATTTAPSVYPLAPAC
DSTTSTTNTVTLGCLVKGYFPEPVTVSWNSGALTSGVHTFPSVLHSGLYS
LSSSVTVPSSTWPSQTVTCNVAHPASSTKVDKKIVPRGDPRDCGCKPCIC
TVPEVSSVFIFPPKPKDVLTITLTPKVTCVVVDISKDDPEVQFSWFVDDV
EVHTAQTKPREEQFNSTFRSVSELPIMHQDWLNGKEFKCRVNSAAFPAPI
EKTISKTKGRPKAPQVYTIPPPKEQMAKDKVSLTCMITDFFPEDITVEWQ
WNGQPAENYKNTQPIMDTDGSYFVYSKLNVQKSNWEAGNTFTCSVLHEGL
HNHHTEKSLSHSPGKDLCNKEVPVSSREGYCGPCPNNWICHRNNCYQFFN
EEKTWNQSQASCLSQNSSLLKIYSKEEQDFLKLVKSYHWMGLVQIPANGS
WQWEDGSSLSYNQLTLVEIPKGSCAVYGSSFKAYTEDCANLNTYICMK
RAV

(light chain SEQ ID NO:38)

MRAPTVYPVLLFLWFTGAICDIQMTQSPSSLPASLGDRVTINCQASQDIS
NYLNWYQQKPGKAPKLLIYYTNKLADGVPSRFSGSGSGRDSSFTISSLES
EDIGSYYCQQYYNYPWTFGPGTKLEIKRADAKPTVSIFPPSSEQLGTGSA
TLVCFVNNFYPKDINVKWKVDGSEKRDGVLQSVTDQDSKDSTYSLSSTLS
LTKADYERHNLYTCEVTHKTSTAAIVKTLNRNEC

The heavy chain construct sequence (SEQ ID NO:37) comprises the following segments:

M1 to G19: signal sequence from anti-CD3 (which is cleaved of the purified construct);

E20 to V133: anti-CD3 variable region;

S134 to 1234: Ig heavy chain constant region (part of the anti CD3);

V235 to D462: mouse Fc part; and

L463 to V597: ectodomain of murine NKG2D.

This Example demonstrates a method by which fusion proteins having features associated with aspects of the invention can be produced. Skilled artisans will be able to produce fusion proteins having similar combinations of features using similar techniques or acceptable alternatives thereof given the disclosure provided herein. Fusion proteins comprising such heavy and light chains or antibody sequences similar thereto (e.g., functional fragments thereof or highly similar variants thereof (e.g., sequences having at least about 75%, 80%, 85%, 90%, or 95% identity thereto and exhibiting NKG2D binding) are additional particular features of this invention.

### Example 2

This example demonstrates a method by which the functionality of an exemplary fusion protein of the invention may be assessed.

The functionality of the fusion protein produced in Example 1 was assessed in vitro by Cr51 release assay. Cells carrying a NKG3D ligand such as MicA (e.g., HEK293 cells) were used as target cells and cytotoxic T cells that do not express a NKG2D ligand as effector cells. The target cells are incubated in a solution containing a radioactive isotope of chromium, chromium 51 (Cr51). Cr51 is spontaneously taken up into the cells and stored in the cytosol, and excess chromium containing solution is washed away. Activated CD8 cells are then added to the cell-containing media, and both cell types are incubated together.

During this period of co-incubation, the activated CD8 lymphocytes will eventually recognize the target cells and cause cell lysis. As the cells lyse, the chromium that they had taken up is released into the supernatant of the mixture. The sample is then centrifuged to pellet the remaining cells and excess cellular debris, and the supernatant containing the chromium is isolated. The amount of chromium that is released from the cells determines the effectivity of the cytotoxicity of the CD8 cells. In order to determine definitive quantitative data however, appropriate control experiments such as an experiment with no CD8 lymphocytes must be performed in order to assay the amount of spontaneous release of Cr51 from the experimental infected cells.

The CD8+ T cells require two stimuli for activation (T cell receptor and CD28) and even though NKG2D is expressed on CD8+ T cells, NKG2D binding itself is not sufficient to stimulate proliferation or effector function (Ehrlich et al. J. Immunol. 2005; 174(4):1922-31). Thus, any background cell killing will be largely overcome when adding the bispecific molecule. Moreover, because the fusion protein is made from murine proteins it is also possible to test the concept in mice. Such testing is expected to confirm the ability of the fusion protein to promote killing of target cells. Moreover, this example demonstrates how other fusion proteins produced according to the inventive methods set forth herein may be evaluated.

### Example 3

This example describes how to produce a fusion protein comprising a human anti-αCD3 portion and a human NKG2D portion.

In order to make the fusion protein, total RNA is purified according to manufacturers instructions (RNeasy from Qiagen, VWR, Denmark) from a hybridoma cell line, OKT3, expressing the mouse monoclonal antibody against human T cell CD3.

The cDNAs of the variable heavy (VH) and variable light (VL) chains are amplified by polymerase chain reaction (PCR) method using the SMART RACE (Rapid Amplification of cDNA Ends) cDNA Amplification Kit from Clontech (BD Bioscience, Denmark) according to manufacturers instructions using 1 μg of the purified RNA (described above), the 5′ RACE CDS primer and BD SMART II A oligo.

5′ RACE CDS primer:
5′ (T)25VN 3′ (V = A,G, og C; N = A,C,G, or T)
BD SMART II A oligo (SEQ ID NO:39):
5′ AAGCAGTGGTATCAACGCAGAGTACGCGGG 3′

The VH and VL regions of OKT3 cDNA (made as described above) are amplified by PCR according to the manufacturer using the following primers:

Universal Primer A Mix (UPM)-Long (SEQ ID NO:40):
5′ CTAATACGACTCACTATAGGGCAAGCAGTGGTATCAACGCAGAG
T 3′
Universal Primer A Mix (UPM)-Short (SEQ ID NO:41):
5′ CTAATACGACTCACTATAGGGC 3′
KK185 for VL chain (SEQ ID NO:42):
5′ GGCCCGGGGGGCCTTAACACTCATTCCTGTTGAAGCT
KK229 for VH chain (SEQ ID NO:43):
5′-TTATTTACCAGGAGAGTGGGAGAG
KK230 for VH chain (SEQ ID NO:44):
5′-TCATTTACCCAGAGACCGGGAG
KK231 for VH chain (SEQ ID NO:45):
5′-TCATTTACCCGGAGACCGGGAG

The PCR products are analyzed by electrophoresis on a 1% agarose gel and the DNA purified from the gel using GFX PCR and Gel Band Purification Kit (Amersham Biosciences, Denmark).

The purified PCR products are introduced into pCR 2.1-TOPO vector using TOPO TA Cloning kit from Invitrogen and transformed into TOP10 competent cells.

More than 15 colonies are analyzed by colony PCR using Taq polymerase, 1×Taq polymerase buffer, dNTP (10 mM) and the following primers and PCR program:

PCR Program 1:

[94° C. 1 min] 25×[94° C. 30 s; 55° C. 30 s; 72° C. 1 min][72° C. 5 min] Plasmid DNA from clones comprising VL and VH inserts, respectively, is extracted and sequenced using primer M13 forward (SEQ No 3) and M13reverse (SEQ No 4) listed above.

The resulting sequences for OKT3:

VH (SEQ ID NO:46):
caatgaatgatacgccagcttggtaccgagctcggatccactagtaacgg
ccgccagtgtgctggaattcgcccttctaatacgactcactatagggcaa
gcagtggtatcaacgcagagtacgcggggcttctcttgggtcttagacaa
acgtatccattaaattcaaatattcttctggcagtcaagaaaagaagcag
ccaaaacaacacccccatcagtctatccactggcccctgggtgtggagat
acaactggttcctccgtgactctgggatgcctggtcaagggctacttccc
tgagtcagtgactgtgacttggaactctggatccctgtccagcagtgtgc
acaccttcccagctctcctgcagtctggactctacactatgagcagctca
gtgactgtcccctccagcacctggccaagtcagaccgtcacctgcagcgt
tgctcacccagccagcagcaccacggtggacaaaaaacttgagcccagcg
ggcccatttcaacaatcaacccctgtcctccatgcaaggagtgtcacaaa
tgcccagctcctaacctcgagggtggaccatccgtcttcatcttccctcc
aaatatcaaggatgtactcatgatctccctgacacccaaggtcacgtgtg
tggtggtggatgtgagcgaggatgacccagacgtccagatcagctggttt
gtgaacaacgtggaagtacacacagctcagacacaaacccatagagagga
ttacaacagtactatccgggtggtcagcaccctccccatccagcaccagg
actggatgagtggcaaggagttcaaatgcaaggtcaacaacaaagacctc
ccatcacccatcgagagaaccatctcaaaattaaagggctagtcagagct
ccacaagtatacatcttgccgccaccagcagagcagttgtccaggaaaga
tgtcagtctcacttgcctggtcgtgggcttcaaccctggagacatcagtg
tggagtggacagcatgggcatacggaggagactacagggaccggcacagt
ctggactctgacggtcttactcatttttgcaaggtcatatgaaacaagca
aggggaaaacagaattcttctcagccactgggaaccagggttaaaaatat
caccggagaaaaacccccccggccccgggaaagaaaggggaatttcccaa
attttcccatctggggccctcaaataattaagggccccacccccccaaag
gggaaaaaaataagggggcggttttataccggaggggaaaaagaggtgtg
aaaataatatttgtggaaacccttccccgcgggggagagagaaagagccc
gccacccctcccccccccccgcggggaggagggcgc
VL (SEQ ID NO:47):
gggcgaattgggccctctagatgcatgctcgagcggccgccagtgtgatg
gatatctgcagaattcgcccttaagcagtggtatcaacgcagagtacgcg
gggagagatggagacagacacactcctgttatgggtactgctgctctggg
ttccaggttccactggtgacattgtgctgacacggtctcctgcttcctta
gctgtatctctggggcagagggccaccatctcatacagggccagcaaaag
tgtcagtacatctggctatagttatatgcactggaaccaacagaaaccag
gacagccacccagactcctcatctatcttgtatccaacctagaatctggg
gtccctgccaggttcagtggcagtgggtctgggacagacttcacccccaa
catccatcctgtggaggaggaggatgctgcaacctattactgtcagcaca
ttagggagcttacacgttcggaggggggaccaagctggaaataaaacggg
ctgatgctgcaccaactgtatccatcttcccaccatccagtgagcagtta
acatctggaggtgcctcagtcgtgtgcttcttgaacaacttctaccccaa
agacatcaatgtcaagtggaagattgatggcagtgaacgacaaaatggcg
tcctgaacagttggactgatcaggacagcaaagacagcacctacagcatg
agcagcaccctcacgtcgaccaaggacgagtatgaacgacataacagcta
tacctgtgaggccactcacaagacatcaacttcacccattgtcaagagct
tcaacaggaatgagtgttaaggccccccgggccaagggcgaattccagca
cactggcggccgttactagtggatccgagctcggtaccaagcttggcgta
atcatggtcatagctgtttcctgtgtgaaattgttatccgctcacaattc
cacacaacatacgagccggaagcataagtgtaagcctgggtgcctaatga
gtgagctact

The mouse Fc region of OKT VH is shuffled with the corresponding region of human Fc

Human Fc IgGi (SEQ ID NO:48):
CTCGAGCCCAAATCTTGTGACAAAACTCACACATGCCCACCGTGCCCAGC
ACCTGAACTCCTGGGGGGACCGTCAGTCTTCCTCTTCCCCCCAAAACCCA
AGGACACCCTCATGATCTCCCGGACCCCTGAGGTCACATGCGTGGTGGTG
GACGTGAGCCACGAAGACCCTGAGGTCAAGTTCAACTGGTACGTGGACGG
CGTGGAGGTGCATAATGCCAAGACAAAGCCGCGGGAGGAGCAGTACAACA
GCACGTACCGTGTGGTCAGCGTCCTCACCGTCCTGCACCAGGACTGGCTG
AATGGCAAGGAGTACAAGTGCAAGGTCTCCAACAAAGCCCTCCCAGCCCC
CATCGAGAAAACCATCTCCAAAGCCAAAGGGCAGCCCCGAGAACCACAGG
TGTACACCCTGCCCCCATCCCGGGATGAGCTGACCAAGAACCAGGTCAGC
CTGACCTGCCTGGTCAAAGGCTTCTATCCCAGCGACATCGCCGTGGAGTG
GGAGAGCAATGGGCAGCCGGAGAACAACTACAAGACCACGCCTCCCGTGC
TGGACTCCGACGGCTCCTTCTTCCTCTACAGCAAGCTCACCGTGGACAAG
AGCAGGTGGCAGCAGGGGAACGTCTTCTCATGCTCCGTGATGCATGAGGC
TCTGCACAACCACTACACGCAGAAGAGCCTCTCCCTGTCTCCGGGTAAA
TGA

The cDNA encoding the ectodomain hNKG2D is performed using Human Spleen Marathon Ready cDNA (Clontech) and hNKG2D specific primers

hNKG2D upstream (SEQ ID NO:49):
5′ AGTATTTGATGGGGTGGATTCG 3′
hNKG2D downstream (SEQ ID NO:50):
5′ GATGGTTGATCATCTTTACACAGT 3′

After PCR reactions using the above-mentioned primers and essentially the PCR program 1 mentioned above, the ectodomain-encoding cDNA is further amplified using primers (below) introducing restriction enzyme sites for further cloning. The resulting cDNA is cloned into pCR 2.1-TOPO vector using TOPO TA Cloning kit from Invitrogen and trans-formed into TOP10 competent cells. Plasmid DNA is extracted and sequenced using primers M13 forward and M13 reverse resulting in isolation of a sequence encoding the ectodomain of NKG2D.

Extracellular hNKG2D upstream (SEQ ID NO:51):
CGTTGCGCTGCTAGCCGTTGCGCTGGATCCTTATTCAACCAAGAAGTT-C
AAATTCCC
Extracellular hNKG2D downstream (SEQ ID NO:52):
5′ CGTTGCGCTCGGCCGTTTACACAGTCCTTTGCATGCAGATGT 3′

Sequence encoding the ectodomain of NKG2D cDNA flanked by the restriction enzyme sites NheI, BamHI and EagI (SEQ ID NO:53):

CGTTGCGCTGCTAGCCGTTGCGCTGGATCCTTATTCAACCAAGAAGTTCA
AATTCCCTTGACCGAAAGTTACTGTGGCCCATGTCCTAAAAACTGGATAT
GTTACAAAAATAACTGCTACCAATTTTTTGATGAGAGTAAAAACTGGTAT
GAGAGCCAGGCTTCTTGTATGTCTCAAAATGCCAGCCTTCTGAAAGTATA
CAGCAAAGAGGACCAGGATTTACTTAAACTGGTGAAGTCATATCATTGGA
TGGGACTAGTACACATTCCAACAAATGGATCTTGGCAGTGGGAAGATGGC
TCCATTCTCTCACCCAACCTACTAACAATAATTGAAATGCAGAAGGGAGA
CTGTGCACTCTATGCCTCGAGCTTTAAAGGCTATATAGAAAACTGTTCAA
CTCCAAATACATACATCTGCATGCAAAGGACTGTGTAAACGGCCGAGCGC
AACG

The final heavy- and light chain amino acid sequences of an exemplary construct comprising variable regions of murine anti-human CD3 antibody OKT3 with a human Fc sequence, and a human NKG2D sequence, can thus be based on the following sequences:

MDWVWTLLFLLSVTAGVHSQVQLQQSGAELARPGASVKMSCKASGYTFTR
YTMHWVKQRPGQGLEWIGYINPSRGYTNYNQKFKDKATLTTDKSSSTAYM
QLSSLTSEDSAVYYCARYYDDHYCLDYWGQGTTLTVSS
(SEQ ID NO:54-OKT3 heavy chain, amino acid 1-19
being a signal sequence)
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
(SEQ ID NO:55-human Fc part to be combined with
the above OKT3 heavy chain)
LFNQEVQIPLTESYCGPCPKNWICYKNNCYQFFDESKNWYESQASCMSQN
ASLLKVYSKEDQDLLKLVKSYHWMGLVHIPTNGSWQWEDGSILSPNLLTI
IEMQKGDCALYASSFKGYIENCSTPNTYICMQRTV
(SEQ ID NO:17-Ectodomain of human NKG2D to be
combined with Fc region)
MDFQVQIFSFLLISASVIISRGQIVLTQSPAIMSASPGEKVTMTCSASSS
VSYMNWYQQKSGTSPKRWIYDTSKLASGVPAHFRGSGSGTSYSLTISGME
AEDAATYYCQQWSSNPFTFGSGTKLEINRADTAPTVSIFPPSSEQLTSGG
ASVVCFLNNFYPKDINVKWKIDGSERQNGVLNSWTDQDSKDSTYSMSSTL
TLTKDEYERHNSYTCEATHKTSTSPIVKSFNRNEC
(SEQ ID NO:56-OKT3 light chain to be expressed
with the fusion protein OKT3heavychain-hFc-hNKG2D)

Functional mutations in the OKT3 sequences (e.g., VH) described in literature (e.g., Kipriyanov S M et al., Protein Eng. 1997; 10:445-53) can also be included.

Using similar methods, a variety of fusion proteins having features according to the inventive principles set forth herein, using, e.g., other antibody portions, other target-binding portions, or optional linkers, may be similarly designed and constructed.

### Example 4

The two constructs described in Example 1, respectively encoding the heavy chain of anti-mouse CD3-murine Fc-ectodomain of murine NKG2D and the light chain of anti mouse CD3, were co-expressed in mammalian cells, resulting in expression of the dimer protein. This protein was purified on a Protein A column (Pharmacia) to >90% purity (analyzed on SDS-PAGE and Western blot).

In order to test the functionality of the protein, FACS analyses were performed to investigate if the protein could bind the expected ligands. The N-terminal part of the protein (anti-mCD3) was expected to bind CD3 found on T cells, and the C-terminal part (mNKG2D) was expected to bind NKG2D ligands including Rea-1. Murine T cells were purified from fresh mouse spleen and the binding tested using FACS analyses which confirmed the binding. Furthermore, murine cancer cells were tested for NKG2D ligands, and the cell lines KLN205 and HEPA1-6 were chosen for further studies. Setting up an assay using real time electrical cell-substrate impedance sensing (ECIS) (Applied BioPhysics, Troy, N.Y.) ((Solly K et al (2004) ASSAY Drug Dev Technol 2, 363-372; Lo, C. M., Keese, C. R., and Giaever, I. (1995) Biophys. J. 69, 2800-2807 and Giaever, I., and Keese, C. R. (1993) Nature 366, 591-592) we showed an effect of the molecule in vitro. The cells (5×10³ for KLN205 and 5×10⁴ for HEPA1-6) were suspended in medium and seeded on electrodes. The cells were equilibrated in the incubator and the rate of cell proliferation on the microelectrodes was monitored as real-time changes in resistance. The following day murine T cells freshly purified from mouse spleen and incubated with IL-2 (800 U/ml) were added to the exponential growing cells in various ratios and the killing of the cancer cells followed for at least 24 hours.

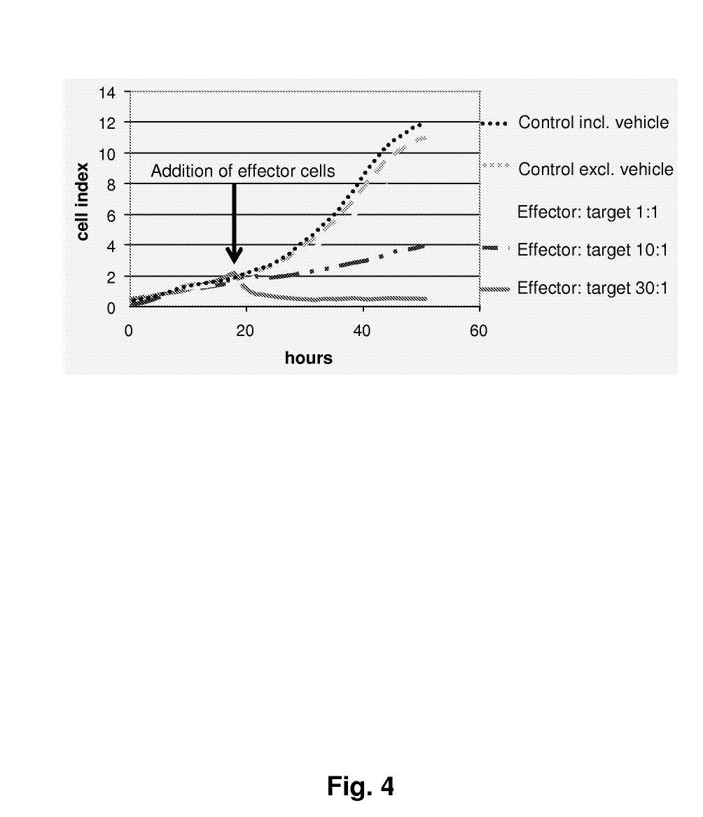

As seen in FIG. 4, the growth of KLN205 cells was affected by addition of effector cells. The more effector cells, the slower growth, and at an E:T ratio of 30:1 the KLN205 cells were completely killed.

At the E:T 10:1 ratio growth and killing was almost constant but did result in some additional growth of the KLN205 cells to a cell index of 4. We chose the E:T 10:1 ratio for testing the anti-mCD3-mFc-mNKG2D fusion protein.

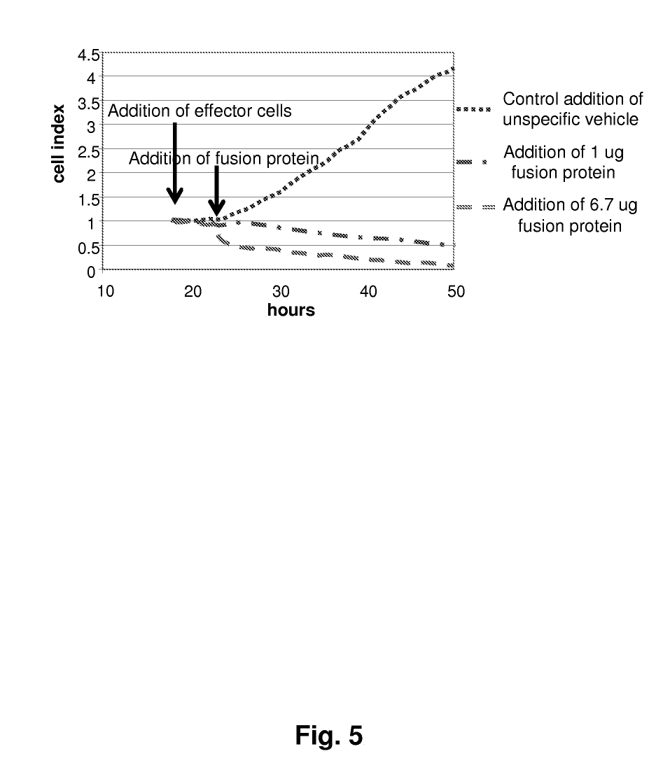

Again, KLN205 cells were equilibrated in the incubator and freshly purified murine T cells were added the following day. After 4 hours, either vehicle with an unspecific protein, or two different concentrations of the fusion protein, were added to the cells and the effect observed. As seen in FIG. 5, the killing of the KLN 205 cells was affected by the fusion protein in a dose dependent manner.

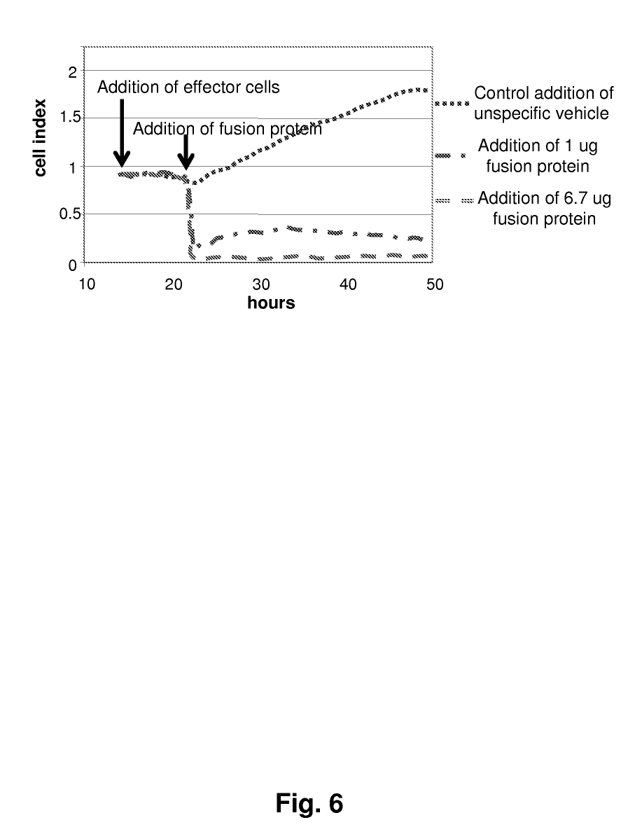

The same experiments were conducted with HEPA1-6 murine cells, and, at E:T 1:1, the growth and killing of HEPA1-6 resembled the pattern seen for KLN205. HEPA1-6 was also tested further and, as seen in FIG. 6, the killing of the HEPA1-6 cells was also affected by the fusion protein in a dose dependent manner.

### Aspects of the Invention

The following clauses describe specific aspects of the invention.

1. A multispecific protein comprising a first portion that corresponds to an antigen-binding portion of an effector lymphocyte activating receptor-specific antibody or a functional variant thereof, and a second portion that corresponds to a portion of a target-binding cell membrane protein or a functional variant thereof, wherein the second portion binds a cell-associated target that is different from the effector lymphocyte activating receptor, and the first portion does not bind the second portion.

2. The protein of clause 1, wherein the second portion binds a target that is expressed on cells that are regulated by effector lymphocytes in healthy subjects.

3. The protein of any of clauses 1 and 2, wherein the second portion comprises the target-binding portion of a type II membrane receptor or a functional variant thereof.

4. The protein of any of clauses 1-3, wherein the target-binding cell-membrane protein is a disulfide-linked C-type lectin.

5. The protein of any of clauses 1-4, wherein the target-binding cell-membrane protein is a natural killer (NK) cell receptor.

6. The protein of clause 5, wherein the NK cell receptor is selected from NKG2D, NKG2A/CD94, NKRP1, NKG2C/CD94, NKG2E/CD94, NKG2F/CD94, CD69, LLT1, AICL, and CD26.

7. The protein of clause 6, wherein the NK cell receptor is NKG2D.

8. The protein of any one of clauses 1-7, wherein the first portion corresponds to at least a portion of a monoclonal antibody against an activating receptor expressed on NK cells, T cells, NKT cells, or any combination thereof.

9. The protein of clause 8, wherein the activating receptor is expressed on NK cells.

10. The protein of clause 9, wherein the activating receptor is not NKG2D.

11. The protein of clause 8, wherein the activating receptor is CD3, CD4, CD8, CD16, CD28, CD16, NKp30, NKp44, or NKp46.

12. The protein of clause 11, wherein the activating receptor is CD3.

13. The protein of any one of clauses 1-12, wherein the first portion is indirectly bound to the second portion, the first and second portions being separated by a linker.

14. A multispecific protein comprising a first portion that corresponds to at least an antigen-binding portion of an effector lymphocyte activating receptor-specific antibody or a functional variant thereof, and a second portion that corresponds to a ligand-binding portion of human NKG2D or a functional variant thereof, wherein the effector lymphocyte activating receptor is not NKG2D.

15. The multispecific protein of clause 14, wherein the effector-lymphocyte activating receptor is activating receptor expressed on NK cells, T cells, NKT cells, or any combination thereof.

16. The multispecific protein of any of clauses 14 and 15, wherein the effector-lymphocyte activating receptor is CD3, CD4, CD8, CD16, CD28, CD16, NKp30, NKp44, or NKp46.

17. The multispecific protein of any of clauses 14-16, comprising the amino acid sequences of SEQ ID NO:17.

18. A pharmaceutically acceptable composition comprising a therapeutically effective amount of a protein according to any one of clauses 1-17 and at least one pharmaceutically acceptable carrier.

19. The composition of clause 18, further comprising at least one second therapeutic agent.

20. Use of the protein of any one of clauses 1-17, the composition of clause 18, or the composition of clause 19 in the preparation of a medicament to treat cancer.

21. A method of treating cancer in a mammal comprising delivering a therapeutically effective amount of a multispecific protein comprising a first portion that corresponds to an antigen-binding portion of an effector lymphocyte activating receptor-specific antibody or a functional variant thereof, and a second portion that corresponds to a portion of a target-binding cell membrane protein or a functional variant thereof, wherein the second portion binds a cell-associated target that is different from the effector lymphocyte activating receptor and is associated with a disease that is regulated by effector lymphocytes in healthy subjects.

22. The method of clause 21, wherein the target-binding cell membrane protein is NKG2D.

23. The method of any of clauses 21 and 22, wherein the protein is delivered to the mammal by administration of a pharmaceutically acceptable composition comprising a therapeutically effective dose of the protein and at least one pharmaceutically acceptable carrier.

24. The method of any of clauses 21-23, wherein the protein is delivered to the animal with one or more secondary anti-cancer agents.

25. The method of clause 24, wherein the protein and a second anti-cancer agent are delivered to the host as a single dosage form.

26. The method of any of clauses 21 and 22, wherein the protein is delivered to the mammal by administration of a nucleic acid encoding the protein to the mammal.

27. The method of any of clauses 21-26, wherein the mammal is a human diagnosed as suffering from a cancer.

28. Use of a multispecific protein comprising a first portion that corresponds to an antigen-binding portion of an effector lymphocyte activating receptor-specific antibody or a functional variant thereof, and a second portion that corresponds to a portion of a target-binding cell membrane protein or a functional variant thereof, in the preparation of a medicament for treating cancer in a mammal,

wherein the second portion binds a cell-associated target that is different from the effector lymphocyte activating receptor and is associated with a disease that is regulated by effector lymphocytes in healthy subjects.

29. The use of clause 28, wherein the mammal is a human diagnosed as suffering from a cancer.

30. A method for producing a multispecific protein comprising a first portion that corresponds to an antigen-binding portion of an effector lymphocyte activating receptor-specific antibody or a functional variant thereof, and a second portion that corresponds to a portion of a target-binding cell membrane protein or a functional variant thereof, wherein the second portion binds a cell-associated target that is different from the effector lymphocyte activating receptor, and the first portion does not bind the second portion, comprising providing one or more nucleic acids comprising sequences that encodes the first portion and second portion, such that expression of the fused nucleic acid leads to production of the multispecific protein, transfecting a cell that is able to express the fused nucleic acid with the fused nucleic acid, and maintaining the cell under conditions suitable for expression of the protein.

31. The method of clause 30, wherein the cell is contained in a non-human vertebrate host.

All references, including publications, patent applications, and patents, cited herein are hereby incorporated by reference to the same extent as if each reference were individually and specifically indicated to be incorporated by reference and were set forth in its entirety herein.

All headings and sub-headings are used herein for convenience only and should not be construed as limiting the invention in any way.

The use of the terms “a” and “an” and “the” and similar referents in the context of describing the invention are to be construed to cover both the singular and the plural, unless otherwise indicated herein or clearly contradicted by context.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

1. A multispecific protein comprising a first portion that corresponds to an antigen-binding portion of an effector lymphocyte activating receptor-specific antibody or a functional variant thereof, and a second portion that corresponds to a portion of a target-binding cell membrane protein or a functional variant thereof, wherein the second portion binds a cell-associated target that is different from the effector lymphocyte activating receptor, and the first portion does not bind the second portion.

2. The protein of claim 1, wherein the second portion binds a target that is expressed on cells that are regulated by effector lymphocytes in healthy subjects.

3. The protein of claim 1, wherein the second portion comprises the target-binding portion of a type II membrane receptor or a functional variant thereof.

4. The protein of claim 1, wherein the target-binding cell-membrane protein is a disulfide-linked C-type lectin.

5. The protein of claim 1, wherein the target-binding cell-membrane protein is a natural killer (NK) cell receptor.

6. The protein of claim 5, wherein the NK cell receptor is selected from NKG2D, NKG2A/CD94, NKRP1, NKG2C/CD94, NKG2E/CD94, NKG2F/CD94, CD69, LLT1, AICL, and CD26.

7. The protein of claim 6, wherein the NK cell receptor is NKG2D.

8. The protein of claim 1, wherein the first portion corresponds to at least a portion of a monoclonal antibody against an activating receptor expressed on NK cells, T cells, NKT cells, or any combination thereof.

9. The protein of claim 8, wherein the activating receptor is expressed on NK cells.

10. The protein of claim 9, wherein the activating receptor is not NKG2D.

11. The protein of claim 8, wherein the activating receptor is selected from CD3, CD4, CD8, CD16, CD28, CD16, NKp30, NKp44, and NKp46.

12. The protein of claim 11, wherein the activating receptor is CD3.

13. The protein of claim 1, wherein the first portion is indirectly bound to the second portion, the first and second portions being separated by a linker.

14. A multispecific protein comprising a first portion that corresponds to at least an antigen-binding portion of an effector lymphocyte activating receptor-specific antibody or a functional variant thereof, and a second portion that corresponds to a ligand-binding portion of human NKG2D or a functional variant thereof, wherein the effector lymphocyte activating receptor is not NKG2D.

15. The multispecific protein of claim 14, wherein the effector-lymphocyte activating receptor is activating receptor expressed on NK cells, T cells, NKT cells, or any combination thereof.

16. The multispecific protein of claim 14, wherein the effector-lymphocyte activating receptor is selected from CD3, CD4, CD8, CD16, CD28, CD16, NKp30, NKp44, and NKp46.

17. The multispecific protein of claim 14, comprising the amino acid sequences of SEQ ID NO:17.

18. A pharmaceutically acceptable composition comprising a therapeutically effective amount of a protein according to claim 1 and at least one pharmaceutically acceptable carrier.

19. The composition of claim 18, further comprising at least one second therapeutic agent.

20. A pharmaceutically acceptable composition comprising a therapeutically effective amount of a protein according to claim 14 and at least one pharmaceutically acceptable carrier.

21. A method of treating cancer in a mammal comprising delivering a therapeutically effective amount of a multispecific protein comprising a first portion that corresponds to an antigen-binding portion of an effector lymphocyte activating receptor-specific antibody or a functional variant thereof, and a second portion that corresponds to a portion of a target-binding cell membrane protein or a functional variant thereof, wherein the second portion binds a cell-associated target that is different from the effector lymphocyte activating receptor and is associated with a disease that is regulated by effector lymphocytes in healthy subjects.

22. The method of claim 21, wherein the target-binding cell membrane protein is NKG2D.

23. The method of claim 21, wherein the protein is delivered to the mammal by administration of a pharmaceutically acceptable composition comprising a therapeutically effective dose of the protein and at least one pharmaceutically acceptable carrier.

24. The method of any of claim 21, wherein the protein is delivered to the animal with one or more secondary anti-cancer agents.

25. The method of claim 24, wherein the protein and a second anti-cancer agent are delivered to the host as a single dosage form.

26. The method of any of claim 21, wherein the protein is delivered to the mammal by administration of a nucleic acid encoding the protein to the mammal.

27. The method of claim 21, wherein the mammal is a human diagnosed as suffering from a cancer.

28-29. (canceled)

30. A method for producing a multispecific protein comprising a first portion that corresponds to an antigen-binding portion of an effector lymphocyte activating receptor-specific antibody or a functional variant thereof, and a second portion that corresponds to a portion of a target-binding cell membrane protein or a functional variant thereof, wherein the second portion binds a cell-associated target that is different from the effector lymphocyte activating receptor, and the first portion does not bind the second portion, comprising providing one or more nucleic acids comprising sequences that encodes the first portion and second portion, such that expression of the fused nucleic acid leads to production of the multispecific protein, transfecting a cell that is able to express the fused nucleic acid with the fused nucleic acid, and maintaining the cell under conditions suitable for expression of the protein.

31. The method of claim 30, wherein the cell is contained in a non-human vertebrate host.


SEQUENCE LISTING (56 sequences)

SEQ ID NO: 1 - protein, 49 aa, Mus musculus
ASGVPAHFRGSGSGTSYSLTISGMEAEDAATYYCQQWSSNPFTFGSGTK

SEQ ID NO: 2 - protein, 58 aa, Mus musculus
QVQVQQSGAELARPGASVKMSCKASGYTFTRYTMLGMHWVKQRPGQGLEWIGYINPSR

SEQ ID NO: 3 - protein, 91 aa, Mus musculus
MAQVQLQQSGPELVKPGTSVKMSCKASGYKFISYVIHWVKQKPGQGLEWIGYINPYNAVTKYNEKFKGKATLTSDKSSSTASMELISLTSE

SEQ ID NO: 4 - protein, 115 aa, Mus musculus
QIVLTQSPAIMSASPGEKVTMTCSASSSVSYIHWYQQKSGTSPKRWIYDISKLASGVPVRFSGSGSGTSYSLTISSVEAEDAATYYCQQWSTNPPTFGAGTKLVLKRADAAPTVS

SEQ ID NO: 5 - protein, 5 aa, Mus musculus
SFPMA

SEQ ID NO: 6 - protein, 17 aa, Mus musculus
TISTSGGRTYYRDSVKG

SEQ ID NO: 7 - protein, 10 aa, Mus musculus
FRQYSGGFDY

SEQ ID NO: 8 - protein, 13 aa, Mus musculus
TLSSGNIENNYVH

SEQ ID NO: 9 - protein, 7 aa, Mus musculus
DDDKRPD

SEQ ID NO: 10 - protein, 9 aa, Mus musculus
HSYVSSFNV

SEQ ID NO: 11 - protein, 45 aa, Mus musculus
MGLSQVQLQESGPGLVKLPGTLSLTCAVSGGSISSSNWWSWVRQP

SEQ ID NO: 12 - protein, 34 aa, Mus musculus
MEFGLSWVFLVAILEGVHCEVQLVESGGGLVQPG

SEQ ID NO: 13 - protein, 137 aa, Mus musculus
MDRLTSSFLLLIVPAYVLSQVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSA

SEQ ID NO: 14 - protein, 131 aa, Mus musculus
METDTILLWVLLLWVPGSTGDTVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIK

SEQ ID NO: 15 - protein, 118 aa, Mus musculus
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSA

SEQ ID NO: 16 - protein, 111 aa, Mus musculus
IVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLELKR

SEQ ID NO: 17 - protein, 135 aa, Homo sapiens
LFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 18 - protein, 108 aa, Homo sapiens
IWVSQPPEIRAQEGTTASLPCSFNASRGKAAIGSATWYQDKVAPGMELSNVTPGFRGRVASFSASQFIRGHKAGLLIQDIQSHDARIYVCRVEVLGLGVGTGNGTRLV

SEQ ID NO: 19 - protein, 95 aa, Homo sapiens
TLPKPFIWAEPHFMVPKEKQVTICCQGNYGAVEYQLHFEGSLFAVDRPKPPERINKVKFYIPDMNSRMAGQYSCIYRVGELWSEPSNLLDLVVTE

SEQ ID NO: 20 - protein, 171 aa, Homo sapiens
QSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTPWTPRDLVSSQTQTQSCVPPTAGARQAPESPSTIPVPSQPQNSTLRPGPAAPIA

SEQ ID NO: 21 - protein, 148 aa, Homo sapiens
KNSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 22 - DNA, 23 bp, Artificial
  Primer
atgagggcccctactgtgtatcc

SEQ ID NO: 23 - DNA, 26 bp, Artificial
  Primer
ggacctctggctctaacactcattcc

SEQ ID NO: 24 - DNA, 16 bp, Artificial
  Primer
gtaaaacgacggccag

SEQ ID NO: 25 - DNA, 17 bp, Artificial
  Primer
caggaaacagctatgac

SEQ ID NO: 26 - DNA, 823 bp, Cricetulus migratorius
agggcgaattgggccctctagatgcatgctcgagcggccgccagtgtgatggatatctgcagaattcgcccttatgagggcccctactgtgtatcctgtgctcttgtttctttggtttacaggtgccatatgtgacatccagatgacccagtctccatcatcactgcctgcctccctgggagacagagtcactatcaattgtcaggccagtcaggacattagcaattatttaaactggtaccagcagaaaccagggaaagctcctaagctcctgatctattatacaaataaattggcagatggagtcccatcaaggttcagtggcagtggttctgggagagattcttctttcactatcagcagcctggaatccgaagatattggatcttattactgtcaacagtattataactatccgtggacgttcggacctggcaccaagctggaaatcaaacgggctgatgctaagccaaccgtctccatcttcccaccatccagtgagcagttgggcactggaagtgccacacttgtgtgcttcgtgaacaacttctaccccaaagacatcaatgtcaagtggaaagtagatggcagtgaaaaacgagatggcgtcctgcagagtgtcactgatcaggacagcaaagacagcacctacagcctgagcagcaccctctcgctgaccaaagcagattatgagaggcataacctgtatacctgtgaggttactcataagacatcaactgcagccattgtcaagaccctgaacaggaatgagtgttagagcagaggtccaagggcgaattccagcacactggcggccgttact

SEQ ID NO: 27 - DNA, 22 bp, Artificial
  Primer
gtactccctctcaaaagcgggc

SEQ ID NO: 28 - DNA, 24 bp, Artificial
  Primer
ctgaagggattacatgcactgccc

SEQ ID NO: 29 - DNA, 932 bp, Cricetulus migratorius
tatacgactcactatagggcgaattgggccctctagatgcatgctcgagcggccgccagtgtgatggatatctgcagaattcgcccttcagccctggattcccaggtcctcccattctgtgatcagcactgaacacaggccacttgccatgaactcaggactccaattggttttctttgtcctcactctaaaaggtatacagggtgaggtgcagctggtggagtctgggggaggcttggtgcagcctggaaagtccctgaaactctcctgtgaggcctctggattcaccttcagcggctatggcatgcactgggtccgccaggctccagggagggggctggagtcggtcgcatacattactagtagtagtattaatatcaaatatgctgacgctgtgaaaggccggttcaccgtctccagagacaatgccaagaacttactgtttctacaaatgaacattctcaagtctgaggacacagccatgtactactgtgcaagattcgactgggacaaaaattactggggccaaggaaccatggtcaccgtcgcctcagccaaaacaacagccccaaagggcgaattccagcacactggcggccgttactagtggatccgagctcggtaccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctca

SEQ ID NO: 30 - DNA, 23 bp, Artificial
  Primer
cagccctggattcccaggtcctc

SEQ ID NO: 31 - DNA, 25 bp, Artificial
  Primer
tggggctgttgttttggctgaggag

SEQ ID NO: 32 - DNA, 724 bp, Mus musculus
ctggggatgctggtcgcttccgtgctagcagtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacaaaaccccgggaggagcagttcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaaggatccttattca

SEQ ID NO: 33 - DNA, 48 bp, Artificial
  Primer
taacatccttgaattcacttgccaccatgaactcaggactccaattgg

SEQ ID NO: 34 - DNA, 32 bp, Artificial
  Primer
gttgagaatcagctgtgcaacaaggaagtccc

SEQ ID NO: 35 - DNA, 38 bp, Artificial
  Primer
taaccatggcggccgtttttacaccgcccttttcatgc

SEQ ID NO: 36 - DNA, 955 bp, Mus musculus
gccttttgctcacatgttctttcctgcgtaatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcgcccttgttgagaatcagctgtgcaacaaggaagtcccagtttcctcaagagagggctactgtggcccatgccctaacaactggatatgtcacagaaacaactgttaccaattttttaatgaagagaaaacctggaaccagagccaagcttcctgtttgtctcaaaattccagccttctgaagatatacagtaaagaagaacaggatttcttaaagctggttaagtcctatcactggatgggactggtccagatcccagcaaatggctcctggcagtgggaagatggctcctctctctcatacaatcagttaactctggtggaaataccaaaaggatcctgtgctgtctatggctcaagctttaaggcttacacagaagactgtgcaaatctaaacacgtacatctgcatgaaaagggcggtgtaaaaacggccgccatggttaaagggcgaattctgcagatatccatcacactggcggccgctcgagcatgcatctagagggcccaattcgccctatagtgagtcgta

SEQ ID NO: 37 - protein, 601 aa, Artificial
  Fusion protein
MNSGLQLVFFVLTLKGIQGEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSATTTAPSVYPLAPACDSTTSTTNTVTLGCLVKGYFPEPVTVSWNSGALTSGVHTFPSVLHSGLYSLSSSVTVPSSTWPSQTVTCNVAHPASSTKVDKKIVPRGDPRDCGCKPCICTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKDLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 38 - protein, 234 aa, Cricetulus migratorius
MRAPTVYPVLLFLWFTGAICDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRADAKPTVSIFPPSSEQLGTGSATLVCFVNNFYPKDINVKWKVDGSEKRDGVLQSVTDQDSKDSTYSLSSTLSLTKADYERHNLYTCEVTHKTSTAAIVKTLNRNEC

SEQ ID NO: 39 - DNA, 30 bp, Artificial
  Primer
aagcagtggtatcaacgcagagtacgcggg

SEQ ID NO: 40 - DNA, 45 bp, Artificial
  Primer
ctaatacgactcactatagggcaagcagtggtatcaacgcagagt

SEQ ID NO: 41 - DNA, 22 bp, Artificial
  Primer
ctaatacgactcactatagggc

SEQ ID NO: 42 - DNA, 37 bp, Artificial
  Primer
ggcccggggggccttaacactcattcctgttgaagct

SEQ ID NO: 43 - DNA, 24 bp, Artificial
  Primer
ttatttaccaggagagtgggagag

SEQ ID NO: 44 - DNA, 22 bp, Artificial
  Primer
tcatttacccagagaccgggag

SEQ ID NO: 45 - DNA, 22 bp, Artificial
  Primer
tcatttacccggagaccgggag

SEQ ID NO: 46 - DNA, 1386 bp, Mus musculus
caatgaatgatacgccagcttggtaccgagctcggatccactagtaacggccgccagtgtgctggaattcgcccttctaatacgactcactatagggcaagcagtggtatcaacgcagagtacgcggggcttctcttgggtcttagacaaacgtatccattaaattcaaatattcttctggcagtcaagaaaagaagcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaattaaagggctagtcagagctccacaagtatacatcttgccgccaccagcagagcagttgtccaggaaagatgtcagtctcacttgcctggtcgtgggcttcaaccctggagacatcagtgtggagtggacagcatgggcatacggaggagactacagggaccggcacagtctggactctgacggtcttactcatttttgcaaggtcatatgaaacaagcaaggggaaaacagaattcttctcagccactgggaaccagggttaaaaatatcaccggagaaaaacccccccggccccgggaaagaaaggggaatttcccaaattttcccatctggggccctcaaataattaagggccccacccccccaaaggggaaaaaaataagggggcggttttataccggaggggaaaaagaggtgtgaaaataatatttgtggaaacccttccccgcgggggagagagaaagagcccgccacccctcccccccccccgcggggaggagggcgc

SEQ ID NO: 47 - DNA, 1010 bp, Mus musculus
gggcgaattgggccctctagatgcatgctcgagcggccgccagtgtgatggatatctgcagaattcgcccttaagcagtggtatcaacgcagagtacgcggggagagatggagacagacacactcctgttatgggtactgctgctctgggttccaggttccactggtgacattgtgctgacacggtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcacccccaacatccatcctgtggaggaggaggatgctgcaacctattactgtcagcacattagggagcttacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgtcgaccaaggacgagtatgaacgacataacagctatacctgtgaggccactcacaagacatcaacttcacccattgtcaagagcttcaacaggaatgagtgttaaggccccccgggccaagggcgaattccagcacactggcggccgttactagtggatccgagctcggtaccaagcttggcgtaatcatggtcatagctgtttcctgtgtgaaattgttatccgctcacaattccacacaacatacgagccggaagcataagtgtaagcctgggtgcctaatgagtgagctact

SEQ ID NO: 48 - DNA, 702 bp, Homo sapiens
ctcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 49 - DNA, 22 bp, Artificial
  Primer
agtatttgatggggtggattcg

SEQ ID NO: 50 - DNA, 24 bp, Artificial
  Primer
gatggttgatcatctttacacagt

SEQ ID NO: 51 - DNA, 57 bp, Artificial
  Primer
cgttgcgctgctagccgttgcgctggatccttattcaaccaagaagttcaaattccc

SEQ ID NO: 52 - DNA, 42 bp, Artificial
  Primer
cgttgcgctcggccgtttacacagtcctttgcatgcagatgt

SEQ ID NO: 53 - DNA, 454 bp, Homo sapiens
cgttgcgctgctagccgttgcgctggatccttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtgtaaacggccgagcgcaacg

SEQ ID NO: 54 - protein, 138 aa, Mus musculus
MDWVWTLLFLLSVTAGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 55 - protein, 196 aa, Homo sapiens [sequence omitted]

SEQ ID NO: 56 - protein, 235 aa, Mus musculus
MDFQVQIFSFLLISASVIISRGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC